(19) United States
(12) Patent Application Publication
(10) Pub. No.: US 2026/0366067 A1
(43) Pub. Date: Nov. 19, 2026

(54) THICK TISSUE TRANSCRIPTOMICS AND TRANSLATOMICS

(71) Applicants: The Broad Institute, Inc., Cambridge, MA (US); Massachusetts Institute of Technology, Cambridge, MA (US)

(72) Inventors: Xiao WANG, Cambridge, MA (US); Xin SUI, Cambridge, MA (US)

(73) Assignees: The Broad Institute, Inc., Cambridge, MA (US); Massachusetts Institute of Technology, Cambridge, MA (US)

(21) Appl. No.: 19/709,449

(22) Filed: Jun. 16, 2026

Related U.S. Application Data
(63) Continuation of application No. PCT/US2024/060469, filed on Dec. 17, 2024.
(60) Provisional application No. 63/611,403, filed on Dec. 18, 2023.
(60) Provisional application No. 63/659,690, filed on Jun. 13, 2024.

(51) Int. Cl. C12Q 1/6855; C12Q 1/25; C12Q 1/6809; C12Q 1/6813; C12Q 1/6869; C12Q 1/6886

(52) U.S. Cl. CPC C12Q 1/6855; C12Q 1/25; C12Q 1/6809; C12Q 1/6813; C12Q 1/6869; C12Q 1/6886; C12Q 2600/158

(57) ABSTRACT

The present disclosure provides methods (referred to herein as “Thick-STARmap” and “Thick-RIBOmap”), probes, compositions, kits, and systems for profiling RNA expression and/or translation in thick tissue (e.g., 20-350 micron) samples. Also provided by the present disclosure are methods for diagnosing a disease or disorder in a subject based on a profile of RNA expression and/or translation in a cell tissue, or other biological sample. Methods of screening for or testing a candidate agent capable of modulating RNA expression and/or translation are also provided by the present disclosure. The present disclosure also provides methods for treating a disease or disorder in a subject in need thereof. Oligonucleotide probes useful for performing the methods described herein are also provided by the present disclosure. Additionally, the present disclosure provides kits comprising any combination of the oligonucleotides described herein.

### RELATED APPLICATIONS

This application is a continuation of International Application No. PCT/US2024/060469, filed on Dec. 17, 2024, and claims priority under 35 U.S.C. § 119 (e) to U.S. Provisional Application, U.S. 63/611,403, filed Dec. 18, 2023, and to U.S. Provisional Application, U.S. 63/659,690, filed Jun. 13, 2024, each of which is incorporated herein by reference.

### REFERENCE TO AN ELECTRONIC SEQUENCE LISTING

The contents of the electronic sequence listing (114203-3044_SL.xml; Size: 36,299 bytes; and Date of Creation: Jun. 9, 2026) are herein incorporated by reference in its entirety.

### BACKGROUND

In biological tissues, functional heterogeneity originates partly from the heterogeneity of cell-specific gene and protein expression patterns, which determine the characteristic three-dimensional (3D) molecular architecture and cellular features inherent to distinct tissue types. Single-cell RNA sequencing (scRNA-seq) enables the mapping of the transcriptome, providing insights into the repertoire of mRNAs that reflect gene transcription and regulatory mechanisms. Ribosome profiling offers the ability to map the translatome, enabling the analysis of protein translation at a transcriptome-wide scale and providing information on protein synthesis and post-transcriptional regulation. Furthermore, in situ sequencing technologies have bridged the gap in investigating the spatial organization of molecular underpinnings and examining the transcriptome¹ and translatome² within their native spatial context. While most techniques are confined to analyzing thin tissue sections (10-20 μm), characterizing the transcriptome and translatome at cellular resolution in thicker tissues (20-350 μm) could significantly expand the visualization of three-dimensional associations among cell types in tissue architectures. Moreover, an axial depth of 200 μm corresponds to the tissue scales utilized in various neuroscience data-gathering approaches, including electrophysiology, activity-based imaging, and three-dimensional morphological studies. Such an integration of data streams could provide unprecedented insight into cellular function and interactions within the native 3D tissue environment, enabling accurate study and assessment of biological processes at the cellular level. Accordingly, techniques for profiling transcriptomics and/or translatomics in thick tissue samples are needed.

### SUMMARY

The present disclosure describes the development of methods and systems for in situ sequencing of RNAs of interest in thick tissue samples referred to herein as “thick-STARmap” and “thick-RIBOmap.” RNA profiling strategies have been described in, for example, International PCT Application Publication WO 2019/199579, published Oct. 17, 2019, International PCT Application Publication WO 2022/236011, published Nov. 10, 2022, International PCT Application Publication WO 2022/251586, published Dec. 1, 2022, International PCT Application Publication WO 2022/178274, published Aug. 25, 2022, International PCT Application Publication WO 2023/018756, published Feb. 16, 2023, and International PCT Application Publication WO 2023/278409, published Jan. 5, 2023, each of which is hereby incorporated by reference. In comparison to previously disclosed methods, the methods described herein enhance diffusional access of enzymes (e.g., ligases, polymerases) in large-tissue volumes by utilizing a modified approach involving embedding probes in a hydrogel and digesting the tissue before carrying out enzymatic reactions. The improved methods enable a sequencing-based approach for high-content three-dimensional in situ quantification of hundreds to thousands of gene transcripts and their respective translation within an intact thick tissue sample. Thick-STARmap and thick-RIBOmap are capable of enhancing understanding of the complex interplay between transcriptional and translational regulation within complex three-dimensional tissue microenvironments at cellular resolution levels.

Thus, in various aspects, the present disclosure provides methods, uses, compositions, kits, and systems for profiling RNA expression and/or translation in thick tissue samples, e.g., tissue samples that are more than 10-20 μm thick, or at least 20, at least 30, at least 40, at least 50, at least 100, at least 150, at least 200, at least 250, at least 300, or at least 350 μm thick. In some embodiments, the tissue samples comprise a fixed tissue sample or other biological sample (e.g., a population of cells in a tissue or an organ of the body).

In the methods disclosed herein, a tissue sample may be contacted with one or more sets of oligonucleotide probes under conditions that allow for specific hybridization of the probes to RNAs in the intact tissue. One or more of the probes comprises at least one crosslinking moiety (for example, an acrydite moiety, e.g., at the 5′ end of the probe), allowing the probes and tissue sample to be embedded in a hydrogel matrix prior to any subsequent steps in the method. Following tissue digestion and the removal of unbound biomolecules (e.g., lipids and proteins), cDNA amplicons with unique gene identifiers are synthesized via enzymatic ligation and rolling circle amplification (RCA). The cDNA amplicons are then embedded in a polymeric matrix and sequenced to determine the identity of the transcripts and their location within the polymetric matrix (e.g., through SEDAL sequencing (Sequencing with Error-reduction by Dynamic Annealing and Ligation) as described further herein). In certain embodiments, the methods, compositions, and systems disclosed herein may allow for the profiling of ribosome-bound RNA to examine RNA translation across the transcriptome.

Based on the locations of the transcripts, spatiotemporal information may be obtained to improve the understanding of how RNA expression and/or translation affects cellular function in health and disease. The methods, compositions, and systems may be useful for comparing RNA expression and/or translation in, for example, diseased and healthy tissue samples; or for comparing RNA expression and/or translation in, for example, a tissue sample treated with an agent (e.g., a therapeutic agent or potential therapeutic agent, such as a small molecule, a protein, a peptide, a nucleic acid, a lipid, or a carbohydrate, or a research agent) and an untreated tissue sample (e.g., developing tissues, normal tissues, diseased tissues, or aging tissues).

In one aspect, the present disclosure provides a method for profiling RNA expression and/or translation in a tissue sample comprising the steps of:
a) contacting the tissue sample with 1) one or more sets of oligonucleotide probes, wherein each set of oligonucleotide probes comprises a first probe and a second probe, and 2) a third oligonucleotide probe, wherein
   i) the first probe comprises a portion that is complementary to at least a portion of the third probe, a portion that is complementary to an RNA of interest, and a portion that is complementary to a portion of the second probe;
   ii) the second probe comprises a portion that is complementary to a portion of the first probe, a portion that is complementary to the RNA of interest, and an oligonucleotide barcode sequence; and
   iii) the third probe comprises a portion that is complementary to the first probe and at least one crosslinking moiety;
b) co-polymerizing the one or more sets of probes, the third probe, and a polymeric matrix to form an oligonucleotide-polymeric matrix hybrid, wherein the first probe is hybridized to the RNA of interest;
c) removing unbound biomolecules from the oligonucleotide-polymeric matrix hybrid;
d) ligating the 5′ end and the 3′ end of the second probe together to produce a circular oligonucleotide;
e) performing rolling circle amplification to amplify the circular oligonucleotide using the first probe as a primer, thereby producing one or more concatenated amplicons;
f) embedding the one or more concatenated amplicons in the polymeric matrix; and
g) sequencing the concatenated amplicons, or a portion thereof, embedded in the polymeric matrix to determine the identity and location of each RNA of interest in the tissue sample.

In some embodiments, the method may be used for profiling RNA translation in a tissue sample. In some embodiments, the set of probes comprises a fourth probe comprising a portion complementary to the third probe, a portion complementary to at least a portion of a ribosomal RNA (rRNA), and a portion complementary to the second probe. In some embodiments, the portion of the fourth probe complementary to at least a portion of an rRNA is complementary to at least a portion of an 18S rRNA.

These methods can thus be used to determine the expression patterns, translation of RNAs, and locations of RNAs within tissues (e.g., developing tissues, normal tissues, diseased tissues, treated tissues).

In some embodiments, the tissue comprises cells of multiple different cell types. In some embodiments, the tissue is a fixed tissue sample. In some embodiments, the RNAs comprise known sequences of interest. In some embodiments, more than 1, more than 2, more than 3, more than 4, more than 5, more than 10, more than 20, more than 30, more than 40, more than 50, more than 100, more than 200, more than 500, more than 1000, more than 2000, more than 3000, more than 4000, more than 5000, more than 6000, more than 7000, more than 8000, more than 9000, or more than 10,000 RNAs of interest are profiled simultaneously using the methods described herein. In some embodiments, the method further comprises profiling additional molecules within the tissue sample.

In another aspect, the present disclosure provides methods for profiling RNA expression and/or translation in a tissue sample comprising the steps of:
a) contacting the tissue sample with 1) one or more sets of oligonucleotide probes, wherein each set of oligonucleotide probes comprises a first probe and a second probe, and 2) a third oligonucleotide probe, wherein
   i) the first probe comprises a portion that is complementary to at least a portion of the third probe, a portion that is complementary to an RNA of interest, and a portion that is complementary to a portion of the second probe;
   ii) the second probe comprises a portion that is complementary to a portion of the first probe, a portion that is complementary to the RNA of interest, and an oligonucleotide barcode sequence; and
   iii) the third probe comprises a portion that is complementary to the first probe and at least one crosslinking moiety;
b) co-polymerizing the one or more sets of probes, the third probe, and a polymeric matrix to form an oligonucleotide-polymeric matrix hybrid, wherein the first probe is hybridized to the RNA of interest;
c) ligating the 5′ end and the 3′ end of the second probe together to produce a circular oligonucleotide;
d) performing rolling circle amplification to amplify the circular oligonucleotide using the first probe as a primer, thereby producing one or more concatenated amplicons;
e) embedding the one or more concatenated amplicons in the polymeric matrix; and
f) sequencing the concatenated amplicons, or a portion thereof, embedded in the polymeric matrix to determine the identity and location of each RNA of interest in the tissue sample.

The methods, uses, probes, compositions, kits, and systems described herein may be useful for studying RNA expression and translation patterns in tissues for diagnosing, preventing, and treating various diseases and disorders, for affecting development, for research purposes, for drug discovery, and for any other purposes recognized by one of skill in the art. Thus, in one aspect, the present disclosure provides methods for diagnosing a disease or disorder in a subject. For example, the methods for profiling RNA expression and/or translation described herein may be performed on a tissue sample, or on multiple tissue samples, taken from a subject (e.g., a subject who is thought to have or is at risk of having a disease or disorder, or a subject who is healthy or thought to be healthy). The expression and/or translation of various RNAs in the tissue(s) can then be compared to the expression and/or translation of the same or other RNAs in one or more non-diseased tissue samples (e.g., tissue from a healthy individual, or multiple tissues from a population of healthy individuals). Any difference in the RNA expression and/or translation profiles of the tissue (including of a single RNA or of multiple RNAs, e.g., a specific disease signature) relative to one or more non-diseased tissues may indicate that the subject has the disease or disorder. RNA expression and/or translation in one or more non-diseased tissues (e.g., healthy tissues) may be profiled alongside expression and/or translation in a diseased tissue as a control experiment. RNA expression and/or translation in one or more non-diseased tissues (e.g., healthy cells) may have also been profiled previously, and the profile of a diseased tissue may be compared to reference data for a non-diseased tissue (e.g., healthy tissue).

In another aspect, the present disclosure provides methods of screening for an agent (e.g., a therapeutic agent, a research agent, or any kind of stimulus, such as a mechanical force, light, heat, electricity, etc.) capable of modulating RNA expression and/or translation. For example, the methods for profiling RNA expression and/or translation described herein may be performed in a tissue sample in the presence of one or more candidate agents. The expression and/or translation of various RNAs in the tissue (e.g., a healthy tissue, or a diseased tissue) can then be compared to the expression and/or translation of the RNAs in a tissue that was not exposed to the one or more candidate agents. Any difference in the RNA expression and/or translation profiles relative to the tissue that was not exposed to the candidate agent(s) may indicate that expression and/or translation of particular RNAs is modulated by the candidate agent(s). In some embodiments, a particular signature (e.g., of altered expression and/or translation of multiple RNAs) that is known to be associated with the treatment of a disease may be used to identify agents capable of modulating RNA expression and/or translation in a desired manner and thus treating a disease. The methods, compositions, and systems described herein may also be used to identify drugs that have certain side effects, for example, by looking for particular RNA expression and/or translation patterns associated with a side effect when one or more tissues are treated with a candidate agent or known drug (or combinations of multiple candidate agents and/or known drugs, e.g., as provided in a screening library of compounds). The methods and systems described herein may also be used to identify research reagents or chemical probes that may be useful for studying RNA expression, RNA translation, RNA location, RNA processing, RNA mutations (e.g., single base substitutions), etc.

In yet another aspect, the present disclosure provides methods for treating a disease or disorder in a subject. For example, the methods and systems for profiling RNA expression and/or translation described herein may be performed on a tissue sample taken from a subject (e.g., a subject who is thought to have or is at risk of having a disease or disorder, or a subject diagnosed with a disease or disorder). The RNA expression and/or translation profiles can then be compared to the RNA expression and/or translation profiles of a non-diseased tissue sample. A treatment for the disease or disorder (e.g., a pharmaceutical agent, surgery, radiation therapy, surgery, physical therapy, lifestyle changes, exercise, diet, etc.) may then be administered to or prescribed for the subject if any difference is observed in the RNA expression and/or translation profiles relative to reference data for a non-diseased tissue. RNA expression and/or translation in one or more non-diseased tissues may be profiled alongside RNA expression and/or translation in a diseased tissue as a control experiment. RNA expression and/or translation in one or more non-diseased tissues (e.g., healthy tissues) may have also been profiled previously, and RNA expression and/or translation in a diseased tissue may then be compared to reference data for a non-diseased tissue.

In another aspect, the present disclosure provides sets of oligonucleotide probes comprising; i) a first probe (also referred to herein as the “primer” probe) comprising a portion that is complementary to at least a portion of the third probe (also referred to herein as the “adapter” probe), a portion that is complementary to an RNA of interest, and a portion that is complementary to a portion of the second probe (also referred to herein as the “padlock” probe); ii) a second probe comprising a portion that is complementary to a portion of the first probe, a portion that is complementary to the RNA of interest, and an oligonucleotide barcode sequence; and iii) a third probe comprising a portion that is complementary to the first probe and at least one crosslinking moiety. In some embodiments, a set of probes comprises a fourth probe (also referred to herein as the “splint” probe) comprising a portion complementary to the third probe, a portion complementary to at least a portion of a ribosomal RNA (rRNA) within a ribosome, and a portion complementary to the second probe.

In a further aspect, the present disclosure provides pluralities of probes comprising multiple sets of probes, wherein each set of probes comprises a first probe that is complementary to a different RNA. In certain embodiments, the plurality comprises more than 1, more than 10, more than 100, more than 1000, or more than 10,000 sets of probes.

In another aspect, the present disclosure provides kits (e.g., a kit comprising any of the probes, sets of probes, or pluralities of probes disclosed herein). In some embodiments, the kits described herein may also include any other reagents or components useful in performing the methods described herein, including, but not limited to, enzymes (such as a ligase, a polymerase (e.g., a DNA polymerase), and/or a protease), nucleotides comprising a reactive moiety (e.g., a nucleophile, such as in amine-modified nucleotides), buffers, reagents (including dyes, stains, and more), and monomers for making a polymeric matrix (e.g., a polyacrylamide matrix).

In a further aspect, the present disclosure provides samples comprising one or more concatenated amplicons or polymeric matrix-embedded concatenated amplicons produced by any of the methods described herein.

In another aspect, the present disclosure provides a system for profiling RNA expression and/or translation in a cell comprising:
a) a tissue sample; and
b) one or more sets of oligonucleotide probes, wherein each set of probes comprises a first probe and a second probe, and a third oligonucleotide probe, wherein:
   i) the first probe comprises a portion that is complementary to at least a portion of the third probe, a portion that is complementary to an RNA of interest, and a portion that is complementary to a portion of the second probe;
   ii) the second probe comprises a portion that is complementary to a portion of the first probe, a portion that is complementary to the RNA of interest, and an oligonucleotide barcode sequence; and
   iii) the third probe comprises a portion that is complementary to the first probe and at least one crosslinking moiety.

In some embodiments, the systems may be used for profiling RNA translation in a cell. In some embodiments, the system further comprises a fourth probe comprising a portion complementary to the third probe, a portion complementary to at least a portion of a ribosomal RNA (rRNA) within a ribosome, and a portion complementary to the second probe.

Any of the oligonucleotide probes (i.e., the sets and pluralities probes) described herein may be used in the systems contemplated by the present disclosure. In some embodiments, the system further comprises protease. In some embodiments, the system further comprises a DNA ligase. In some embodiments, the system further comprises a DNA polymerase. In some embodiments, the system further comprises nucleotides comprising a reactive moiety (e.g., a nucleophile, such as in amine-modified nucleotides). In some embodiments, the system further comprises buffers, reagents (including dyes, stains, and more) and monomers for preparing a polymeric matrix. In some embodiments, the system further comprises a microscope. In some embodiments, the system further comprises a computer. In some embodiments, the system further comprises a camera.

The foregoing concepts, and additional concepts discussed below, may be arranged in any suitable combination, as the present disclosure is not limited in this respect. Further, other advantages and novel features of the present disclosure will become apparent from the following detailed description of various non-limiting embodiments when considered in conjunction with the accompanying figures.

### BRIEF DESCRIPTION OF THE DRAWINGS

The following drawings form part of the present specification and are included to further demonstrate certain aspects of the present disclosure, which can be better understood by reference to one or more of these drawings in combination with the Detailed Description of Certain Embodiments presented herein.

FIGS. 1A-1C show thick-STARmap workflow and probe integration strategies. FIG. 1A provides a schematic summary of thick-STARmap: in situ sequencing of transcriptional states in thick tissue blocks. Following thick tissue slice preparation, primer (first probe) and padlock (second probe) probes with unique gene identifiers hybridize to intracellular mRNAs (dashed line) in the intact tissue. The primer probe (first probe) features a flanking linker sequence at its 5′ end and is covalently crosslinked (rhombus) to an acrydite-modified oligonucleotide adapter probe (third probe). The probe set is copolymerized with acrylamide, forming a DNA-gel hybrid (wavy lines) through the adapter probe's functionalized acrylic group, followed by the removal of unbound lipids and proteins. Subsequently, enzymatic ligation and RCA are used to construct in situ cDNA amplicons, and barcodes on the unique gene identifiers are read out via cyclic in situ sequencing with error reduction by dynamic annealing and ligation (SEDAL). The comprehensive quantification of RNA enables the elucidation of gene expression patterns and the identification of cell types within the native 3D tissue context. FIG. 1B shows a comparison of two strategies for integrating probes into a hydrogel matrix: the top panel demonstrates the scalable method of incorporating acrydite modification into the adapter probe (third probe), while the bottom panel displays the non-scalable technique involving acrydite modification in the primer (first probe). FIG. 1C shows the mechanism of the photo-crosslinking reaction between ^{CNV}K and pyrimidines via a [2+2] cycloaddition reaction upon UV-A irradiation (366 nm).

FIGS. 2A-2C show optimization of thick-STARmap. FIGS. 2A-2B show evaluation of probe anchoring efficiency in a hydrogel matrix using different strategies. Raw fluorescence images illustrating the effects of (1) adapter probe presence, (2) adapter probe hybridization or UV crosslinking, and (3) the use of VA044 thermal initiator. Full field: 465 μm by 465 μm, scale bar, 100 μm. All fluorescent images show Ch1 to Ch4 (color-coded channels for barcode decoding) and cell nuclei in mouse brain slices. FIG. 2C shows 15% TBE-Urea gels demonstrating UV crosslinking efficiency with varying adapter-primer (third-first probe) ratios. The ^{CNV}K- and acrydite-containing adapter probe (third probe) used for UV crosslinking is [5Acryd]GCTA [cnvK]ATACGTCGTACTGGTAGG [Inv-dT] (24 nt) (SEQ ID NO: 1). Primer probe used is 58 bp ssDNA with a 24 bp flanking liner at the 5′ end. M, Marker: IDT ssDNA 20/100 Ladder.

FIGS. 3A-3C show homogeneous distribution of cDNA amplicons in 200 μm 3D tissue using thick-STARmap. FIGS. 3A-3B show a 3D visualization of amplicon spatial organization. Full field: 465 μm by 465 μm by 200 μm. FIG. 3C provides raw fluorescence images at various depths in the thick-STARmap sample. Full field: 465 μm by 465 μm. Scale bar, 100 μm. All fluorescent images show Ch1 to Ch4 (color-coded channels for barcode decoding) and cell nuclei in mouse brain slices.

FIGS. 4A-4F show spatially resolved RNA expression profiling in thick tissue. FIGS. 4A-4C provide 3D fluorescent images of in-process thick-STARmap. FIGS. 4A-4B provide 3D-reconstructed fluorescence images of the tissue-hydrogel network of in situ RNA sequencing for 60 genes. FIG. 4C provides 3D-reconstructed fluorescence image from four rounds of cyclic imaging. Full field: 3.0 mm by 3.0 mm by 100 μm. All fluorescent images show Ch1 to Ch4 (color-coded channels for barcode decoding) and cell nuclei in mouse brain slices. FIGS. 4D-4E show three-dimensional spatial distribution of major cell types.

FIG. 4F shows a uniform manifold approximation plot (UMAP) visualization representing major cell types across all sequenced cells clustered by Leiden clustering. FIGS. 4D-4F use the same color code for cell types.

FIGS. 5A-5C show design and validation of thick-RIBOmap. FIG. 5A provides a schematic summary of thick-RIBOmap: in situ sequencing of translatome states in thick tissue blocks. Following thick tissue slice preparation, primer (first probe) and padlock (second probe) probes with unique gene identifiers hybridize to intracellular mRNAs (dashed line), while splint probes (fourth probes) bind to 18S rRNA of ribosomes in the intact tissue. Splint probes serve as templates for proximity ligation and circularizing padlock probes (second probes). Both primer probe (first probe) and splint probe (fourth probe) feature a flanking linker sequence at their 5′ ends and are covalently crosslinked (rhombus) to an acrydite-modified oligonucleotide adapter probe (third probe). The probe set is copolymerized with acrylamide, forming a DNA-gel hybrid (wavy lines) through the adapter's functionalized acrylic group, followed by the removal of unbound lipids and proteins. Subsequently, enzymatic ligation and RCA are used to construct in situ cDNA amplicons, and barcodes on the unique gene identifiers are read out via cyclic in situ sequencing with error reduction by dynamic annealing and ligation (SEDAL). The comprehensive quantification of ribosome-bound RNA enables the deciphering of gene translation patterns and the detection of protein synthesis across the entire transcriptome. FIGS. 5B-5C show 3D visualization of amplicon spatial organization. FIG. 5B is a raw fluorescence image merging four distinct fluorescence channels. FIG. 5C shows four raw fluorescence images displaying four separate fluorescence channels, with each color representing a specific gene as indicated in the legend of FIG. 5B. Full field: 465 μm by 465 μm by 200 μm.

FIGS. 6A-6F show that thick-STARmap and thick-RIBOmap enable spatiotemporally resolved transcriptomics and translatomics in 200 μm thick tissue blocks. FIG. 6A provides a schematic summary of thick-STARmap and thick-RIBOmap workflow. FIG. 6B shows in situ sequencing of transcriptional states in thick tissue blocks: The primer, featuring a flanking linker sequence at its 5′ end, is covalently crosslinked (rhombus) to an acrydite-modified oligonucleotide adapter. This crosslinking occurs through a photo-crosslinking reaction between ^{CNV}K and pyrimidines via a [2+2] cycloaddition upon UV-A irradiation (366 nm). Following the preparation of thick tissue slices, the adapter-primer (black) complex and padlock (black) probes with unique gene identifiers hybridize to intracellular mRNAs (dashed line) within the intact tissue. The probe set is copolymerized with acrylamide, forming a DNA-gel hybrid (wavy lines) through the adapter's functionalized acrylic group, followed by the removal of unbound lipids and proteins. Subsequently, enzymatic ligation and rolling circle amplification (RCA) construct in situ cDNA amplicons. These cDNA amplicons are further anchored into the hydrogel network via hydrogel re-embedding. Barcodes on the unique gene identifiers are read out via cyclic in situ sequencing with error reduction by dynamic annealing and ligation (SEDAL). This comprehensive quantification of RNA enables the elucidation of gene expression patterns and the identification of cell types within the native 3D tissue context. FIG. 6C (left) provides schematics and representative fluorescent tissue images of negative and positive control experiments. Using a photocrosslinked 5′ acrydite adapter produces equivalent results to direct 5′ acrydite modification of the primer, both surpassing the performance of adapter-primer hybridization alone and hydrogel physical retention. FIG. 6C (right) shows quantification of cell images showing the average amplicon reads per cell (n=4 images per condition). Gray: DNA amplicon. Black: DAPI. Student's t-test, **** P<0.0001. Data shown as mean±standard deviation. FIG. 6D (Left) provides schematics and representative fluorescent tissue images of 6 rounds of sequencing with and without cDNA re-embedding. In the absence of cDNA re-embedding, BSPEG is used to crosslink cDNA. This results in background accumulation and reduced cDNA detection efficiency. FIG. 6D (right) shows quantification of cell images showing the average amplicon retention rate after 6 rounds of sequencing (n=4 images per condition). Student's t-test, **** P<0.0001. Data shown as mean±standard deviation. Fluorescent images show Ch1 to Ch4 (color-coded channels for barcode decoding) and cell nuclei in mouse brain slices. FIG. 6E provides representative raw fluorescent tissue images across 200 μm and quantification of DNA amplicon signal intensity at different tissue depths.

FIG. 6F shows thick-RIBOmap probe design: Primer (black) and padlock (black) probes with unique gene identifiers hybridize to intracellular mRNAs (dashed line), while splint probes bind to the 18S rRNA of ribosomes. Splint probes serve as templates for proximity ligation and circularization of padlock probes. Both the primer and splint probe feature a flanking linker sequence at their 5′ end and are covalently crosslinked (rhombus) to an acrydite-modified oligonucleotide adapter.

FIGS. 7A-7D show spatial single-cell transcriptomic and translatomic profiling of 1,017 genes in thick mouse brain slices. FIGS. 7A and 7B provide Uniform Manifold Approximation and Projection (UMAP) plot visualizations of transcriptional and translational profiles of 362,704 cells collected from mouse coronal hemibrains using FUSEmap (FIG. 7A) and Harmony integration (FIG. 7B). Surrounding diagrams display 137 subclusters derived from 19 main clusters. FIG. 7C provides a confusion matrix of cell type labels obtained from FUSEmap and Combat Harmony integration, visualizing cell types with more than 100 cells in the sample. FIG. 7D provides 3D molecular cell-type maps derived from thick-STARmap (left) and thick-RIBOmap (right) across adjacent 150-μm thick sections from the mouse hemisphere. Each dot represents one cell, colored by subcluster using the same color code as in FIG. 7A.

FIGS. 8A-8C show the morphological features of transcriptomic types. FIG. 8A shows that thick-STARmap combined with Tetbow enables simultaneous profiling of gene expression and neuron morphologies. AAV-PHP.eB delivers vectors encoding fluorescent proteins and the tTA expression vector. Following tissue sectioning and embedding of probe sets into hydrogel, Tetbow fluorescent proteins and DAPI are imaged. After protein digestion, cDNA amplicons are constructed and sequenced. DAPI co-staining serves as a fiducial marker for image registration, enabling the identification of Tetbow neurons by molecular subtype. FIG. 8B (left) shows volume rendering of neurons in the hippocampus and thalamus labeled with Tetbow. Neurons exhibit unique colors generated by the stochastic and combinatorial expression of three fluorescent proteins (tdTomato, EYFP, and mTurquoise2), enabling the high-resolution identification and differentiation of individual neurons. FIG. 8B (right) show a zoom-in view of volume rendering of mouse cortical pyramidal neurons labeled with Tetbow. FIG. 8C shows representative individual morphological reconstructions of 30 transcriptome-defined subtypes of excitatory and inhibitory neurons. These reconstructions illustrate the distinct morphologies associated with each neuronal subtype, providing insights into the structural diversity within the neural network.

FIGS. 9A-9D show the mapping of cell-cell interactions in human cSCC. FIG. 9A shows UMAP plot visualization of transcriptional profiles of 51,471 cells, integrated using Harmony with a published cSCC scRNA-seq dataset. Cells are color-coded according to their cell-type identity. FIG. 9B provides a dot plot illustrating the top differentially expressed marker genes for each major cluster. The color scale represents the log 2 fold change in gene expression compared to the mean gene expression values across all cells. The dot size indicates the percentage of cells expressing the genes within each major cell type. FIG. 9C provides 3D molecular cell-type maps generated from thick-STARmap, using the same color coding as in FIG. 9A and a zoomed-in view of the interaction between Langerhans cells and TSK cells within a mesh graph of physically neighboring cells. Each cell is depicted as a spot colored according to its main cell type, with physically neighboring cells connected by edges. FIG. 9D shows 3D cell-cell adjacency quantified by the normalized number of edges between pairs of cell types.

FIGS. 10A-10E show the optimization of probe crosslinking. FIG. 10A shows representative fluorescent imaging illustrating probe anchoring efficiency in a hydrogel matrix with various adapter-primer ratios. FIG. 10B shows 15% TBE-Urea gels demonstrating UV crosslinking efficiency with varying adapter-primer molar ratios. CNVK- and acrydite-containing adapter used for UV crosslinking is [5Acryd]GCTA [cnvK]ATACGTCGTACTGGTAGG [Inv-dT](24 nt) (SEQ ID NO: 1). Primer used is 58 bp ssDNA with a 24 bp flanking liner at the 5′ end. M, Marker: IDT ssDNA 20/100 Ladder. FIG. 10C shows quantification of cell images showing the average amplicon reads per cell (n=4 images per condition). Data presented as mean±standard deviation. FIG. 10D provides representative fluorescent imaging demonstrating probe anchoring efficiency with and without the use of the VA-044 thermal initiator. FIG. 10E shows quantification of cell images showing the average amplicon reads per cell (n=4 images per condition). Data presented as mean±standard deviation.

FIGS. 11A-11D show optimization of the re-embedding strategy. FIG. 11A shows the mechanism of cDNA crosslinking using hydrogel re-embedding. Amine-modified nucleotides were incorporated into the rolling-circle amplification reaction. Methacrylic acid N-hydroxysuccinimide ester (MA-NHS) enables rapid conjugation to nucleophilic groups on the amplicons via its NHS ester under mild conditions. These functionalized methacrylamide moieties are then integrated into the hydrogel, effectively immobilizing the cDNA amplicons.

FIG. 11B shows the mechanism of cDNA crosslinking using bis-succinimide ester-activated PEG (BSPEG). Amine-modified nucleotides were incorporated into the rolling-circle amplification reaction followed by BSPEG crosslinking, where the NHS esters of BSPEG react with the amino groups on the amplicons. FIG. 11C shows the mechanism of cDNA crosslinking using click chemistry. Azide and alkyne groups were incorporated during the RCA process, followed by the addition of copper to catalyze the azide-alkyne cycloaddition, forming a stable triazole ring as a crosslinking method. FIG. 11D shows representative fluorescent imaging demonstrating sequencing signal-to-noise ratio using different cDNA crosslinking strategies. BSPEG and click chemistry crosslinking result in higher background noise compared to hydrogel re-embedding after several rounds of sequencing. Additionally, the incorporation of azide and alkyne moieties during RCA significantly reduced amplification efficiency, leading to fewer amplicons.

FIGS. 12A-12G show the comparison of spatial translatome and transcriptome in the mouse brain and cell-cell adjacency analysis. FIG. 12A provides a heatmap showing the gene clustering using the RIBOmap and STARmap results by cell type. FIG. 12B shows visualization of enriched GO terms within each gene module, categorized and color-coded by module. In the enrichment map, nodes represent enriched GO terms, with the size of each node reflecting the number of genes associated with that term. Edges between nodes indicate shared genes among the GO terms. FIG. 12C shows processes read percentages of individual translating genes with genes rank-ordered based on their processes reads percentage. FIG. 12D shows enriched GO terms for processes-enriched and somata-enriched translating genes.

FIG. 12E shows the spatial translation map of representative processes-enriched and somata-enriched translation genes in the hippocampus region. FIGS. 12F and 12G shows distributions of the nearest-neighbor distances from cells in individual inhibitory neuronal subclasses to cells in the same subclass (“to self”) or other subclasses (“to other”). Density: normalize such that the total area of the histogram equals 1.

FIGS. 13A-13D show cell-cell adjacency analysis in 2D and 3D. FIGS. 13A and 13B show quantification of cell-cell adjacency in 3D (FIG. 13A) and 2D (FIG. 13B) by the normalized number of edges between pairs of cell types. The 2D analysis is performed by projecting 15 μm (~1 cell layer) slices along the z-axis, taken within the same 3D volume as shown in FIG. 9. The 3D analysis reveals stronger cell-cell adjacency enrichment. FIGS. 13C and 13D show that the 3D analysis detects stronger cell-cell interactions because the number of connected cells (edges of a given cell in the mesh graph via Delaunay triangulation) is greater than in 2D. The 2D nearest-neighbor distances cannot accurately represent the 3D cellular environment.

### DEFINITIONS

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them unless specified otherwise.

The terms “administer,” “administering,” and “administration” refer to implanting, absorbing, ingesting, injecting, inhaling, or otherwise introducing a treatment or therapeutic agent, or a composition of treatments or therapeutic agents, in or on a subject.

The term “amplicon” as used herein refers to a nucleic acid (e.g., RNA or DNA) that is the product of an amplification reaction (i.e., the production of one or more copies of a genetic fragment or target sequence) or replication reaction. Amplicons can be formed artificially using, for example, PCR or other polymerization reactions. The term “concatenated amplicons” refers to multiple amplicons that are joined together to form a single nucleic acid molecule. Concatenated amplicons can be formed, for example, by rolling circle amplification (RCA), in which a circular oligonucleotide is amplified to produce multiple linear copies of the oligonucleotide as a single nucleic acid molecule comprising multiple amplicons that are concatenated.

The term “biomolecule” or “biological molecule” refers to any substance produced by cells or living organisms and includes carbohydrates, lipids, nucleic acids, proteins, and vitamins. In some embodiments, the methods described herein involve a step of tissue digestion, followed by the clearance of unbound biomolecules, wherein unbound lipids and proteins are removed from the one or more hydrogel-embedded amplicons.

The term “cDNA” refers to DNA that is derived from (e.g., by reverse transcription) and complementary to an RNA template (e.g., an mRNA template or an rRNA template).

A “cell,” as used herein, may be present in a population of cells (e.g., in a tissue, a sample, a biopsy, an organ, or an organoid). In some embodiments, a population of cells is composed of a plurality of different cell types. Cells for use in the methods and systems of the present disclosure can be present within an organism, a single cell type derived from an organism, or a mixture of cell types. Included are naturally occurring cells and cell populations, genetically engineered cell lines, cells derived from transgenic animals, cells from a subject, etc. Virtually any cell type and size can be accommodated in the methods and systems described herein. In some embodiments, the cells are mammalian cells (e.g., complex cell populations such as naturally occurring tissues). In some embodiments, the cells are from a human. In certain embodiments, the cells are collected from a subject (e.g., a human) through a medical procedure, such as a biopsy. Alternatively, the cells may be a cultured population (e.g., a culture derived from a complex population, or a culture derived from a single cell type where the cells have differentiated into multiple lineages). The cells may also be provided in situ in a tissue sample.

Cell types contemplated for use in the methods and systems of the present disclosure include, but are not limited to, stem and progenitor cells (e.g., embryonic stem cells, hematopoietic stem cells, mesenchymal stem cells, neural crest cells, etc.), endothelial cells, muscle cells, myocardial cells, smooth and skeletal muscle cells, mesenchymal cells, epithelial cells, hematopoietic cells, lymphocytes such as T-cells (e.g., Th1 T cells, Th2 T cells, ThO T cells, cytotoxic T cells) and B cells (e.g., pre-B cells), monocytes, dendritic cells, neutrophils, macrophages, natural killer cells, mast cells, adipocytes, immune cells, neurons, hepatocytes, and cells involved with particular organs (e.g., thymus, endocrine glands, pancreas, brain, neurons, glia, astrocytes, dendrocytes, and genetically modified cells thereof). The cells may also be transformed or neoplastic cells of different types (e.g., carcinomas of different cell origins, lymphomas of different cell types, etc.) or cancerous cells of any kind (e.g., from any of the cancers disclosed herein. In some embodiments, cells of multiple cell types are present within the same sample. In certain embodiments, the cells are from a diseased tissue sample or diseased subject. In certain embodiments, the cells are from a healthy tissue sample or healthy subject. In some embodiments, a cell is from a cell line. Cells of different origins (e.g., ectodermal, mesodermal, and endodermal) are also contemplated for use in the methods and systems of the present disclosure. In some embodiments, the cells are microglia, astrocytes, oligodendrocytes, excitatory neurons, or inhibitory neurons. In some embodiments, the cells are cardiac cells. In certain embodiments, the cells are HeLa cells.

The term “complementary” is used herein to refer to two oligonucleotide sequences (e.g., DNA or RNA) comprising bases that hydrogen bond to one another. The degree of complementarity between two oligonucleotide sequences can vary, from complete complementarity to no complementarity (e.g., 100% complementarity, 99% complementarity, 98% complementarity, 97% complementarity, 96% complementarity, 95% complementarity, 90% complementarity, 85% complementarity, 80% complementarity, or less than 80% complementarity). For example, two oligonucleotide sequences may be only partially complementary to one another (e.g., in the probes described herein, wherein only a portion of the probe is complementary to a nucleotide sequence). In some embodiments, a sequence is complementary to only a portion of another sequence. In some embodiments, a sequence is complementary to another sequence under certain conditions (e.g., certain salt concentrations, pHs, etc.).

The term “click chemistry” refers to a class of simple, atom-economy reactions commonly used for joining two molecular entities of choice. Click chemistry relies on two reaction partners (click handles) that can attach to each other very rapidly and selectively, without the production of any toxic by-products. For example, four major classifications of click reactions have been identified: (1) Cycloadditions—these primarily refer to 1,3-dipolar cycloadditions, but also include hetero-Diels-Alder cycloadditions; (2) Nucleophilic ring-openings—these refer to the openings of strained heterocyclic electrophiles, such as aziridines, epoxides, cyclic sulfates, aziridinium ions, episulfonium ions, etc.; (3) Carbonyl chemistry of the non-aldol type—examples include the formations of ureas, thioureas, hydrazones, oxime ethers, amides, aromatic heterocycles, etc. Carbonyl reactions of the aldol type generally have low thermodynamic driving forces, hence they have longer reaction times and give side products, and therefore cannot be considered click reactions; and (4) Additions to carbon-carbon multiple bonds—examples include epoxidations, aziridinations, dihydroxylations, sulfenyl halide additions, nitrosyl halide additions, and certain Michael additions. In certain embodiments, cycloadditions, particularly the Cu¹-catalyzed Huisgen 1,3-dipolar cycloaddition (HDC) of azides and terminal alkynes to form 1,2,3-triazoles are used to crosslink amplicons and embed them into the hydrogel. Click chemistry is further described in Hein et al., Click chemistry, a powerful tool for pharmaceutical sciences. Pharm. Res. 2008; 25 (10): 2216-30, which is incorporated herein by reference.

The terms “hydrogel” or “hydrogel network” mean a network of polymer chains that are water-insoluble, sometimes found as a colloidal gel in which water is the dispersion medium. In other words, hydrogels are a class of polymeric materials that can absorb large amounts of water without dissolving. Hydrogels can contain over 99% water and may include natural or synthetic polymers, or a combination thereof. Hydrogels also possess a degree of flexibility very similar to natural tissue, due to their significant water content. A detailed description of suitable hydrogels may be found in published U.S. Patent Application Publication No. 2010/0055733, which is incorporated by reference herein. As used herein, the term “hydrogel monomer” refers to hydrophilic monomers, prepolymers, or polymers that can be crosslinked, or “polymerized”, to form a three-dimensional (3D) hydrogel network. In some embodiments, the fixation of the tissue sample in the presence of hydrogel subunits crosslinks the components of the sample to the hydrogel subunits, thereby securing molecular components in place, preserving the tissue architecture and cell morphology.

The term “locked nucleic acid” (LNA) refers to a modified RNA nucleotide in which the ribose moiety is modified with an extra bridge connecting the 2′ oxygen and 4′ carbon. In some embodiments, the use of LNA nucleotides enhances biostability and improves thermodynamics of hybridization to RNA. In some embodiments, LNA nucleotides can be mixed with DNA and/or RNA residues in an oligonucleotide. In some embodiments, an oligonucleotide consists of LNA nucleotides.

The terms “polynucleotide,” “nucleotide sequence,” “nucleic acid,” “nucleic acid molecule,” “nucleic acid sequence,” and “oligonucleotide” refer to a series of nucleotide bases (also called “nucleotides”) in DNA and RNA and mean any chain of two or more nucleotides. The polynucleotides can be chimeric mixtures or derivatives or modified versions thereof, and single-stranded or double-stranded. The oligonucleotide can be modified at the base moiety, sugar moiety, or phosphate backbone, for example, to improve stability of the molecule, its hybridization parameters, etc. Polynucleotides can also include modified nucleotides, e.g., LNA.

The term “profiling” (in reference to profiling RNA expression and/or translation in the methods provided herein) refers to determining the expression and/or translation pattern of multiple (potentially thousands of) genes at once to create a global picture of cellular function. An RNA expression profile includes information about which genes are and are not expressed by a particular cell at a particular time point. An RNA expression profile also includes information about the levels at which genes are expressed in the cell. An RNA translation profile includes information about which RNAs are actively being translated in a cell, and the levels at which RNAs actively being translated are being expressed in the cell. The RNA expression and/or translation profile of a cell can be used, for example, to determine the cell type of the cell, the stage of cell division the cell is at, whether the cell is from a diseased or healthy tissue, or how the cell responds to treatment with a particular agent.

A “protein,” “peptide,” or “polypeptide” comprises a polymer of amino acid residues linked together by peptide bonds. The term refers to proteins, polypeptides, and peptides of any size, structure, or function. Typically, a protein will be at least three amino acids long. A protein may refer to an individual protein or a collection of proteins. Proteins may contain only natural amino acids, although non-natural amino acids (i.e., compounds that do not occur in nature but that can be incorporated into a polypeptide chain) and/or amino acid analogs as are known in the art may alternatively be employed. Also, one or more of the amino acids in a protein may be modified, for example, by the addition of a chemical entity such as a carbohydrate group, a hydroxyl group, a phosphate group, a farnesyl group, an isofarnesyl group, a fatty acid group, a linker for conjugation or functionalization, or other modification. A protein may also be a single molecule or may be a multi-molecular complex. A protein may be a fragment of a naturally occurring protein or peptide. A protein may be naturally occurring, recombinant, synthetic, or any combination of these. A protein may also be a therapeutic protein administered as a treatment for a disease or disorder (e.g., one that is associated with a change in the RNA expression and/or translation profile of a cell taken from a subject). In certain embodiments, the protein is an antibody, or an antibody variant (including antibody fragments).

A “research agent,” as used herein, may refer to any small molecules, nucleic acids, peptides, proteins, lipids, carbohydrates, or any reagents used to perturb a biological system for research purposes.

A “transcript” or “RNA transcript” is the product resulting from RNA polymerase-catalyzed transcription of a DNA sequence. When the RNA transcript is a complementary copy of a DNA sequence, it is referred to as the primary transcript, or it may be an RNA sequence derived from post-transcriptional processing of the primary transcript and is then referred to as the mature RNA. “Messenger RNA (mRNA)” refers to the RNA that is without introns and can be translated into a polypeptide by the cell.

The term “sample” or “biological sample” refers to any sample including tissue samples (such as tissue sections, surgical biopsies, and needle biopsies of a tissue); cell samples; or cell fractions, fragments, or organelles (such as obtained by lysing cells and separating the components thereof by centrifugation or otherwise). Other examples of biological samples include, but are not limited to, blood, serum, urine, semen, fecal matter, cerebrospinal fluid, interstitial fluid, mucous, tears, sweat, pus, biopsied tissue (e.g., obtained by a surgical biopsy or needle biopsy), nipple aspirates, milk, vaginal fluid, saliva, swabs (such as buccal swabs), or any material containing biomolecules that is derived from a first biological sample. In some embodiments, a biological sample is a surgical biopsy taken from a subject, for example, a biopsy of any of the tissues described herein. In certain embodiments, a biological sample is a tumor biopsy. In some embodiments, the sample is brain tissue. In some embodiments, the tissue is cardiac tissue. In some embodiments, the sample is epithelial tissue, connective tissue, muscular tissue, or nervous tissue. In some embodiments, the sample is tissue from the central nervous system (e.g., brain). In some embodiments, the tissues used in the methods described herein come from such a sample or biological sample. In some embodiments, the sample is brain tissue. In some embodiments, the sample is brain tissue for imaging neurons (e.g., by TetBow).

A “subject” to which administration is contemplated refers to a human (i.e., male or female of any age group, e.g., pediatric subject (e.g., infant, child, or adolescent) or adult subject (e.g., young adult, middle-aged adult, or senior adult)) or non-human animal. In some embodiments, the non-human animal is a mammal (e.g., primate (e.g., cynomolgus monkey or rhesus monkey) or mouse). The term “patient” refers to a subject in need of treatment of a disease. In some embodiments, the subject is human. In some embodiments, the patient is human. The human may be a male or female at any stage of development. A subject or patient “in need” of treatment of a disease or disorder includes, without limitation, those who exhibit any risk factors or symptoms of a disease or disorder. In some embodiments, a subject is a non-human experimental animal (e.g., a mouse, rat, dog, pig, or non-human primate).

The term “therapeutic agent,” as used herein, refers to any agent that can be used to treat a disease or disorder, or reduce or alleviate the symptoms of a disease or disorder. In some embodiments, the therapeutic agent is a small molecule, a protein, a peptide, a nucleic acid, a lipid, or a carbohydrate. In some embodiments, the therapeutic agent is a known drug and/or an FDA-approved drug. In certain embodiments, the protein is an antibody. In certain embodiments, the protein is an antibody variant. In certain embodiments, the protein is a receptor, or a fragment or variant thereof. In certain embodiments, the protein is a cytokine. In certain embodiments, the nucleic acid is an mRNA, an antisense RNA, an miRNA, an siRNA, an RNA aptamer, a double stranded RNA (dsRNA), a short hairpin RNA (shRNA), or an antisense oligonucleotide (ASO).

A “therapeutically effective amount” of a treatment or therapeutic agent is an amount sufficient to provide a therapeutic benefit in the treatment of a condition or to delay or minimize one or more symptoms associated with the condition. A therapeutically effective amount of a treatment or therapeutic agent means an amount of the therapy, alone or in combination with other therapies, that provides a therapeutic benefit in the treatment of the condition. The term “therapeutically effective amount” can encompass an amount that improves overall therapy, reduces or avoids symptoms, signs, or causes of the condition, and/or enhances the therapeutic efficacy of another therapeutic agent.

As used herein, a “tissue” is a group of cells and their extracellular matrix from the same origin. Together, the cells carry out a specific function. The association of multiple tissue types together forms an organ. The cells may be of different cell types. In some embodiments, a tissue is an epithelial tissue. Epithelial tissues are formed by cells that cover an organ surface (e.g., the surface of the skin, airways, soft organs, reproductive tract, and inner lining of the digestive tract). Epithelial tissues perform protective functions and are also involved in secretion, excretion, and absorption. Examples of epithelial tissues include, but are not limited to, simple squamous epithelium, stratified squamous epithelium, simple cuboidal epithelium, transitional epithelium, pseudostratified epithelium, columnar epithelium, and glandular epithelium. In some embodiments, a tissue is a connective tissue. Connective tissues are fibrous tissues made up of cells separated by non-living material (e.g., an extracellular matrix). Connective tissues provide shape to organs and hold organs in place. Connective tissues include fibrous connective tissue, skeletal connective tissue, and fluid connective tissue. Examples of connective tissues include, but are not limited to, blood, bone, tendon, ligament, adipose, and areolar tissues. In some embodiments, a tissue is a muscular tissue. Muscular tissue is an active contractile tissue formed from muscle cells. Muscle tissue functions to produce force and cause motion. Muscle tissue includes smooth muscle (e.g., as found in the inner linings of organs), skeletal muscle (e.g., as typically attached to bones), and cardiac muscle (e.g., as found in the heart, where it contracts to pump blood throughout an organism). In some embodiments, a tissue is a nervous tissue. Nervous tissue includes cells comprising the central nervous system and peripheral nervous system. Nervous tissue forms the brain, spinal cord, cranial nerves, and spinal nerves (e.g., motor neurons).

The term “transcriptome” refers to all of the RNA transcripts (whether coding or non-coding) that are expressed in a cell or population of cells, a sample (e.g., a tissue sample), tissue, organ, or in a subject. In some embodiments, the methods, probes, compositions, systems, uses, and kits provided herein are useful for studying and/or profiling the transcriptome.

The term “translatome” refers to all of the open reading frames (i.e., DNA sequences that fall between start and stop codons) that are actively being transcribed in a particular cell, sample, tissue, organ, or organism. In some embodiments, the methods, probes, compositions, systems, uses, and kits provided herein are useful for studying and/or profiling the translatome.

The terms “treatment,” “treat,” and “treating” refer to reversing, alleviating, delaying the onset of, or inhibiting the progress of a disease described herein. In some embodiments, treatment may be administered after one or more signs or symptoms of the disease have developed or have been observed (e.g., prophylactically or upon suspicion or risk of disease). In other embodiments, treatment may be administered in the absence of signs or symptoms of the disease. For example, treatment may be administered to a susceptible subject prior to the onset of symptoms (e.g., in light of a history of symptoms in the subject, or family members of the subject). Treatment may also be continued after symptoms have resolved, for example, to delay or prevent recurrence. In some embodiments, treatment may be administered after using the methods disclosed herein and observing a change in the RNA expression or translation profile in a cell or tissue in comparison to a healthy cell or tissue.

Throughout the present disclosure, when a range of values is listed, it is intended to encompass each value and sub-range within the range. Where ranges are given, endpoints are included.

### DETAILED DESCRIPTION OF CERTAIN EMBODIMENTS

The aspects described herein are not limited to specific embodiments, systems, compositions, methods, kits, uses, or configurations, and as such can, of course, vary. The terminology used herein is for the purpose of describing particular aspects only and, unless specifically defined herein, is not intended to be limiting.

The present disclosure provides methods, uses, compositions, kits, and systems for profiling RNAs being transcribed and/or translated in a thick tissue sample (e.g., greater than 10-20 microns). The present disclosure also provides methods for diagnosing a disease or disorder in a subject based on a profile of the RNAs being transcribed and/or translated in a tissue sample. Methods of screening for or testing a candidate agent capable of modulating the transcription and/or translation of one or more RNAs are also provided by the present disclosure. The present disclosure also provides methods for treating a disease or disorder in a subject in need thereof. Oligonucleotide probes and sets of oligonucleotide probes, which may be useful for performing the methods described herein, are also provided by the present disclosure, as well as compositions and kits comprising any of the oligonucleotide probes described herein.

Methods for Profiling RNA Expression and/or Translation

In one aspect, the present disclosure provides methods for profiling RNA expression and/or translation in a thick tissue sample. Such methods are useful for profiling RNA expression and translation in a tissue sample that is more than 10-20 μm thick. In some embodiments, the tissue is at least 20, at least 50, at least 100, at least 150, at least 200, at least 250, at least 300, or at least 350 μm thick. In the methods disclosed herein, a tissue sample may be contacted with one or more sets of oligonucleotide probes, wherein the probes hybridize to RNAs within the tissue sample. One or more of the probes comprise at least one crosslinking moiety (for example, an acrydite moiety, e.g., at the 5′ end of the probe), allowing the probes and tissue sample to be embedded in a hydrogel matrix prior to any subsequent steps in the method. Following tissue digestion and the removal of unbound biomolecules (e.g., lipids and proteins), cDNA amplicons with unique gene identifiers are synthesized via enzymatic ligation and rolling circle amplification (RCA). The one or more concatenated amplicons may then be embedded in a polymeric matrix and sequenced using SEDAL sequencing to determine the quantification and location of the transcripts and their respective translation within intact tissue samples.

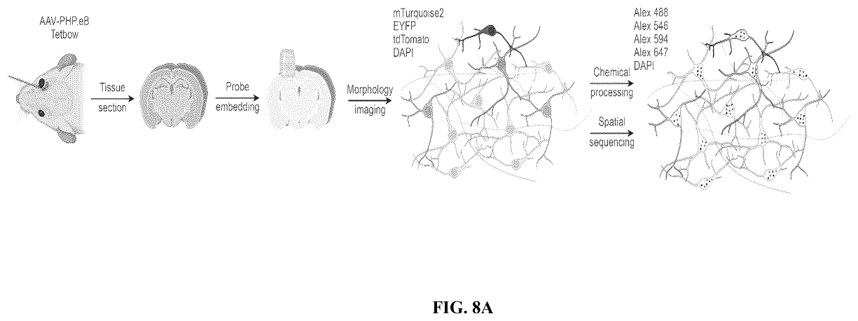

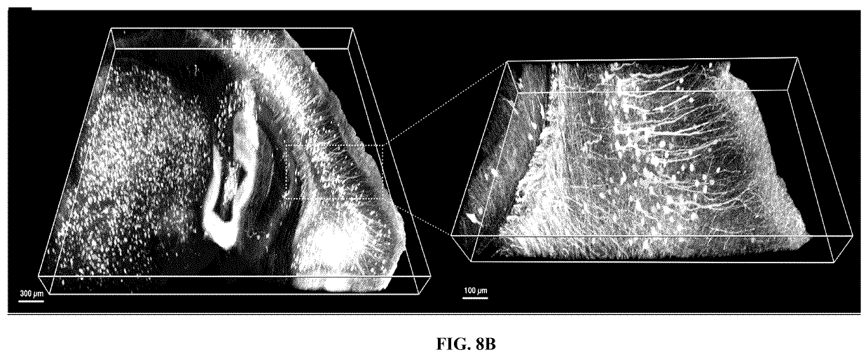

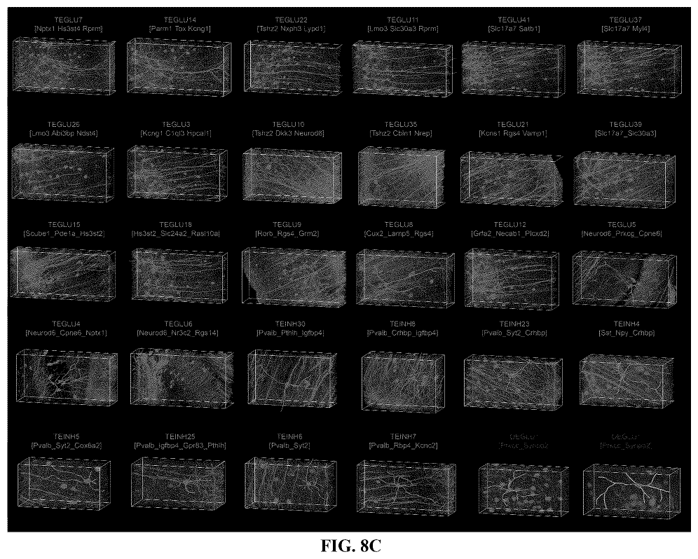

In certain embodiments, the methods disclosed herein comprise a step of Tetbow labeling, which is a stochastic multicolor labeling method that uses a tetracycline-operator system (Tetbow). It has been demonstrated that systemically delivered Adeno-associated viruses (AAVs) allow a more uniform distribution of labeled cells and color diversity. Thus, the present disclosure describes the use of an AAV-PHP.eB variant to co-administer three separate vectors encoding the fluorescent proteins (e.g., mTurquoise2, Enhanced Yellow Fluorescent Protein (EYFP), and Tandem dimer Tomato (tdTomato)) along with a tTA expression vector. The AAV-PHP.eB variant has been demonstrated to transduce the central nervous system, thereby facilitating widespread fluorescent protein expression across the brain (see FIG. 8B). Use of Tetbow is described in Sakaguchi et al., Bright multicolor labeling of neuronal circuits with fluorescent proteins and chemical tags 2018. eLife, 7, e40350, which is incorporated herein by reference. The use of Tetbow enables comprehensive and high-resolution mapping of intermingled neurons in situ by tagging individual neurons with stochastic combinations of different fluorescent proteins.

In some embodiments, the present disclosure provides methods for profiling RNA expression and/or translation in a tissue sample comprising the steps of:
a) contacting the tissue sample with 1) one or more sets of oligonucleotide probes, wherein each set of oligonucleotide probes comprises a first probe and a second probe, and 2) a third oligonucleotide probe, wherein
   i) the first probe comprises a portion that is complementary to at least a portion of the third probe, a portion that is complementary to an RNA of interest, and a portion that is complementary to a portion of the second probe;
   ii) the second probe comprises a portion that is complementary to a portion of the first probe, a portion that is complementary to the RNA of interest, and an oligonucleotide barcode sequence; and
   iii) the third probe comprises a portion that is complementary to the first probe and at least one crosslinking moiety;
b) co-polymerizing the one or more sets of probes, the third probe, and a polymeric matrix to form an oligonucleotide-polymeric matrix hybrid, wherein the first probe is hybridized to the RNA of interest;
c) removing unbound biomolecules from the oligonucleotide-polymeric matrix hybrid;
d) ligating the 5′ end and the 3′ end of the second probe together to produce a circular oligonucleotide;
e) performing rolling circle amplification to amplify the circular oligonucleotide using the first probe as a primer, thereby producing one or more concatenated amplicons;
f) embedding the one or more concatenated amplicons in the polymeric matrix; and
g) sequencing the concatenated amplicons, or a portion thereof, embedded in the polymeric matrix to determine the identity and location of each RNA of interest in the tissue sample.

The present disclosure contemplates any arrangement of the portions of the first probe. In some embodiments, each first probe comprises the structure 5′-[portion complementary to portion of third probe]-[portion complementary to RNA of interest]-[portion complementary to portion of second probe]-3′, wherein each instance of]-[represents an optional linker (e.g., a nucleotide linker). In some embodiments, “]-[” represents a direct linkage between the two portions of the first probe (i.e., a phosphodiester bond). In some embodiments, the first probes described herein may comprise standard nucleotides (e.g., DNA, RNA), or some of the standard nucleotides may be substituted for any modified nucleotides known in the art. In some embodiments, portions or all of the first probes described herein may comprise modified nucleotides (e.g., LNA nucleotides) or nucleotide analogs.

In some embodiments, the portion of the first probe that is complementary to a portion of the third probe comprises the sequence CCTACCAGTACGACGTATTTAGCAA (SEQ ID NO: 2) at its 5′ end (also referred to herein as the “flanking linker sequence”), or a sequence at least 80%, at least 85%, at least 90%, at least 95%, or at least 99% identical to CCTACCAGTACGACGTATTTAGCAA (SEQ ID NO: 2).

In some embodiments, the portion of the first probe that is complementary to at least a portion of the third probe is 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 nucleotides in length. In certain embodiments, the portion of the first probe that is complementary to at least a portion of the third probe is about 24 nucleotides in length. In some embodiments, the portion of the first probe that is complementary to an RNA of interest and the portion of the first probe that is complementary to a portion of the second probe together comprise 40, 41, 42, 43, 44, 45, 46, 47, 48, 49, 50, 51, 52, 53, 54, 55, 56, 57, 58, 59, 60, 61, 62, 63, 64, 65, 66, 67, 68, 69 70, 71, 72, 73, 74, 75, 76, 77, 78, 79, or 80 nucleotides in length. In some embodiments, the portion of the first probe that is complementary to an RNA of interest and the portion of the first probe that is complementary to a portion of the second probe together comprise about 58 nucleotides in length.

The present disclosure contemplates any arrangement of the portions of the second probe. In some embodiments, the second probe in the set of probes comprises the structure 5′-[portion complementary to portion of first probe]-[portion complementary to RNA of interest]-[oligonucleotide barcode sequence]-[portion complementary to portion of first probe]-3′, wherein each instance of]-[represents an optional linker (e.g., a nucleotide linker). In some embodiments, “]-[” represents a direct linkage between the two portions of the second probe (i.e., a phosphodiester bond). In some embodiments, the second probes described herein may comprise standard nucleotides (e.g., DNA, RNA), or some of the standard nucleotides may be substituted for any modified nucleotides known in the art. In some embodiments, portions of the second probes described herein may comprise modified nucleotides (e.g., LNA nucleotides) or nucleotide analogs.

In some embodiments, the second probe used in the methods described herein includes an oligonucleotide barcode sequence made up of a specific sequence of nucleotides. In some embodiments, the oligonucleotide barcode sequence is 5, 6, 7, 8, 9, 10, 11, 12, 13, 14, or 15 nucleotides in length. In some embodiments, the barcodes of the second probe may comprise gene-specific sequences used to identify RNAs of interest during sequencing as described further below (i.e., each barcode sequence is associated with a specific gene or transcript). The use of the barcodes on probes analogous to those described herein is further described in, for example, International Patent Application Publication No. WO 2019/199579, published Oct. 17, 2019, and Wang et al., Science 2018, 361, 380, both of which are incorporated herein by reference.

The present disclosure contemplates any arrangement of the portions of the third probe. In some embodiments, the third probe in the set of probes comprises the structure: 5′-[first crosslinking moiety]-[portion complementary to first probe]-3′. In certain embodiments, the third probe comprises the sequence:

5′-[first crosslinking moiety]GCTA [nucleoside analog comprising second crosslinking moiety]ATACGTCGTACTGGTAGG [Inverted-dT]-3′ (SEQ ID NO: 3). In certain embodiments, the third probe comprises the sequence: 5′-[Acrydite]GCTA [CNVK3]ATACGTCGTACTGGTAGG [Inverted-dT]-3′ (SEQ ID NO: 4). Each instance of]-[in the third probe independently represents an optional linker (e.g., a nucleotide linker). In some embodiments, “]-[” represents a direct linkage between the two portions of the third probe (i.e., a phosphodiester bond). In some embodiments, the third probes described herein may comprise standard nucleotides (e.g., DNA, RNA), or some of the standard nucleotides may be substituted for any modified nucleotides known in the art. In some embodiments, portions of the third probes described herein may comprise modified nucleotides (e.g., LNA nucleotides) or nucleotide analogs. In some embodiments, the third probe is 15, 16, 17, 18, 19, 20, 21, 22, 23, 24, 25, 26, 27, 28, 29, or 30 nucleotides in length. In certain embodiments, the third probe is about 24 nucleotides in length.

In certain embodiments, the portion of the third probe that is complementary to the first probe comprises one or more nucleoside analogs comprising a second crosslinking moiety (in addition to the crosslinking moiety at the 5′ end of the probe). The second crosslinking moiety may be useful, for example, for stabilizing the complex formed between the first and third probes via the formation of a covalent bond. Any nucleoside analogs comprising a crosslinking moiety may be used in the third probe, and the methods of the present disclosure also contemplate the use of crosslinking mechanisms, such as, but not limited to, the use of cyano-modified oligonucleotides, furan-modified oligonucleotides, and psoralen. The present disclosure further recognizes crosslinks can be formed by covalent cyclobutene pyrimidine dimer (CPD) bonds induced via ultraviolet irradiation, the use of reductive amination reactions between an abasic site and the non-canonical nucleobase 2-aminopurine, and the use of oxidation reactions between a singlet oxygen and a modified nucleotide (e.g., an analogue of thymidine). In some embodiments, the nucleoside analog comprises a cyano moiety. Nucleoside analogs comprising cyano moieties useful for forming nucleic acid crosslinks are described, for example, in Yoshimura and Fujimoto, Org. Lett. 2008, 10 (15), 3227-3230, which is incorporated herein by reference. In some embodiments, the nucleoside analog comprises a furan moiety (which may, for example, be oxidized to form a 4-oxo-enal derivative, facilitating formation of a crosslink between two nucleic acids as described in Stevens et al., Nucleic Acids Research 2009, 37 (5), 1555-1565, which is incorporated herein by reference). In some embodiments, proximal thymidines on the first and the third probes are used to form a crosslink, for example, by exposing the thymidines to UV irradiation. Further methods of crosslinking two nucleic acid molecules are described, for example, in Hong and Greenberg, J. Am. Chem. Soc. 2005, 127 (30), 10510-10511 and Nejad et al., ACS Chem. Biol. 2019, 14 (7), 1481-1489, each of which is incorporated herein by reference. In some embodiments, the nucleoside analog comprises a thiol group. In some embodiments, the nucleoside analog is 3-cyanovinylcarbazole (CNVK3), Psoralen C2 phosphoramidite, 6-thio-dG, 4-thio-dT, or 4-thio-dU. In certain embodiments, the nucleotide analog is 3-cyanovinylcarbazole (CNVK3). In some embodiments, the CNVK3-containing third probe is photo crosslinked to a pyrimidine base of the first probe upon ultraviolet (UV) irradiation (e.g., at a wavelength of about 360-370 nm, for example, about 366 nm). In some embodiments, the reaction time is about 5, about 10, about 15, about 20, about 25, about 30, or more than 30 minutes. In certain embodiments, the reaction time is about 10 minutes. In some embodiments, the ratio of the third probe to the first probe added to the tissue sample in the methods provided herein is at least 3:1, at least 4:1, at least 5:1, at least 6:1, at least 7:1, at least 8:1, at least 9:1, or at least 10:1 during the crosslinking reaction. In certain embodiments, the ratio of the third probe to the first probe is about 5:1 during the crosslinking reaction.

In some embodiments, at least one crosslinking moiety of the third probe comprises acrydite. In certain embodiments, acrydite comprises the structure

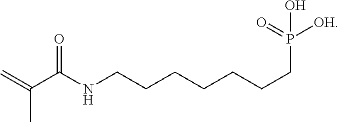 [SMILES: C=C(C)C(=O)NCCCCCCCP(=O)(O)O]

The methods of the present disclosure also contemplate the use of other comparable mechanisms for further functionalizing the third probe. For example, in addition to incorporating a 5′ acrylamide modification, amine-modified nucleoside analogs can be introduced into the design of the third probe. The amine moieties can, e.g., be treated with acrylic acid N-hydroxysuccinimide esters, further functionalizing the adapter with additional acrylate groups that can facilitate crosslinking.

In some embodiments, third probe comprises a polymerization blocker at its 3′ end. The polymerization blocker can be, for example, any chemical moiety that prevents a polymerase from using the third probe as a primer for polymerization. For example, the polymerization blocker may be a nucleic acid residue comprising a blocked 3′ hydroxyl group (e.g., comprising an oxygen protecting group on the 3′ hydroxyl group). In some embodiments, the polymerization blocker comprises an inverted nucleic acid residue. In some embodiments, the polymerization blocker is an inverted adenosine, thymine, cytosine, guanosine, or uridine residue. In certain embodiments, the polymerization blocker is an inverted thymine residue. In some embodiments, the inverted nucleotide may help ensure that the third probe is not used as a primer during rolling circle amplification.

In some embodiments, the set of probes comprises a fourth probe for profiling expression of RNAs that are actively being translated in a tissue sample. The present disclosure contemplates any arrangement of the portions of the fourth probe. The fourth probe in the set of probes comprises a portion complementary to the third probe, a portion complementary to at least a portion of a ribosomal RNA (rRNA), and a portion complementary to the second probe. In some embodiments, the portion of the fourth probe that is complementary to at least a portion of the rRNA is complementary to at least a portion of an 18S rRNA. In some embodiments, the fourth probe comprises the structure 5′-[portion complementary to third probe]-[portion complementary to rRNA]-[portion complementary to portion of second probe]-3′, wherein “]-[” represents an optional linker (e.g., a nucleotide linker). In some embodiments, “]-[” represents a direct linkage between the two portions of the primer probe (i.e., a phosphodiester bond). In some embodiments, the fourth probes described herein may comprise standard nucleotides (e.g., DNA, RNA), or some of the standard nucleotides may be substituted for any modified nucleotides known in the art. In some embodiments, portions of the fourth probes described herein may comprise modified nucleotides (e.g., LNA nucleotides) or nucleotide analogs. In some embodiments, the portion of the fourth probe that is complementary to at least a portion of an rRNA is 5, 6, 7, 8, 9, 10, 11, 12, 13, 14, 15, 16, 17, 18, 19, 20, 21, 22, 23, 24, 25, 26, 27, 28, 29, or 30 nucleotides in length.

In some embodiments, the portion of the fourth probe that is complementary to the third probe comprises the same sequence as the portion of the first probe that is complementary to the third probe. In some embodiments, the portion of the fourth probe that is complementary to a portion of the third probe comprises the sequence CCTACCAGTACGACGTATTTAGCAA (SEQ ID NO: 2) at its 5′ end (also referred to herein as the “flanking linker sequence”), or a sequence at least 80%, at least 85%, at least 90%, at least 95%, or at least 99% identical to the sequence CCTACCAGTACGACGTATTTAGCAA (SEQ ID NO: 2).

In certain embodiments, the second crosslinking moiety of the third probe is covalently crosslinked to a pyrimidine base of the fourth probe by photo crosslinking (e.g., upon UV irradiation at a wavelength of about 360-370 nm, for example, about 366 nm). In some embodiments, the photocrosslinking is performed for about 5, about 10, about 15, about 20, about 25, about 30, or more than 30 minutes. In certain embodiments, the photocrosslinking is performed for about 10 minutes.

In embodiments comprising the use of a fourth probe for profiling RNAs actively being translated by a ribosome, the second probe may comprise the structure 5′-[portion complementary to portion of fourth probe]-[portion complementary to portion of first probe]-[portion complementary to RNA of interest]-[oligonucleotide barcode sequence]-[portion complementary to portion of fourth probe]-3′.

In the methods described herein, the one or more sets of probes and the tissue sample are embedded into the polymeric matrix prior to tissue digestion. In some embodiments, embedding the probe sets and tissue into the polymeric matrix comprises co-polymerizing the one or more sets of probes, tissue, and polymeric matrix by reacting the crosslinking moiety (e.g., an acrydite-modified oligonucleotide or another nucleotide analog) of the third probe with the polymeric matrix to form an oligonucleotide-polymeric matrix hybrid. In some embodiments, the hydrogel is formed by a polymerization reaction (e.g., reaction of acrylamide and bis-acrylamide). Without wishing to be bound by theory, such a reaction may be initiated by free radicals, which can be generated using a catalyst (for example, ammonium persulfate (APS)) and a stabilizer (for example, TEMED (N,N,N′,N′-tetramethylethylenediamine)). In an exemplary reaction, acrylamide monomers link together to form long polymer chains. Bis-acrylamide serves as a crosslinker that binds these chains together, creating a mesh-like network. The oligonucleotide adapter is incorporated into the hydrogel by a crosslinking moiety (for example, an acrydite group). During the polymerization reaction, the crosslinking moiety at the end of the oligonucleotide adapter can covalently bond with the acrylamide or bis-acrylamide molecules. As the hydrogel forms, the acrydite-modified adapters become covalently crosslinked within the hydrogel. This effectively immobilizes the oligonucleotide adapters within the hydrogel.

In some embodiments, the resulting tissue-polymeric matrix hybrid is subjected to removal of unbound biomolecules (e.g., unbound proteins and/or lipids). In certain embodiments, the removal step comprises performing protein digestion and/or lipid removal. Inclusion of such a step may facilitate, for example, enhancement of enzyme (e.g., ligases and polymerases) penetration into the tissue-polymeric matrix hybrid, ensuring sufficient diffusion of the enzymes throughout the thickness of the tissue sample. In certain embodiments, the tissue-polymeric matrix hybrid is contacted with a protease. For example, the tissue-polymeric matrix hybrid may be contacted with Proteinase K. Protease treatment can be performed over a range of times at a range of temperatures, and over a range of enzyme concentrations that are empirically determined for each cell type or tissue type under investigation. In some embodiments, the tissue-gel hybrids are digested with 0.1, 0.2, 0.3, 0.4, 0.5, 0.6, 0.7, 0.8, 0.9, or 1.0 mg/mL Proteinase K. In some embodiments, protease treatment is performed at approximately 37 °C (e.g., at 30, 31, 32, 33, 34, 35, 36, 37, 38, 39, or 40 °C). In some embodiments, protease digestion is performed for at least 15, at least 16, at least 17, at least 18, at least 19, at least 20, at least 21, at least 22, at least 23, or at least 24 hours (e.g., overnight).

The methods provided herein also comprise a step of ligating the 5′ end and the 3′ end of the second probe together to produce circular DNA molecules for use in rolling circle amplification (RCA). In some embodiments, ligating the 5′ end and the 3′ end of the second probe together further comprises contacting the tissue sample with a DNA ligase.

In some embodiments, following tissue digestion, the step of performing rolling circle amplification to amplify the circular oligonucleotide to produce one or more concatenated amplicons further comprises providing nucleotide analogs modified with reactive chemical groups (e.g., amine modified nucleotides or any nucleotides comprising a nucleophile, such as 5-(3-aminoallyl)-dUTP). Examples of other amine-modified nucleotides include, but are not limited to, a 5-Aminoallyl-dUTP moiety modification, a 5-Propargylamino-dCTP moiety modification, a N6-6-Aminohexyl-dATP moiety modification, or a 7-Deaza-7-Propargylamino-dATP moiety modification. In some embodiments, the step of embedding the one or more concatenated amplicons in a polymeric matrix comprises reacting the amine-modified nucleotides of the one or more concatenated amplicons with a crosslinking agent (e.g., BS(PEG)₉) and co-polymerizing the one or more concatenated amplicons and the polymeric matrix. In other embodiments, the concatenated amplicons are re-embedded in a polymeric matrix with 2% acrylamide, 0.05% bis-acrylamide to enable cDNA amplicon crosslinking in the tissue-hydrogel setting.

In certain embodiments, click chemistry is utilized to embed the one or more concatenated amplicons in a polymeric matrix. For example, an azide (e.g., 5-azidomethyl-dUTP) and an alkyne group (e.g., 5-(3-ethynyl)-dUTP (5-EdUTP)) can be incorporated during the rolling circle amplification process, followed by the addition of copper to catalyze the azide-alkyne cycloaddition, forming a stable triazole ring as a crosslinking method.

The use of various polymeric matrices is contemplated by the present disclosure, and any polymeric matrix in which the one or more concatenated amplicons can be embedded is suitable for use in the methods described herein. In some embodiments, the polymeric matrix is a hydrogel (i.e., a network of crosslinked polymers that are hydrophilic). In some embodiments, the hydrogel is a polyvinyl alcohol hydrogel, a polyethylene glycol hydrogel, a polyacrylate hydrogel, or a polyacrylamide hydrogel. In certain embodiments, the polymeric matrix comprises about 10-20% (e.g., about 15%) TBE-Urea. Such a hydrogel may be prepared, for example, by incubating the sample in a buffer comprising acrylamide and bis-acrylamide, removing the buffer, and incubating the sample in a polymerization mixture (comprising, e.g., ammonium persulfate and tetramethylethylenediamine). Such reagents may also be provided in a kit, e.g., a kit for performing any of the methods described herein, or any of the kits described herein.

The methods disclosed herein also include a step of sequencing the concatenated amplicons, or a portion thereof, embedded in the polymeric matrix using the barcode sequences in the probes. In some embodiments, the step of sequencing comprises performing “sequencing with error-reduction by dynamic annealing and ligation” (SEDAL sequencing), where each barcode sequence is read out using a complementary oligonucleotide probe fused to a fluorophore. SEDAL sequencing is described further in Wang, X. et al., “Three-dimensional intact-tissue sequencing of single-cell transcriptional states.” Science 2018, 361, 380, and International Patent Application Publication No. WO 2019/199579, published Oct. 17, 2019, each of which is incorporated herein by reference.

The oligonucleotide sequencing probes used in the methods described herein (e.g., as used in SEDAL sequencing) may be read out using any suitable imaging technique known in the art. For example, in embodiments where the oligonucleotide sequencing probes comprise a fluorophore, the fluorophore may be read out using imaging to sequence and identify each RNA. In some embodiments, imaging comprises fluorescent imaging. In certain embodiments, imaging comprises confocal microscopy. In certain embodiments, imaging comprises epifluorescence microscopy. In certain embodiments, two rounds of imaging are performed. In certain embodiments, three rounds of imaging are performed. In certain embodiments, four rounds of imaging are performed. In certain embodiments, five or more rounds of imaging are performed. In certain embodiments, a sample may be stained for imaging. In certain embodiments, the sample is stained with 4′,6-diamidino-2-phenylindole (DAPI) for imaging (e.g., fluorescence microscopy).

The use of any type of tissue sample in the methods disclosed herein is contemplated by the present disclosure (e.g., any of the tissue types described herein). In some embodiments, the tissue is epithelial tissue, connective tissue, muscular tissue, cardiac tissue, brain tissue, nervous tissue, gastrointestinal tract tissue, lymph node tissue, renal tissue, or tumor tissue. In certain embodiments, the intact tissue is a fixed tissue sample. In certain embodiments, the tissue sample is brain tissue for imaging neurons.

In some embodiments, the tissue sample comprises one or more cell types. Thus, the use of any type of cell in the methods disclosed herein is contemplated by the present disclosure (e.g., any of the cell types described herein). In some embodiments, the tissue sample comprises one or more cell types selected from the group consisting of stem cells, progenitor cells, neuronal cells, astrocytes, dendritic cells, endothelial cells, microglia, oligodendrocytes, muscle cells, myocardial cells, mesenchymal cells, epithelial cells, immune cells, hepatic cells, smooth and skeletal muscle cells, hematopoietic cells, lymphocytes, monocytes, neutrophils, macrophages, natural killer cells, mast cells, adipocytes, and neurons. In some embodiments, the cell is a mammalian cell. In certain embodiments, the cell is a human cell.

The present disclosure also contemplates performing the methods for profiling RNA expression and/or translation described herein on multiple tissue samples simultaneously. In some embodiments, the method is performed on multiple tissue samples of the same tissue type. In some embodiments, the method is performed on multiple cells comprising cells of different cell types. In certain embodiments, the method is performed on at least 10 cells, at least 50 cells, at least 75 cells, at least 100 cells, at least 200 cells, at least 300 cells, at least 400 cells, at least 500 cells, at least 750 cells, at least 1,000 cells, at least 2,000 cells, at least 5,000 cells, at least 7,500 cells, at least 10,000 cells, at least 20,000 cells, at least 50,000 cells, at least 75,000 cells, at least 100,000 cells, at least 125,000 cells, at least 150,000 cells, at least 175,000 cells, or at least 200,000 cells. In some embodiments, the method is performed on a test sample and a control tissue sample. In some embodiments, the method is performed on diseased and normal tissue samples. In some embodiments, the method is performed on treated and untreated tissue samples.

RNAs profiled in the methods described herein may be transcripts that have been expressed from the genomic DNA of the cell. In some embodiments, the RNAs of interest are messenger RNA (mRNA). In some embodiments, the RNAs of interest are ribosomal RNA (rRNA). In some embodiments, the RNAs of interest comprise transcripts that have not yet been processed (e.g., pre-mRNA). In some embodiments, the RNAs of interest are transfer RNAs (tRNAs). In some embodiments, the RNAs of interest are mRNAs that are actively being translated (i.e., are bound to a ribosome). The methods described herein may be used to profile expression and/or translation of one RNA in a tissue sample at a time, or of multiple RNAs simultaneously. In some embodiments, RNA expression and/or translation is profiled for more than 1, more than 2, more than 3, more than 4, more than 5, more than 10, more than 20, more than 30, more than 40, more than 50, more than 100, more than 200, more than 500, more than 1000, more than 2000, more than 3000, more than 4000, more than 5000, more than 6000, more than 7000, more than 8000, more than 9000, or more than 10,000 RNAs simultaneously. In some embodiments, the step of sequencing is repeated two, three, four, five, or more than five times to profile additional RNAs each time.

In some embodiments, the methods for profiling RNA expression and/or translation described herein may be combined with methods for profiling additional molecules within the cell. For example, methods for profiling other types of molecules (e.g., DNAs, proteins, carbohydrates, amino acids, metabolites, or lipids) may be combined with the methods described herein. In some embodiments, methods such as those described in International PCT Application Publication Nos. WO 2022/236011 (published Nov. 10, 2022), WO 2022/093940 (published May 5, 2022), WO 2023/278409 (published Jan. 5, 2023, WO 2022/178274 (published Aug. 25, 2022), WO 2022/261481 (published Dec. 15, 2022), and WO 2023/018756 (published Feb. 16, 2023) may be combined with the methods described herein.

### Methods for Diagnosing a Disease or Disorder in a Subject

In another aspect, the present disclosure provides methods for diagnosing a disease or disorder in a subject. For example, the methods for profiling RNA expression and/or translation described herein may be performed on a tissue sample taken from a subject (e.g., a subject who is thought to have or is at risk of having a disease or disorder, or a subject who is healthy or thought to be healthy). The expression and/or translation of various RNAs in the tissue sample can then be compared to the expression and/or translation of the same RNAs in a non-diseased tissue sample (e.g., a tissue from a healthy individual, or multiple cells from a population of healthy individuals). Any difference in the RNA expression and/or translation profiles of the tissue sample (including of a single RNA or of multiple RNAs of interest, e.g., a specific disease signature) relative to one or more non-diseased tissue samples may indicate that the subject has the disease or disorder.

In some embodiments, the present disclosure provides methods for diagnosing a disease or disorder in a subject, the method comprising:
a) contacting a tissue sample from a subject with 1) one or more sets of oligonucleotide probes, wherein each set of oligonucleotide probes comprises a first probe and a second probe, and 2) a third oligonucleotide probe, wherein
   i) the first probe comprises a portion that is complementary to at least a portion of the third probe, a portion that is complementary to an RNA of interest, and a portion that is complementary to a portion of the second probe;
   ii) the second probe comprises a portion that is complementary to a portion of the first probe, a portion that is complementary to the RNA of interest, and an oligonucleotide barcode sequence; and
   iii) the third probe comprises a portion that is complementary to the first probe and at least one crosslinking moiety;
b) co-polymerizing the one or more sets of probes, the third probe, and a polymeric matrix to form an oligonucleotide-polymeric matrix hybrid, wherein the first probe is hybridized to the RNA of interest;
c) removing unbound biomolecules from the oligonucleotide-polymeric matrix hybrid;
d) ligating the 5′ end and the 3′ end of the second probe together to produce a circular oligonucleotide;
e) performing rolling circle amplification to amplify the circular oligonucleotide using the first probe as a primer, thereby producing one or more concatenated amplicons;
f) embedding the one or more concatenated amplicons in the polymeric matrix; and
g) sequencing the concatenated amplicons, or a portion thereof, embedded in the polymeric matrix to determine the identity and location of each RNA of interest in the tissue sample;
wherein a difference in the profile of RNA expression and/or translation in the tissue sample from the subject relative to one or more non-diseased tissue samples indicates that the subject has the disease or disorder.

In some embodiments, RNA expression and/or translation in one or more non-diseased tissue samples is profiled simultaneously alongside the tissue taken from a subject using the methods disclosed herein as a control experiment. In some embodiments, the RNA expression and/or translation profile of one or more non-diseased tissue samples that is compared to expression and/or translation in a diseased tissue sample comprises reference data from when the method was performed on one or more non-diseased tissue samples previously. Expression and/or translation of a single RNA may be profiled in a tissue sample to diagnose a disease or disorder in a subject using the methods disclosed herein, or expression and/or translation of multiple different RNAs may be profiled in the tissue sample simultaneously. In some embodiments, the difference in the profile of RNA expression and/or translation comprises one or more mutations in an RNA (e.g., a single base substitution). In some embodiments, the difference in the profile of RNA expression and/or translation comprises increased or decreased expression and/or translation of one or more RNAs. In some embodiments, the difference in the profile of RNA expression and/or translation comprises RNAs expressed at different levels in different cell types or subcellular locations.

Diagnosis of any disease or disorder is contemplated by the methods described herein. In some embodiments, the disease or disorder is a genetic disease, a proliferative disease, an inflammatory disease, an autoimmune disease, a liver disease, a spleen disease, a renal disease, a pulmonary disease, a hematological disease, a neurological disease, a psychiatric disease, a gastrointestinal (GI) tract disease, a genitourinary disease, an infectious disease, a musculoskeletal disease, an endocrine disease, a metabolic disorder, an immune disorder, or a cardiovascular disease.

In some embodiments, the tissue sample comprises epithelial tissue, connective tissue, muscular tissue, cardiac tissue, brain tissue, nervous tissue, or tumor tissue. In some embodiments, the tissue is a tissue sample from a subject. In some embodiments, the subject is a non-human experimental animal (e.g., a mouse, a rat, a dog, a pig, a non-human primate). In some embodiments, the subject is a domesticated animal. In some embodiments, the subject is a non-human primate. In some embodiments, the subject is a human. In some embodiments, the tissue sample comprises a fixed tissue sample. In certain embodiments, the tissue sample is a biopsy (e.g., bone, bone marrow, breast, gastrointestinal tract, lung, liver, pancreas, prostate, brain, nerve, renal, endometrial, cervical, lymph node, muscle, heart, or skin biopsy). In certain embodiments, the biopsy is a tumor biopsy.

Methods of Screening for an Agent capable of Modulating RNA Expression and/or Translation

In another aspect, the present disclosure provides methods for screening for an agent (e.g., a therapeutic agent, or any kind of stimulus, such as a mechanical force, light, heat, electricity, etc.) capable of modulating RNA expression and/or translation. For example, the methods for profiling RNA expression and/or translation described herein may be performed on a tissue sample in the presence of one or more candidate agents. The expression and/or translation of various RNAs in the tissue sample (e.g., a normal tissue sample, a diseased tissue sample, etc.) can then be compared to the expression and/or translation of the same RNAs in a tissue sample that was not exposed to the one or more candidate agents. Any difference in the RNA expression and/or translation profile relative to the tissue sample that was not exposed to the candidate agent(s) may indicate that expression and/or translation of the RNAs is modulated by the candidate agent(s).

In certain embodiments, the present disclosure provides methods of screening for an agent capable of modulating expression and/or translation of one or more RNAs, the method comprising:
a) contacting a tissue sample that is being treated with or has been treated with a candidate agent with 1) one or more sets of oligonucleotide probes, wherein each set of oligonucleotide probes comprises a first probe and a second probe, and 2) a third oligonucleotide probe, wherein
   i) the first probe comprises a portion that is complementary to at least a portion of the third probe, a portion that is complementary to an RNA of interest, and a portion that is complementary to a portion of the second probe;
   ii) the second probe comprises a portion that is complementary to a portion of the first probe, a portion that is complementary to the RNA of interest, and an oligonucleotide barcode sequence; and
   iii) the third probe comprises a portion that is complementary to the first probe and at least one crosslinking moiety;
b) co-polymerizing the one or more sets of probes, the third probe, and a polymeric matrix to form an oligonucleotide-polymeric matrix hybrid, wherein the first probe is hybridized to the RNA of interest;
c) removing unbound biomolecules from the oligonucleotide-polymeric matrix hybrid;
d) ligating the 5′ end and the 3′ end of the second probe together to produce a circular oligonucleotide;
e) performing rolling circle amplification to amplify the circular oligonucleotide using the first probe as a primer, thereby producing one or more concatenated amplicons;
f) embedding the one or more concatenated amplicons in the polymeric matrix; and
g) sequencing the concatenated amplicons, or a portion thereof, embedded in the polymeric matrix to determine the identity and location of each RNA of interest in the tissue sample;
wherein a difference in the profile of RNA expression and/or translation in the tissue sample in the presence of the candidate agent relative to in the absence of the candidate agent indicates that the candidate agent modulates expression and/or translation of one or more RNAs.

In some embodiments, the difference in the profile of RNA expression and/or translation comprises one or more mutations in an RNA (e.g., a single base substitution). In some embodiments, the difference in the profile of RNA expression and/or translation comprises increased or decreased expression and/or translation of one or more RNAs. In some embodiments, the difference in the profile of RNA expression and/or translation comprises RNAs expressed and/or translated at different levels in difference cell types or subcellular locations.

In some embodiments, the candidate agent is a small molecule, a protein, a peptide, a nucleic acid, a CRISPR-based agent, a lipid, or a carbohydrate. In some embodiments, the candidate agent comprises a known drug, an FDA-approved drug, a drug candidate, or a drug undergoing clinical trials. In certain embodiments, the protein is an antibody. In certain embodiments, the protein is an antibody fragment or an antibody variant. In certain embodiments, the protein is a receptor. In certain embodiments, the protein is a cytokine. In certain embodiments, the nucleic acid is an mRNA, an antisense RNA, an miRNA, an siRNA, an RNA aptamer, a double stranded RNA (dsRNA), a short hairpin RNA (shRNA), an antisense oligonucleotide (ASO), a DNA vector, or a viral vector. In some embodiments, multiple candidate agents are provided as a screening library. Any candidate agent may be screened using the methods described herein. In particular, any candidate agents thought to be capable of modulating RNA expression and/or translation in a desired manner may be screened using the methods described herein.

In some embodiments, modulation of RNA expression and/or translation by the candidate agent is associated with reducing, relieving, or eliminating the symptoms of a disease or disorder, or preventing the development or progression of the disease or disorder. In some embodiments, the disease or disorder is a genetic disease, a proliferative disease, an inflammatory disease, an autoimmune disease, a liver disease, a spleen disease, a renal disease, a pulmonary disease, a hematological disease, a neurological disease, a psychiatric disease, a gastrointestinal (GI) tract disease, a genitourinary disease, an infectious disease, a musculoskeletal disease, an endocrine disease, a metabolic disorder, an immune disorder, or a cardiovascular disease.

### Methods for Treating a Disease or Disorder in a Subject

In another aspect, the present disclosure provides methods for treating a disease or disorder in a subject. For example, the methods for profiling RNA expression and/or translation described herein may be performed in a tissue sample taken from a subject (e.g., a subject who is thought to have or is at risk of having a disease or disorder). The profile of RNA expression and/or translation in the tissue sample can then be compared to the RNA expression and/or translation profile from reference data of a non-diseased tissue sample. A treatment for the disease or disorder may then be administered to the subject if any difference in the RNA expression and/or translation profile to a non-diseased tissue sample is observed.

In some embodiments, the present disclosure provides methods for treating a disease or disorder in a subject comprising the steps of:
a) contacting a tissue sample from a subject with 1) one or more sets of oligonucleotide probes, wherein each set of oligonucleotide probes comprises a first probe and a second probe, and 2) a third oligonucleotide probe, wherein
   i) the first probe comprises a portion that is complementary to at least a portion of the third probe, a portion that is complementary to an RNA of interest, and a portion that is complementary to a portion of the second probe;
   ii) the second probe comprises a portion that is complementary to a portion of the first probe, a portion that is complementary to the RNA of interest, and an oligonucleotide barcode sequence; and
   iii) the third probe comprises a portion that is complementary to the first probe and at least one crosslinking moiety;
b) co-polymerizing the one or more sets of probes, the third probe, and a polymeric matrix to form an oligonucleotide-polymeric matrix hybrid, wherein the first probe is hybridized to the RNA of interest;
c) removing unbound biomolecules from the oligonucleotide-polymeric matrix hybrid;
d) ligating the 5′ end and the 3′ end of the second probe together to produce a circular oligonucleotide;
e) performing rolling circle amplification to amplify the circular oligonucleotide using the first probe as a primer, thereby producing one or more concatenated amplicons;
f) embedding the one or more concatenated amplicons in the polymeric matrix;
g) sequencing the concatenated amplicons, or a portion thereof, embedded in the polymeric matrix to determine the identity and location of each RNA of interest in the tissue sample; and
h) administering a treatment for the disease or disorder to the subject if a difference in the RNA expression and/or translation profile of the tissue sample from the subject relative to one or more non-diseased tissue samples is observed.

In some embodiments, RNA expression and/or translation in one or more non-diseased tissue samples is profiled simultaneously using the methods disclosed herein as a control experiment. In some embodiments, the RNA expression and/or translation profile of one or more non-diseased tissue samples that is compared to the profile of a diseased tissue sample comprises reference data from a time the method was performed on a non-diseased tissue sample previously. In some embodiments, the difference in the profile of RNA expression and/or translation comprises one or more mutations in an RNA (e.g., a single base substitution). In some embodiments, the difference in the profile of RNA expression and/or translation comprises increased or decreased expression and/or translation of one or more RNAs. In some embodiments, the difference in the profile of RNA expression and/or translation comprises RNAs expressed and/or translated at different levels in different tissue types or subcellular locations.

Any suitable treatment for a disease or disorder may be administered to the subject. In some embodiments, the treatment comprises administering a therapeutic agent. In some embodiments, the treatment comprises administering a prophylactic agent. In some embodiments, the treatment comprises surgery. In some embodiments, the treatment comprises imaging. In some embodiments, the treatment comprises performing further diagnostic methods. In some embodiments, the treatment comprises radiation therapy. In some embodiments, the treatment comprises a change in diet or other lifestyle change. In some embodiments, the therapeutic agent is a small molecule, a protein, a peptide, a nucleic acid, a CRISPR-based agent, a lipid, a carbohydrate, or a combination thereof. In some embodiments, the therapeutic agent is a known drug and/or an FDA-approved drug. In certain embodiments, the protein is an antibody. In certain embodiments, the protein is an antibody fragment or an antibody variant. In certain embodiments, the protein is a receptor, or a fragment or variant thereof. In certain embodiments, the protein is a cytokine. In certain embodiments, the nucleic acid is an mRNA, an antisense RNA, an miRNA, an siRNA, an RNA aptamer, a double stranded RNA (dsRNA), a short hairpin RNA (shRNA), an antisense oligonucleotide (ASO), a DNA vector, or a viral vector.

Treatment of any disease or disorder is contemplated by the methods described herein. In some embodiments, the disease or disorder is a genetic disease, a proliferative disease, an inflammatory disease, an autoimmune disease, a liver disease, a spleen disease, a renal disease, a pulmonary disease, a hematological disease, a neurological disease, a psychiatric disease, a gastrointestinal (GI) tract disease, a genitourinary disease, an infectious disease, a musculoskeletal disease, an endocrine disease, a metabolic disorder, an immune disorder, or a cardiovascular disease.

In some embodiments, the subject is a human. In some embodiments, the sample comprises a biological sample. In some embodiments, the sample comprises a tissue sample. In certain embodiments, the tissue sample is a biopsy (e.g., bone, bone marrow, breast, gastrointestinal tract, lung, liver, pancreas, prostate, brain, nerve, renal, endometrial, cervical, lymph node, muscle, or skin biopsy). In certain embodiments, the biopsy is a tumor biopsy. In certain embodiments, the biopsy is a solid tumor biopsy. In certain embodiments, the RNA expression and/or translation profile of the biological sample informs prognostic decisions that guide therapies including but not limited to, pharmacological interventions for treating various conditions such as diabetes, psychiatric disorders, liver disease, kidney disease, blood disease, endocrine or exocrine disorders, heart disease, cancer therapies such as chemotherapy, targeted therapies, immunotherapy (e.g., checkpoint inhibition, CAR-T, cancer vaccines, etc.), metabolic disorders, or immune and autoimmune disorders.

### Oligonucleotide Probes

The present disclosure also provides oligonucleotide probes for use in the methods and systems for profiling RNA expression and/or translation described herein. In one aspect, the present disclosure provides a sets of oligonucleotides probes comprising: a first probe (also referred to herein as the “primer” probe) comprising a portion that is complementary to at least a portion of the third probe (also referred to herein as the “adapter” probe), a portion that is complementary to an RNA of interest, and a portion that is complementary to a portion of the second probe (also referred to herein as the “padlock” probe); a second probe comprising a portion that is complementary to a portion of the first probe, a portion that is complementary to the RNA of interest, and an oligonucleotide barcode sequence; and a third probe comprising a portion that is complementary to the first probe and at least one crosslinking moiety.

In some embodiments, the present disclosure provides sets of oligonucleotides probes further comprising a fourth probe (also referred to herein as the “splint” probe) comprising a portion complementary to the third probe, a portion complementary to at least a portion of a ribosomal RNA (rRNA) within a ribosome, and a portion complementary to the second probe.

All of the probes described herein may optionally have spacers or linkers of various nucleotide lengths in between each of the recited components, or the components of the oligonucleotide probes may be joined directly to one another (i.e., by a phosphodiester bond). All of the probes described herein may comprise standard nucleotides, or some of the standard nucleotides may be substituted for any modified nucleotides described herein or known in the art.

The present disclosure contemplates any arrangement of the portions of the first probe. In some embodiments, each first probe comprises the structure 5′-[portion complementary to portion of third probe]-[portion complementary to RNA of interest]-[portion complementary to portion of second probe]-3′, wherein each instance of]-[represents an optional linker (e.g., a nucleotide linker). In some embodiments, “]-[” represents a direct linkage between the two portions of the first probe (i.e., a phosphodiester bond). In some embodiments, the first probes described herein may comprise standard nucleotides (e.g., DNA, RNA), or some of the standard nucleotides may be substituted for any modified nucleotides known in the art. In some embodiments, portions or all of the first probes described herein may comprise modified nucleotides (e.g., LNA nucleotides) or nucleotide analogs.

In some embodiments, the portion of the first probe that is complementary to at least a portion of the third probe is 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 nucleotides in length. In certain embodiments, the portion of the first probe that is complementary to at least a portion of the third probe is about 24 nucleotides in length. In some embodiments, the portion of the first probe that is complementary to a portion of the third probe comprises the sequence CCTACCAGTACGACGTATTTAGCAA (SEQ ID NO: 2) at its 5′ end, or a sequence at least 80%, at least 85%, at least 90%, at least 95%, or at least 99% identical to the sequence CCTACCAGTACGACGTATTTAGCAA (SEQ ID NO: 2). In some embodiments, the portion of the first probe that is complementary to an RNA of interest and the portion of the first probe that is complementary to a portion of the second probe together comprise 40, 41, 42, 43, 44, 45, 46, 47, 48, 49, 50, 51, 52, 53, 54, 55, 56, 57, 58, 59, 60, 61, 62, 63, 64, 65, 66, 67, 68, 69 70, 71, 72, 73, 74, 75, 76, 77, 78, 79, or 80 nucleotides in length. In some embodiments, the portion of the first probe that is complementary to an RNA of interest and the portion of the first probe that is complementary to a portion of the second probe together comprise about 58 nucleotides in length.

Example first probe sequences for profiling particular genes using the methods provided herein are as follows:

| Gene | Probe Sequence | SEQ ID NO |
|---|---|---|
| a2m | CCTACCAGTACGACGTATTTAGCAAGGCTT | 5 |
| | CCCATAGGTGTATATGCGTGGATTAGAAA | |
| a2m | CCTACCAGTACGACGTATTTAGCAAGCCG | 6 |
| | GAAGACCGATATTTGGGTGGATTAGAAA | |
| abcc9 | CCTACCAGTACGACGTATTTAGCAACCCGG | 7 |
| | GTTTGATGTAAGCCGTGGATTGGAAA | |
| abcc9 | CCTACCAGTACGACGTATTTAGCAAGGTCA | 8 |
| | TCTCACCCATGGATAGGGTGGATTGGAAA | |
| abi3bp | CCTACCAGTACGACGTATTTAGCAACCTGG | 9 |
| | TTTTCTTGTGGCATGTGAGTGGATTCGAAA | |
| abi3bp | CCTACCAGTACGACGTATTTAGCAACCTGC | 10 |
| | AGAAATTGGCTCACGTGGATTCGAAA | |
| acbd7 | CCTACCAGTACGACGTATTTAGCAAATCAG | 11 |
| | CTCTCTCGCTTTAGAGTGGATTTGAAA | |
| acbd7 | CCTACCAGTACGACGTATTTAGCAATCCCA | 12 |
| | TTTGGCCTTACCCTGTGGATTTGAAA | |

The present disclosure contemplates any arrangement of the portions of the second probe. In some embodiments, the second probe in the set of probes comprises the structure 5′-[portion complementary to portion of first probe]-[portion complementary to RNA of interest]-[oligonucleotide barcode sequence]-[portion complementary to portion of first probe]-3′, wherein each instance of]-[represents an optional linker (e.g., a nucleotide linker). In some embodiments, “]-[” represents a direct linkage between the two portions of the second probe (i.e., a phosphodiester bond). In some embodiments, the second probes described herein may comprise standard nucleotides (e.g., DNA, RNA), or some of the standard nucleotides may be substituted for any modified nucleotides known in the art. In some embodiments, portions of the second probes described herein may comprise modified nucleotides (e.g., LNA nucleotides) or nucleotide analogs.

In some embodiments, the second probe includes an oligonucleotide barcode sequence made up of a specific sequence of nucleotides. In some embodiments, the oligonucleotide barcode sequence is 5, 6, 7, 8, 9, 10, 11, 12, 13, 14, or 15 nucleotides in length. In some embodiments, the barcodes of the second probe may comprise gene-specific sequences used to identify RNAs of interest during sequencing as described further below (i.e., each barcode sequence is associated with a specific gene or transcript). The use of the barcodes on probes analogous to those described herein is further described in, for example, International Patent Application Publication No. WO 2019/199579, published Oct. 17, 2019, and Wang et al., Science 2018, 361, 380, each of which is incorporated herein by reference.

Example second probe sequences for profiling particular genes using the methods provided herein are as follows:

| | | SEQ |
|---|---|---|
| Gene | Probe Sequence | ID NO |
| a2m | /5Phos/ATCCACCACACGGACACATTCATCTCT | 13 |
| | AATTACTTTTCCATACACTAAAGTTTCTA | |
| a2m | /5Phos/ATCCACGGAAGACCAAATCCACCTTG | 14 |
| | TAATTACTTTTCCATACACTAAAGTTTCTA | |
| abcc9 | /5Phos/ATCCACTGACATGTTTCAGAACGGGC | 15 |
| | AATTACAAAAGCATACACTAAAGTTTCCA | |
| abcc9 | /5Phos/ATCCACAGAAGTCGAGAGCCGAAGGA | 16 |
| | TAATTACAAAAGCATACACTAAAGTTTCCA | |
| abi3bp | /5Phos/ATCCACCCACTGACGCATTTCTCCCAG | 17 |
| | AATTACAAAACCATACACTAAAGTTTCGA | |
| abi3bp | /5Phos/ATCCACCACTCTTGGGTCAGCTGATTA | 18 |
| | ATTACAAAACCATACACTAAAGTTTCGA | |
| acbd7 | /5Phos/ATCCACTATAGGCACACATGGCATCT | 19 |
| | AATTACAAAATCATACACTAAAGTTTCAA | |
| acbd7 | /5Phos/ATCCACAGATCTAACATTGCTGGGCA | 20 |
| | TGAATTACAAAATCATACACTAAAGTTTCAA | |

The present disclosure contemplates any arrangement of the portions of the third probe. In some embodiments, the third probe in the set of probes comprises the structure: 5′-[first crosslinking moiety]-[portion complementary to first probe]-3′. In certain embodiments, the third probe comprises the structure: 5′-[first crosslinking moiety]GCTA [nucleoside analog comprising second crosslinking moietyJATACGTCGTACTGGTAGG [Inverted-dT]-3′ (SEQ ID NO: 3). In certain embodiments, the third probe comprises the structure: 5′-[Acrydite]GCTA [CNVK3]ATACGTCGTACTGGTAGG [Inverted-dT]-3′ (SEQ ID NO: 4). Each instance of]-[in the third probe independently represents an optional linker (e.g., a nucleotide linker). In some embodiments,]-[represents a direct linkage between the two portions of the third probe (i.e., a phosphodiester bond). In some embodiments, the third probes described herein may comprise standard nucleotides (e.g., DNA, RNA), or some of the standard nucleotides may be substituted for any modified nucleotides known in the art. In some embodiments, portions of the third probes described herein may comprise modified nucleotides (e.g., LNA nucleotides) or nucleotide analogs. In some embodiments, the third probe is 15, 16, 17, 18, 19, 20, 21, 22, 23, 24, 25, 26, 27, 28, 29, or 30 nucleotides in length. In certain embodiments, the third probe is about 24 nucleotides in length.

In certain embodiments, the portion of the third probe that is complementary to the first probe comprises one or more nucleoside analogs comprising a second crosslinking moiety (in addition to the crosslinking moiety at the 5′ end of the probe). The second crosslinking moiety may be useful, for example, for stabilizing the complex formed between the first and third probes via the formation of a covalent bond. Any nucleoside analogs comprising a crosslinking moiety may be used in the third probe, and the present disclosure also contemplate the use of crosslinking mechanisms, such as, but not limited to, the use of cyano-modified oligonucleotides, furan-modified oligonucleotides, and psoralen. The present disclosure further recognizes crosslinks can be formed by covalent cyclobutene pyrimidine dimer (CPD) bonds induced via ultraviolet irradiation, the use of reductive amination reactions between an abasic site and the non-canonical nucleobase 2-aminopurine, and the use of oxidation reactions between a singlet oxygen and a modified nucleotide (e.g., an analogue of thymidine). In some embodiments, the nucleoside analog comprises a cyano moiety. Nucleoside analogs comprising cyano moieties useful for forming nucleic acid crosslinks are described, for example, in Yoshimura and Fujimoto, Org. Lett. 2008, 10 (15), 3227-3230, which is incorporated herein by reference. In some embodiments, the nucleoside analog comprises a furan moiety (which may, for example, be oxidized to form a 4-oxo-enal derivative, facilitating formation of a crosslink between two nucleic acids as described in Stevens et al., Nucleic Acids Research 2009, 37 (5), 1555-1565, which is incorporated herein by reference). In some embodiments, proximal thymidines on the first and the third probes are used to form a crosslink, for example, by exposing the thymidines to UV irradiation. Further methods of crosslinking two nucleic acid molecules are described, for example, in Hong and Greenberg, J. Am. Chem. Soc. 2005, 127 (30), 10510-10511 and Nejad et al., ACS Chem. Biol. 2019, 14 (7), 1481-1489, each of which is incorporated herein by reference. In some embodiments, the nucleoside analog comprises a thiol group. In some embodiments, the nucleoside analog is 3-cyanovinylcarbazole (CNVK3), Psoralen C2 phosphoramidite, 6-thio-dG, 4-thio-dT, or 4-thio-dU. In certain embodiments, the nucleotide analog is 3-cyanovinylcarbazole (CNVK3). In some embodiments, the CNVK3-containing third probe is photo crosslinked to a pyrimidine base of the first probe upon ultraviolet (UV) irradiation (e.g., at a wavelength of about 360-370 nm, for example, about 366 nm). In some embodiments, the reaction time is about 5, about 10, about 15, about 20, about 25, about 30, or more than 30 minutes. In certain embodiments, the reaction time is about 10 minutes. In some embodiments, the ratio of the third probe to the first probe added to a tissue sample is at least 3:1, at least 4:1, at least 5:1, at least 6:1, at least 7:1, at least 8:1, at least 9:1, or at least 10:1 during the crosslinking reaction. In certain embodiments, the ratio of the third probe to the first probe is about 5:1 during the crosslinking reaction.

In some embodiments, at least one crosslinking moiety of the third probe comprises acrydite. The use of other comparable mechanisms for further functionalizing the third probe is also contemplated by the present disclosure. For example, in addition to incorporating a 5′ acrylamide modification, amine-modified nucleoside analogs can be introduced into the design of the third probe. The amine moieties can, e.g., be treated with acrylic acid N-hydroxysuccinimide esters, further functionalizing the adapter with additional acrylate groups that can facilitate crosslinking.

In some embodiments, third probe comprises an inverted nucleotide at its 3′ end. In certain embodiments, the inverted nucleotide is an inverted thymine, adenine, guanine, uracil, or cytidine residue. In certain embodiments, the inverted nucleotide is an inverted thymine residue. The inverted nucleotide may help ensure that the third probe is not used as a primer during rolling circle amplification.

Example third probe sequences for use in the methods provided herein are as follows:

| | SEQ |
|---|---|
| Probe Sequence | ID NO |
| CCTACCAGTACGACGTATTTAGCAAACAAAATAGAACCGCGGT | 21 |
| CCTATTCAAAAAAAAAAAAAAAAAAAAAAAAAAAAAAAAAA | |
| AAAAAAAAAAAAAAAATATCTTTAGT*G*T*/3InvdT/ | |
| CCTACCAGTACGACGTATTTAGCAACATCGTTTATGGTCGGAA | 22 |
| CTACGACAAAAAAAAAAAAAAAAAAAAAAAAAAAAAAAAAA | |
| AAAAAAAAAAAAAAAATATCTTTAGT*G*T*/3InvdT/ | |
| CCTACCAGTACGACGTATTTAGCAAAGGTTTCCCGTGTTGAGTC | 23 |
| AAATTAAAAAAAAAAAAAAAAAAAAAAAAAAAAAAAAAAAA | |
| AAAAAAAAAAAAAATATCTTTAGT*G*T*/3InvdT/ | |
| CCTACCAGTACGACGTATTTAGCAATGTTATTGCTCAATCTCGG | 24 |
| GTGGCTAAAAAAAAAAAAAAAAAAAAAAAAAAAAAAAAAAA | |
| AAAAAAAAAAAAAAATATCTTTAGT*G*T*/3InvdT/ | |
| CCTACCAGTACGACGTATTTAGCAAAGATAGTCAAGTTCGACC | 25 |
| GTCTTCTAAAAAAAAAAAAAAAAAAAAAAAAAAAAAAAAAAA | |
| AAAAAAAAAAAAAAATATCTTTAGT*G*T*/3InvdT/ | |

The present disclosure contemplates any arrangement of the portions of the fourth probe. In some embodiments, the set of probes comprises a fourth probe for profiling expression of RNAs that are actively being translated in a tissue sample. The fourth probe in the set of probes comprises a portion complementary to the third probe, a portion complementary to at least a portion of a ribosomal RNA (rRNA), and a portion complementary to the second probe. In some embodiments, the portion of the fourth probe that is complementary to a portion of the third probe comprises the sequence CCTACCAGTACGACGTATTTAGCAA (SEQ ID NO: 2) at its 5′ end, or a sequence at least 80%, at least 85%, at least 90%, at least 95%, or at least 99% identical to the sequence CCTACCAGTACGACGTATTTAGCAA (SEQ ID NO: 2). In some embodiments, the portion of the fourth probe that is complementary to at least a portion of the rRNA is complementary to at least a portion of an 18S rRNA. In some embodiments, the fourth probe comprises the structure 5′-[portion complementary to third probe]-[portion complementary to rRNA]-[portion complementary to portion of second probe]-3′, wherein “]-[” represents an optional linker (e.g., a nucleotide linker). In some embodiments, “]-[” represents a direct linkage between the two portions of the primer probe (i.e., a phosphodiester bond). In some embodiments, the fourth probes described herein may comprise standard nucleotides (e.g., DNA, RNA), or some of the standard nucleotides may be substituted for any modified nucleotides known in the art. In some embodiments, portions of the fourth probes described herein may comprise modified nucleotides (e.g., LNA nucleotides) or nucleotide analogs. In some embodiments, the portion of the fourth probe that is complementary to at least a portion of an rRNA is 5, 6, 7, 8, 9, 10, 11, 12, 13, 14, 15, 16, 17, 18, 19, 20, 21, 22, 23, 24, 25, 26, 27, 28, 29, or 30 nucleotides in length.

In some embodiments, the portion of the fourth probe that is complementary to the third probe comprises the same sequence as the portion of the first probe that is complementary to the third probe. In certain embodiments, the second crosslinking moiety of the third probe is covalently crosslinked to a pyrimidine base of the fourth probe by photo crosslinking.

In embodiments comprising the use of a fourth probe for profiling RNAs actively being translated by a ribosome, the second probe may comprise the structure 5′-[portion complementary to portion of fourth probe]-[portion complementary to portion of first probe]-[portion complementary to RNA of interest]-[oligonucleotide barcode sequence]-[portion complementary to portion of fourth probe]-3′.

Example fourth probe sequences for profiling particular genes using the methods provided herein are as follows:

| Gene | Probe Sequence | SEQ ID NO |
|---|---|---|
| a2m | /5Phos/AAGATAAATTTCTAATCCTACACACGGACACA | 26 |
| | TTCATCTCTATTACTTTTCCATACACTA | |
| a2m | /5Phos/AAGATAAATTTCTAATCCTAGGAAGACCAAAT | 27 |
| | CCACCTTGTATTACTTTTCCATACACTA | |
| abcc9 | /5Phos/AAGATAAATTTCCAATCCTATGACATGTTTCA | 28 |
| | GAACGGGCATTACAAAAGCATACACTA | |
| abcc9 | /5Phos/AAGATAAATTTCCAATCCTAAGAAGTCGAGA | 29 |
| | GCCGAAGGATATTACAAAAGCATACACTA | |
| abi3bp | /5Phos/ATCCACCCACTGACGCATTTCTCCCAGAATTA | 30 |
| | CAAAACCATACACTAAAGTTTCGA | |
| abi3bp | /5Phos/ATCCACCACTCTTGGGTCAGCTGATTAATTAC | 31 |
| | AAAACCATACACTAAAGTTTCGA | |
| acbd7 | /5Phos/AAGATAAATTTCAAATCCTATATAGGCACACA | 32 |
| | TGGCATCTATTACAAAATCATACACTA | |
| acbd7 | /5Phos/AAGATAAATTTCAAATCCTAAGATCTAACATT | 33 |
| | GCTGGGCATGATTACAAAATCATACACTA | |

In some embodiments, the present disclosure provides a plurality of probes comprising multiple sets of oligonucleotide probes as described herein. In certain embodiments, each set of probes in the plurality comprises a primer probe that is complementary to a different RNA in a tissue sample. In some embodiments, the plurality of probes comprises more than 1, more than 2, more than 3, more than 4, more than 5, more than 10, more than 20, more than 30, more than 40, more than 50, more than 100, more than 200, more than 500, more than 1000, more than 2000, more than 3000, more than 4000, more than 5000, more than 6000, more than 7000, more than 8000, more than 9000, or more than 10,000 sets of probes.

### Kits

Also provided by the disclosure are kits. In one aspect, the kits provided may comprise one or more of the probes described herein. In some embodiments, the kits comprise any of the sets of probes or pluralities of probes described herein. In certain embodiments, the kits comprise more than 1, more than 2, more than 3, more than 4, more than 5, more than 10, more than 20, more than 30, more than 40, more than 50, more than 100, more than 200, more than 500, more than 1000, more than 2000, more than 3000, more than 4000, more than 5000, more than 6000, more than 7000, more than 8000, more than 9000, or more than 10,000 sets of probes or pluralities of probes. In some embodiments, the kits may further comprise a container (e.g., a vial, ampule, bottle, and/or dispenser package, or other suitable container). The kits may also comprise tissue samples for performing control experiments. In some embodiments, the kits may further comprise other reagents for performing the methods disclosed herein (e.g., enzymes, such as a ligase, a polymerase (e.g., a DNA polymerase), and/or an RNase, nucleotides comprising a nucleophile (e.g., amine-modified nucleotides) as described herein, buffers, and/or reagents and monomers for making a polymeric matrix (e.g., a polyacrylamide matrix)).

In some embodiments, the kits are useful for profiling RNA expression and/or translation in a tissue sample. In some embodiments, the kits are useful for diagnosing a disease in a subject. In some embodiments, the kits are useful for screening for an agent capable of modulating expression and/or translation of one or more RNAs. In some embodiments, the kits are useful for diagnosing a disease or disorder in a subject. In some embodiments, the kits are useful for treating a disease or disorder in a subject. In certain embodiments, a kit described herein further includes instructions for using the kit.

### Samples

In one aspect, the present disclosure provides a sample for profiling RNA expression and/or translation in a tissue sample. In some embodiments, such a sample comprises one or more concatenated amplicons or polymeric matrix-embedded concatenated amplicons produced by any of the methods described herein.

In some embodiments, a sample comprises a concatenated amplicon produced using the methods described herein. In some embodiments, the concatenated amplicons are produced using rolling circle amplification (RCA). In some embodiments, the concatenated amplicons comprise one or more nucleotide analogs modified with reactive chemical groups (e.g., amine modified nucleotides or any nucleotides comprising a nucleophile, such as 5-(3-aminoallyl)-dUTP). Examples of other amine-modified nucleotides include, but are not limited to, a 5-Aminoallyl-dUTP moiety modification, a 5-Propargylamino-dCTP moiety modification, a N6-6-Aminohexyl-dATP moiety modification, or a 7-Deaza-7-Propargylamino-dATP moiety modification.

In some embodiments, the sample comprises concatenated amplicons embedded in a polymeric matrix as described herein. For example, the amine-modified nucleotides of the one or more concatenated amplicons may be reacted with a crosslinking agent (e.g., BS(PEG)₉) and co-polymerized with the polymeric matrix in order to embed them therein. In some embodiments, the sample further comprises one or more of the probes described herein (e.g., the first probe, second probe, third probe, and/or fourth probe).

The use of various polymeric matrices is contemplated by the present disclosure, and the samples may comprise any polymeric matrix in which the one or more concatenated amplicons can be embedded. In some embodiments, the polymeric matrix is a hydrogel. In some embodiments, the hydrogel is a polyvinyl alcohol hydrogel, a polyethylene glycol hydrogel, a polyacrylate hydrogel, or a polyacrylamide hydrogel. In certain embodiments, the polymeric matrix comprises about 10-20% (e.g., about 15%) TBE-Urea. Such a hydrogel may be prepared, for example, by incubating the sample in a buffer comprising acrylamide and bis-acrylamide, removing the buffer, and incubating the sample in a polymerization mixture (comprising, e.g., ammonium persulfate and tetramethylethylenediamine).

### Systems

In one aspect, the present disclosure provides systems for profiling RNA expression and/or translation in a cell. In some embodiments, such a system comprises:
a) a tissue sample; and
b) one or more sets of oligonucleotide probes, wherein each set of probes comprises a first probe and a second probe, and a third oligonucleotide probe, wherein:
   i) the first probe comprises a portion that is complementary to at least a portion of the third probe, a portion that is complementary to an RNA of interest, and a portion that is complementary to a portion of the second probe;
   ii) the second probe comprises a portion that is complementary to a portion of the first probe, a portion that is complementary to the RNA of interest, and an oligonucleotide barcode sequence; and
   iii) the third probe comprises a portion that is complementary to the first probe and at least one crosslinking moiety.

In some embodiments, the system comprising a set of probes further comprises a fourth probe comprising a portion complementary to the third probe, a portion complementary to at least a portion of a ribosomal RNA (rRNA) within a ribosome, and a portion complementary to the second probe.

Any of the probes (i.e., the sets of probes and pluralities of probes) described herein may be used in the systems of the present disclosure. In some embodiments, a system further comprises a microscope. In some embodiments, a system further comprises a camera. In some embodiments, a system further comprises a computer. In certain embodiments, a system further comprises software running on the computer (e.g., software for viewing or processing the images observed on or captured with a microscope). In certain embodiments, a system further comprises a liquid handling system. In some embodiments, the systems further comprise one or more enzymes. In certain embodiments, the systems further comprise an RNase. In certain embodiments, the systems further comprise a DNA ligase. In certain embodiments, the systems further comprise a DNA polymerase. In some embodiments, the systems further comprise additional reagents (e.g., dyes, stains, antibodies, etc.). In certain embodiments, the systems further comprise nucleotides comprising a nucleophile (e.g., amine-modified nucleotides). In certain embodiments, the systems further comprise one or more buffers. In certain embodiments, the systems further comprise reagents and monomers for preparing a polymeric matrix (e.g., a polyacrylamide matrix)).

### EXAMPLES

### Example 1: Spatially Resolved Single-cell Transcriptional and Translatomic States in Thick Tissue Blocks

Profiling information on transcriptional and translational regulation in three-dimensional (3D) tissue microenvironments is crucial for understanding functional heterogeneity in biological tissues. Most existing in situ sequencing techniques are limited to thin tissue sections, hindering the analysis of 3D associations among cell types. The present disclosure describes the development of thick-STARmap and thick-RIBOmap, which enable high-content 3D in situ quantification of hundreds of gene transcripts and their translation in intact thick tissue samples. These techniques facilitate enzyme diffusional access by embedding probes in a hydrogel matrix and digesting the tissue before performing enzyme reactions. The methods offer significant advancements in studying complex 3D tissue microenvironments at the transcriptome and translatome levels at cellular resolution.

### Design and Validation of Thick-STARmap

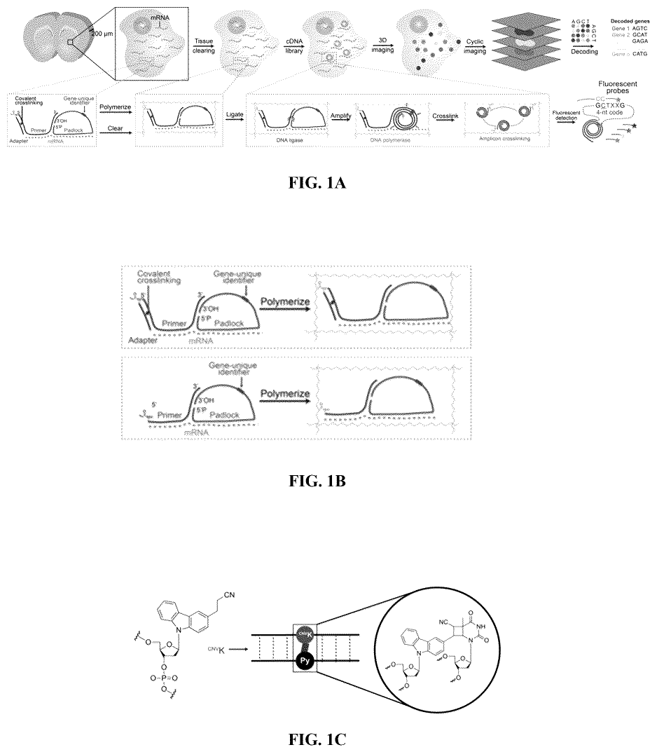

The development and implementation of in situ sequencing techniques in thick tissue blocks for 3D analysis is crucial for preserving 3D relationships among cell types within tissue structures. However, STARmap and RIBOmap rely on enzymatic reactions to generate cDNA amplicons, and tissue thickness presents a significant challenge for enzyme diffusion access. Thick-STARmap initiates by labeling cellular RNAs using a pair of primer probes (i.e., first probe) and padlock probes (i.e., second probes). Subsequently, probes and tissue are embedded in a hydrogel matrix, followed by protein digestion and lipid removal to enhance enzyme penetration, ensuring sufficient depth in thick tissue samples. In situ cDNA amplicons are then synthesized via enzymatic ligation and rolling circle amplification (RCA). Each cDNA amplicon contains a pre-designed gene-specific identifier, which is read out through cyclic imaging (FIG. 1A).

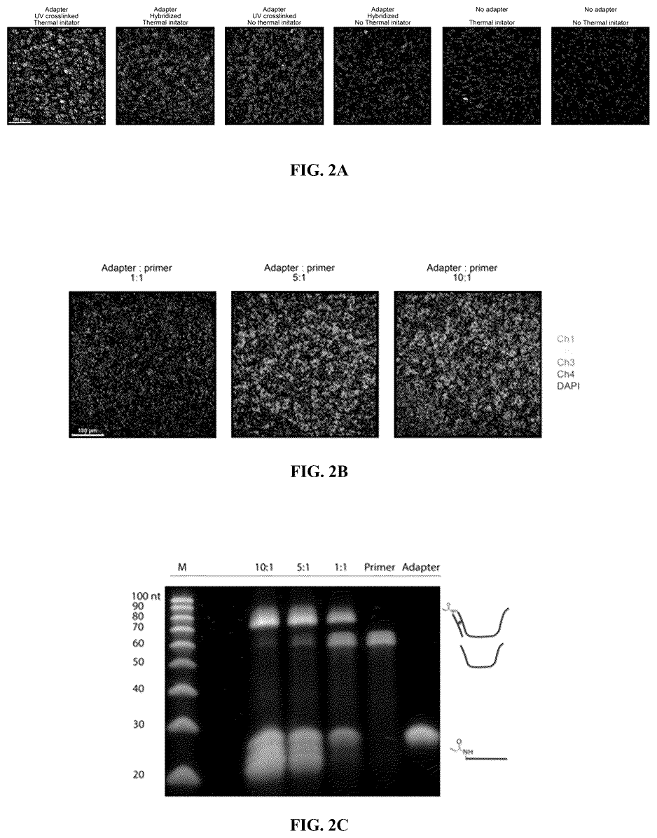

To incorporate probes into the polyacrylamide hydrogel, each primer (first probe) comprises a “flanking linker sequence” at the 5′ end of the RNA target sequence, allowing an acrydite-modified oligonucleotide of the adapter probe (third probe) to hybridize with this flanking linker. Consequently, the probe set can be incorporated into polyacrylamide gels during polymerization. It is noteworthy that previous methods involving the direct incorporation of acrydite modifications during primer synthesis have been successful. However, such an approach is not scalable due to limitations in modified oligonucleotide synthesis (FIG. 1B). Importantly, the approach described herein enables high multiplexing capacity due to the utilization of the same flanking linker sequence and corresponding adapter sequence (i.e., corresponding sequence of the third probe) for all primer probes (first probes), enabling simultaneous measurement of hundreds of transcripts and providing a more versatile and scalable solution for transcript detection. Additionally, to stabilize the adapter-primer complex (i.e., complex between the third and first probes) via covalent bond formation, a nucleoside analog, 3-cyanovinylcarbazole nucleoside (^{CNV}K)³, is incorporated into the adapter probe (third probe). The ^{CNV}K-containing adapter then undergoes rapid photo cross-linking to the complementary strand through an adjacent pyrimidine base upon 366 nm UV irradiation (FIG. 1C), which has been demonstrated to cause no damage to normal DNA^{4,5}. These findings demonstrate that the utilization of UV crosslinking results in a higher preservation rate of amplicons (FIG. 2A). It has also been demonstrated that an adapter-primer ratio (i.e., ratio of the third probe to the first probe) of 5:1 is sufficient, and increasing this ratio does not yield a higher number of amplicons. Finally, it has also been shown that an adapter-primer ratio of 5:1 and a reaction time of 10 minutes result in nearly 100% crosslinking yield.

Ammonium persulfate (APS)-initiated polymerization is a rapid process, which can result in heterogeneous gel formation as the gel solidifies before APS can adequately penetrate the central region. To address this issue, VA-044, a thermal initiator that operates at 40 °C, was incorporated as an additional initiator, which allows for a more controlled polymerization rate. This approach promotes a more uniform polymerization process throughout the gel, enhancing the crosslinking of probes into the hydrogel. Then, enzymatic ligation and RCA were carried out to construct the in situ cDNA amplicons for a longer time. Aminoallyl-dUTP was spiked into the RCA reaction, and the primary amines were then cross-linked to the polymeric matrix using BS(PEG)₉, for high-quality sequencing reads.

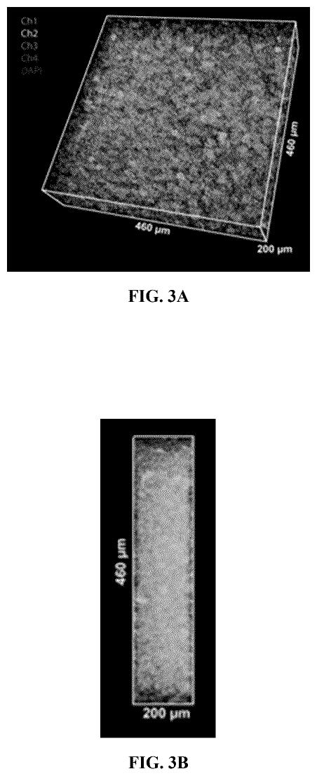

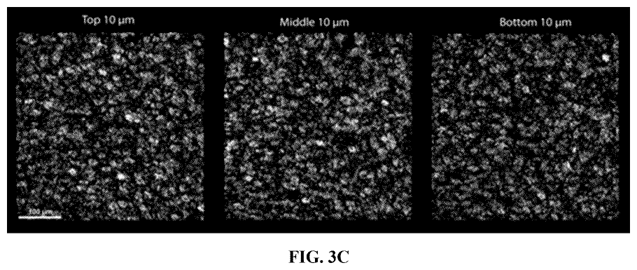

Overall, the ability to perform single-cell transcriptional mapping within a 200 μm tissue slice without compromising the quality of the results was demonstrated (FIG. 3). These findings indicate that amplicon density is homogeneous throughout the entire 3D tissue.

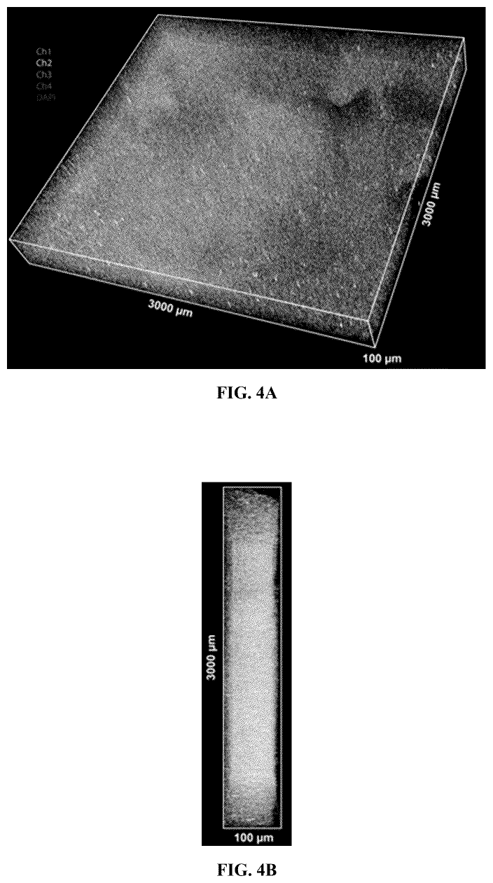

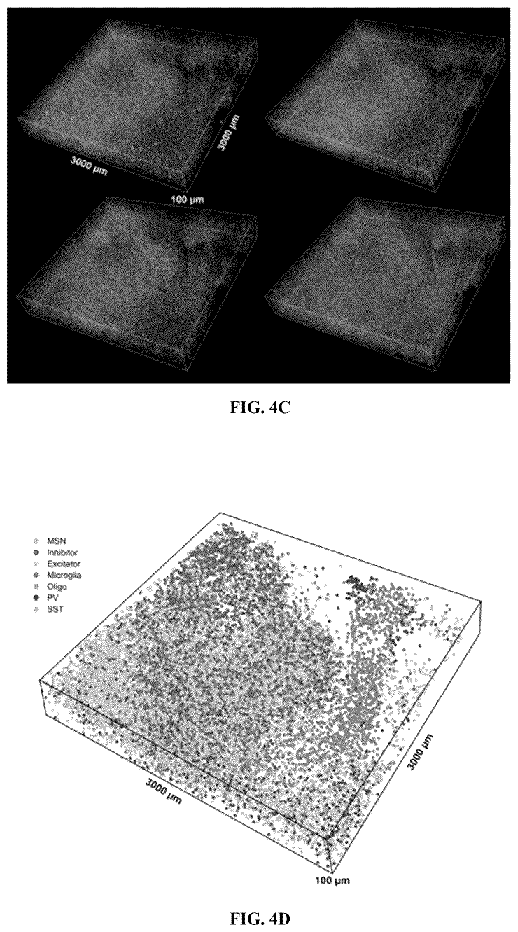

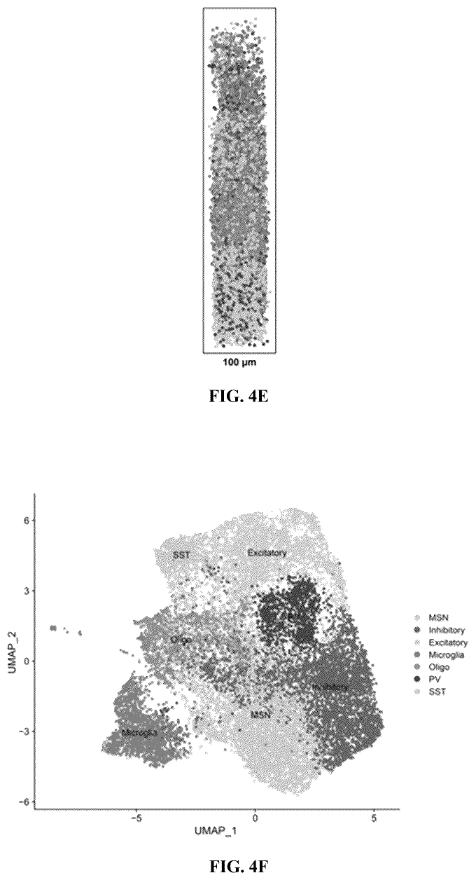

To evaluate the capability of thick-STARmap in delivering high-content 3D intact-tissue sequencing of single-cell transcriptional states with the required sensitivity and accuracy, the technique was applied to map the nucleus accumbens (NAc) region of the brain. A brain slice was transformed into a hydrogel-tissue hybrid for cyclic imaging of a selected panel of transcripts, including the 60 most differentially expressed cell-type marker genes derived from previously published scRNA-seq data⁶⁻⁹. Four cycles of fluorescence sequencing were performed to profile gene expression at single-cell resolution (FIGS. 4A-4C). Fluorescence images of cDNA amplicon dots in three sequencing rounds were registered with a phase correlation algorithm followed by local distortion registration. Subsequently, 3D cell segmentation was conducted, and RNA reads were assigned to cells. Leiden clustering revealed several major cell populations with unique gene expression and spatial patterns, including MSN neurons, excitatory neurons, oligodendrocytes, microglia, and SST neurons (FIGS. 4D-4F). These findings are in agreement with previous reports.

### Design and Validation of Thick-RIBOmap

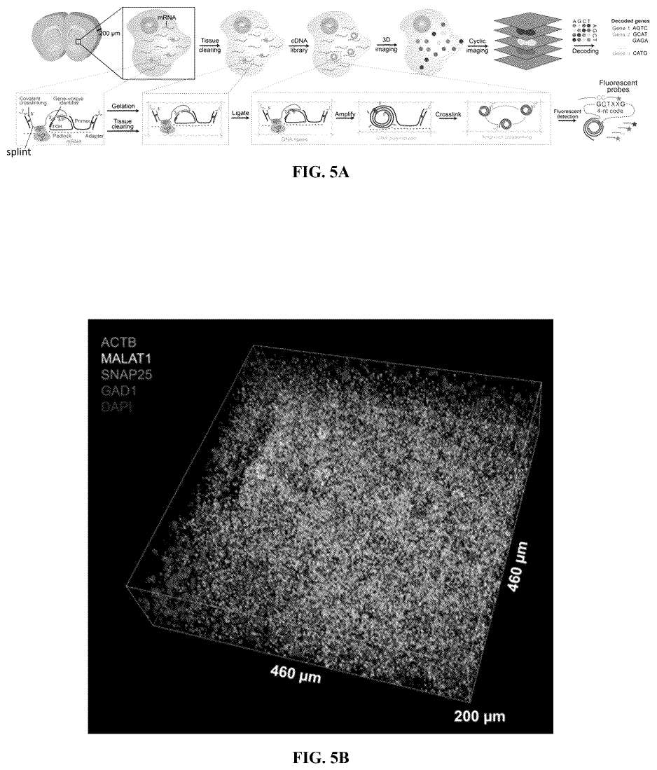

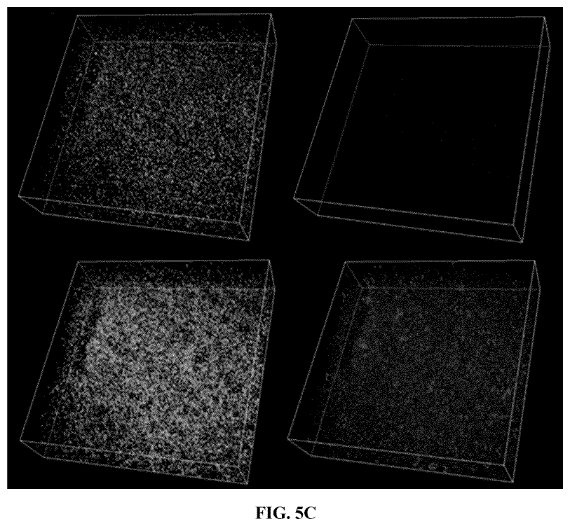

The insights gained from developing thick-STARmap were subsequently applied to establish thick-RIBOmap for the investigation of spatial translational control in thick tissue samples. In contrast to the two-probe set with an adapter probe in thick-STARmap, thick-RIBOmap employs a distinctive tri-probe design that selectively detects and amplifies ribosome-bound mRNAs. In addition to the padlock probe (second probe), the flank linker-containing primer probe (first probe), and the adapter probe (third probe), the thick-RIBOmap tri-probe set features a splint probe (i.e., the fourth probe) that hybridizes to ribosomal RNAs (rRNAs) and acts as the splint for circularizing proximal padlock probes. The splint probe also possesses the same “flanking linker sequence” at the 5′ end of the rRNA target sequence, as that of the primer probe (first probe). As such, the splint probe (fourth probe) can complex with an adapter probe (third probe) in the same manner as would a primer probe (first probe). Subsequent steps, including polymerization, at least an optional step of tissue clearing, enzymatic ligation, RCA, BS(PEG) 9 crosslinking, and barcode sequencing, are performed in the same manner as in thick-STARmap (FIG. 5A).

Although previous studies have demonstrated that RIBOmap tri-probe amplification selectively targets ribosome-bound mRNAs, this specificity was revalidated in thick tissue samples. Three protein-coding transcripts (ACTB, SNAP25, and GADI RNA) and one negative control non-coding RNA (nuclear-localized MALATI RNA) were chosen for this purpose. As anticipated, thick-RIBOmap exclusively detected cytoplasmic ACTB, SNAP25, and GADI mRNAs, while the nuclear-localized MALATI RNA was undetected, revalidating thick-RIBOmap specificity for ribosome-bound mRNAs in thick tissue contexts (FIGS. 5B-5C).

### Example 2: Materials and Methods

### Plate Coating

1. The 12-well plates were cleaned with oxygen plasma for 5 minutes (100 W, 40% O₂).
2. Prepare bind silane coating solution.
3. Add 800 μL bind silane coating solution to each well for 1-1.5 hours at room temperature.
4. Rinse plate with 500 μL ethanol three times.
5. Wells were treated with 500 μL 0.1 mg/mL Poly-D-lysine in water for 1 hour at room temperature, rinsed with water three times, and then the plates are air-dried for 1 hour.

| | | Stock | | | |
|---|---|---|---|---|---|
| Buffer | Ingredients | Conc. | Final Conc. | Amount | |
| Bind silane | Bind silane | 100% | 1% | 400 | μL |
| coating solution | Acetic acid | 100% | 10% | 4 | mL |
| | ethanol | 100% | 90% | 35.2 | mL |
| | H₂O | | | 400 | μL |
| | Total | | | 40 | mL |

### Tissue Sample Preparation and Fixation

All animal procedures adhered to the care guidelines approved by the Institutional Animal Care and Use Committee (IACUC) of the Broad Institute of MIT and Harvard, under animal protocol #0255 Aug. 19. For the experiments, C57/BL6 mice aged between 6 to 10 weeks were procured from The Jackson Laboratory (JAX).
1. Mice were anesthetized using isoflurane.
2. A transcardial perfusion was then performed: first with 50 mL of cold PBS followed by 50 mL of a 4% PFA solution.
3. The entire brain was post-fixed in 4% PFA at 4 °C for 3 hours.
4. The brain was embedded in O.C.T and frozen in liquid nitrogen.
5. Subsequent tissue sections were transferred to a cryostat and cut as 100 or 200 μm slices.
6. The tissue slices were mounted in the pretreated glass-bottom 12-well plate.
7. The brain slices were fixed with 4% PFA buffer at room temperature for 30 minutes, and again for 30 minutes at 4 °C.
8. The tissue slices were then permeabilized with 1 mL −20 °C methanol and incubated at −20 °C for one hour.

| Buffer | Ingredients | Stock Conc. | Final Conc. | Amount | |
|---|---|---|---|---|---|
| 4% PFA buffer | PFA | 16% | 4% | 2.5 | mL |
| (prepare fresh) | PBS | 10× | 1× | 1 | mL |
| | H₂O | | | 6.5 | mL |
| | Total | | | 10 | mL |

### Thick-STARmap and Thick-RIBOmap Probe Design

The design of the thick-STARmap padlock and primer probes was based on processes elucidated in Wang et al., with certain alterations: (1) For genes that have multiple transcript variants, only the shortest transcript forms were focused on. Further, only coding domains were targeted, except in the case of non-coding RNAs. (2) Probe pair hybridization sequences, constrained to a length between 40 and 46 nucleotides, were conceptualized using the Picky 2.2 software. (3) Corresponding DNA (cDNA) sequences, within the 40-46 nucleotide range, were bifurcated into two segments, each 19-25 nucleotides long, separated by an interval of 0-2 nucleotides. An equilibrium in melting temperature (Tm) was maintained between the two segments. (4) To derive the final target sequence pool for the species, the Dedupe tool from the BBmap suite was employed, filtering out overlapping and contained sequences. Additionally, to facilitate the hybridization of a primer probe with an acrydite-modified oligonucleotide (also referred to as the “adapter probe, or “third” probe), each primer probe incorporated a “flanking linker sequence” (CCTACCAGTACGACGTATTTAGCAA) (SEQ ID NO: 2) at its 5′ end.

The design of the thick-RIBOmap padlock and primer probes was based on processes described in Zeng et al. with certain alterations. In addition to the primer and padlock sequence, thick-RIBOmap requires an auxiliary splint probe. This RIBOmap splint probe is delineated into three main segments: an initial 25 nucleotide sequence at the 5′ end that corresponds with 18S ribosomal RNA (rRNA), an intermediary 50 nucleotide deoxyadenosine nucleotides (dA) strand, and a terminal 12 nucleotide padlock template on the 3′ side. A 3′ Inverted dT modification obstructs the 3′ terminus of the splint probes, preventing their use as an RCA primer. Additionally, to facilitate the hybridization of the splint probe with an acrydite-modified oligonucleotide (adapter probe), each splint probe also incorporated a “flanking linker sequence” (CCTACCAGTACGACGTATTTAGCAA) (SEQ ID NO: 2) at its 5′ end.

### Adapter-Primer Probe Pre-Treatment

The ^{CNV}K-containing adapter probe ([5Acryd]GCTA [cnvK]ATACGTCGTACTGGTAGG [Inv-dT](SEQ ID NO: 1), ordered from Gene Link with PAGE purification) undergoes rapid photo cross-linking to the complementary strand through an adjacent pyrimidine base upon UV irradiation at about 366 nm for at least 10 minutes. The irradiation process was conducted using the Boekel UV Crosslinker (234100) equipped with 368 nm-wavelength bulbs (Boekel Part Number 920-0307). The adapter to primer probe ratio was maintained at a molar ratio of 5:1.

### Hybridization

1. The probe mixture was heated at 90 °C for 5 minutes and then equilibrated to 37 °C.
2. To prepare 2× hybridization buffer, ribonucleoside vanadyl complex (RVC) was preheated at 55 °C to dissolve, and the remaining reagents were mixed and brought to 37 °C before adding RVC.
3. The oligo mixture and the 2× hybridization buffer were mixed at a 1:1 ratio as a 1× hybridization mixture before use. About 200 μL of the mixture was added per well for 24-well plates, and about 400 μL per well for 12-well plates.
4. The prepared tissue samples were taken from −20 °C and brought to room temperature for 5 minutes. Once at room temperature, the samples were washed twice with PBSTR for 20 minutes each time.
5. The samples were then quenched once with quenching buffer for 20 minutes (7.51 mg/mL glycine (0.1 M) in PBSTR+1:100 yeast tRNA) and washed with PBSTR for 10 minutes.
6. The sample was then incubated in 1× hybridization mixture in a 40 °C humidified oven with shaking and parafilm wrapping for 36 hours.
7. The samples were washed with PBSTR three times for one hour each wash.

| Buffer | Ingredients | Stock Conc. | | Final Conc. | | Amount | |
|---|---|---|---|---|---|---|---|
| PBST | Triton-X 100 | 10% | | 0.1% | | 0.5 | mL |
| | PBS | 1× | | ~1× | | 49.5 | mL |
| | Total | | | | | 50 | mL |
| PBSTR | SUPERase•In | 20 | U/μL | 0.1 | U/μL | 50 | μL |
| (prepare fresh) | PBST | | | | | 10 | mL |
| | Total | | | | | 10 | mL |
| 2× Hybridization buffer | SSC | 20× | | 4× | | 400 | μL |
| (prepare fresh) | Formamide | 100% | | 20% | | 400 | μL |
| | TritonX-100 | 10% | | 2% | | 400 | μL |
| | RVC | 200 | mM | 40 | mM | 400 | μL |
| | Yeast tRNA | 10 | mg/mL | 0.2 | mg/mL | 40 | μL |
| | SUPERase•In | 20 | U/μL | 0.2 | U/μL | 20 | μL |

| | | | | | |
|---|---|---|---|---|---|
| | SDS | 20% | 0.4% | 40 | μL |
| | H₂O | | | 300 | μL |
| | Total | | | 2 | mL |
| High salt wash buffer | SSC | 20× | 4× | 1 | mL |
| | PBSTR | | | 4 | mL |
| | Total | | | 5 | mL |

### Polymerization

1. Prepare 10% VA-044 by dissolving 100 mg VA-044 in 1 mL H₂O.
2. Prepare fresh 10% APS solution by dissolving 50 mg APS solid in 500 μL water.
3. The samples were incubated in monomer buffer (with 0.25% VA-044) at 4 °C for 60 minutes.
4. Then the buffer was aspirated, and 55 μL polymerization mixture was added to the center of the sample and immediately covered by Gel Slick coated coverslip.
5. The polymerization reaction proceeded for 1.5 hours at 40 °C under a N₂ atmosphere, then washed three time with PBSTR for ten minutes each wash.

| Buffer & | | Stock | Final | | |
|---|---|---|---|---|---|
| Reaction | Ingredients | Conc. | Conc. | Amount | |
| Monomer | Acrylamide | 40% | 4% | 1 | mL |
| buffer | Bis-acrylamide | 2% | 0.2% | 1 | mL |
| | SSC | 20× | 2× | 1 | mL |
| | H₂O | | | 6.75 | mL |
| | Tetramethylethylenediamine | 100% | 0.2% | 20 | μL |
| | VA-044 | 10% | 0.25% | 250 | μL |
| | Total | | | 10 | mL |
| Polymer- | Ammonium persulfate | 10% | 0.2% | 10 | μL |
| ization | Monomer buffer | | | 490 | μL |
| mixture | Total | | | 500 | μL |

### Tissue Clearing

1. The tissue-gel hybrids were digested with Proteinase K mixture at 37 °C overnight.
2. Then, the tissue-gel hybrids were washed with PBSTR (at least 4 times) for 30 minutes each wash (to remove SDS thoroughly).

| | | Stock | Final | | |
|---|---|---|---|---|---|
| Reaction | Ingredients | Conc. | Conc. | Amount | |
| Proteinase | 20× SSC | 20× | 2× | 100 | μL |
| K mixture | SDS | 20% | 1% | 50 | μL |
| | Proteinase K | 20 mg/mL | 0.4 mg/mL | 20 | μL |
| | H₂O | | | 830 | μL |
| | Total | | | 1 | mL |

### Ligation

1. After the final wash of PBSTR, the samples were incubated with 400 μL ligation mixture at room temperature overnight.
2. Then, the sample were washed with PBSTR three times for 30 min each.

| Reac- | | Stock | Final | | |
|---|---|---|---|---|---|
| tion | Ingredients | Conc. | Conc. | Amount | |
| Ligation | T4 DNA | 10× | 1× | 40 | μL |
| mixture | ligase buffer | | | | |
| | BSA | 100× | 1× | 4 | μL |
| | SUPERase•In | 20 U/μL | 0.2 U/μL | 4 | μL |
| | T4 DNA ligase | 5 Weiss U/μL | 100 Weiss U/mL | 8 | μL |
| | H₂O | | | 344 | μL |
| | Total | | | 400 | μL |

### Rolling Circle Amplification (RCA)

1. 5-(3-aminoallyl)-dUTP was diluted from 50 mM stock to 4 mM in ultrapure water.
2. The samples were then incubated with 400 μL RCA mixture at 4 °C for 60 minutes and then at 30 °C overnight.
3. The samples were then washed with PBSTR three times for 30 minutes each time.

| | | Stock | | Final | | | |
|---|---|---|---|---|---|---|---|
| Reaction | Ingredients | Conc. | | Conc. | | Amount | |
| RCA | Phi29 buffer | 10× | | 1× | | 40 | μL |
| mixture | dNTP mixture | 10 | mM | 250 | μM | 10 | μL |
| | 5-(3-aminoallyl)- | 4 | mM | 20 | μM | 2 | μL |
| | dUTP | | | | | | |
| | BSA | 100× | | 1× | | 4 | μL |
| | SUPERase•In | 20 | U/μL | 0.2 | U/μL | 4 | μL |

| | | | | | |
|---|---|---|---|---|---|
| Phi29 DNA ligase | 10 | U/μL | 200 Weiss U/mL | 8 | μL |

| | | |
|---|---|---|
| H₂O | 332 | μL |
| Total | 400 | μL |

### BS(PEG) Crosslinking

1. A solution of 5 mM BS(PEG) 9 in PBST was prepared from 250 mM stock (1:50 dilution). Then, the sample was incubated at 5 mM BS(PEG) 9 at 4 °C for 8 hours (or room temperature for a shorter time).
2. The sample was washed with PBST at room temperature for 10 minutes and then quenched with 0.1 M glycine in PBST at room temperature for 30 minutes.
3. The sample was then washed again with PBST at room temperature for 10 minutes.

| Reaction | Ingredients | Stock Conc. | Final Conc. | Amount | |
|---|---|---|---|---|---|
| BS(PEG)₉ | BS(PEG)₉ | 250 mM | 5 mM | 10 | μL |
| | PBST | | | 490 | μL |
| | Total | | | 500 | μL |
| Quench | Glycine | | | 7.51 | mg |
| | PBST | | | 1 | mL |
| | Total | | | 1 | mL |

### Dephosphorylation (Pretreatment for Sequencing)

To minimize background fluorescence, the sample was treated with dephosphorylation mixture for 3 hours or overnight at 37 °C, then washed at least 4 times with PBST for 30 minutes each wash.

| | | Stock | Final | | |
|---|---|---|---|---|---|
| Reaction | Ingredients | Conc. | Conc. | Amount | |
| Dephosphorylation | Antarctic Buffer | 10× | 1× | 100 | μL |
| mixture | BSA | 100× | 1× | 10 | μL |
| | Antarctic enzyme | | | 50 | μL |
| | H₂O | | | 840 | μL |
| | Total | | | 1 | mL |

### Sequencing Reaction

1. Each sequencing cycle began with treating the sample three times with stripping buffer at room temperature for 15 minutes each time.
2. The sample was then washed three times with PBST for 15 minutes for each wash.
3. For each sequencing cycle, the sample was incubated with sequencing mixture at room temperature overnight.
4. The sample was then washed three times with washing and imaging buffer for 10 minutes each wash, and then immersed in washing and imaging buffer for imaging.
5. DAPI staining was performed before Cycle 1 for 3 hours.

| | | Stock | | Final | | | |
|---|---|---|---|---|---|---|---|
| Buffer & reaction | Ingredients | Conc. | | Conc. | | Amount | |
| Stripping buffer | Formamide | 100% | | 60% | | 30 | mL |
| | Triton-X-100 | 10% | | 0.1% | | 500 | μL |
| | H₂O | | | | | 20 | mL |
| | Total | | | | | 50 | mL |
| Sequencing | T4 DNA ligase buffer | 10× | | 1× | | 30 | μL |
| mixture | BSA | 100× | | 1× | | 3 | μL |
| | Reading probe | 100 | μM | 10 | μM | 30 | μL |
| | Fluorescent decoding | 100 | μM | 5 | μM | 15 | μL |
| | probe | | | | | | |
| | T4 DNA ligase | 5 | U/μL | 0.2 | U/μL | 12 | μL |

| | | | | | |
|---|---|---|---|---|---|
| | H₂O | | | 210 | μL |
| | Total | | | 300 | μL |
| Washing and imaging | SSC | 20× | 2× | 5 | mL |
| buffer | Formamide | 100% | 10% | 5 | mL |
| | H₂O | | | 40 | mL |
| | Total | | | 50 | mL |

### Example 3: Three-Dimensional Single-Cell Transcriptomics and Translatomics in Thick Tissue Blocks

Functional heterogeneity in biological tissues arises in part from the diverse gene and protein expression patterns unique to specific cell types, which shape the three-dimensional (3D) molecular architecture and cellular features of different tissues. Single-cell RNA sequencing (scRNA-seq) provides a detailed mapping of the transcriptome, offering insights into gene transcription dynamics at the level of individual cells. Ribosome profiling, on the other hand, maps the translatome, facilitating the analysis of translation across the transcriptome and shedding light on protein synthesis and post-transcriptional regulation. Furthermore, in situ sequencing technologies enable the study of both the transcriptome and translatome within their original spatial contexts. Among these methods, STARmap stands out as an imaging-based, targeted spatial transcriptomics technique capable of simultaneously profiling thousands of gene transcripts¹. RIBOmap is an imaging-based spatial sequencing method for mapping ribosome-bound mRNA, allowing highly multiplexed characterization of protein synthesis within tissues².

Most spatial sequencing techniques are confined to analyzing thin tissue sections (10-20 μm)³. However, many functional and structural studies in neuroscience, such as electrophysiology, activity-based imaging, and 3D morphological analyses, require the use of thick tissue. Thin tissue sections are inadequate for integration with these neuroscience methodologies. Additionally, using serial thin sections followed by alignment is not feasible due to the tissue deformation that occurs during sectioning. Therefore, to effectively integrate molecular or functional characterizations from intact tissue preparations with cellular-resolution transcriptome or translatome readouts from the same samples, thick spatial transcriptomic and translatomic analyses are necessary. This approach not only preserves 3D morphology but also enables readouts from significantly larger cell populations. Consequently, it facilitates a more comprehensive and accurate multimodal characterization, advancing our understanding of complex biological systems at a subcellular level.

Current 3D spatial sequencing approaches are limited in several key aspects, including the number of genes they can analyze, typically fewer than 300, and the size of the imaging areas, often restricted to a single brain region^{1,4-6}. These limitations arise from several challenges. First, an efficient and scalable method is needed to embed a large number of probes targeting diverse genes into the hydrogel matrix for imaging-based 3D spatial sequencing. Second, a robust cDNA crosslinking technique is required to prevent displacement or denaturation of these molecules between imaging rounds, which is caused by buffer-dependent hydrogel expansion and contraction. This displacement leads to a progressively lower signal-to-noise ratio (SNR), complicating spot identification and alignment. Third, an automated computational approach is essential for managing the vast amounts of data generated-multi-terabyte, multi-round, high-resolution imaging of thick tissue specimens. Moreover, current 3D spatial sequencing methods are limited to mapping spatial transcriptomics and lack the capability to map the translatome, thus hindering highly multiplexed characterization of gene translation at single-cell resolution. Addressing these challenges is crucial for advancing the understanding of gene expression and regulation in complex biological systems.

The present disclosure describes thick-STARmap and thick-RIBOmap methods that address the aforementioned challenges by introducing an efficient strategy for embedding probes in a hydrogel, digesting tissue before enzyme reactions, embedding cDNA amplicons in a double-hydrogel network, and modifying existing computational image processing tools to handle large datasets efficiently and consistently. This enables a sequencing-based approach for high-content 3D in situ quantification of thousands of gene transcripts and their respective translation activities within intact thick tissue samples. Utilizing thick-STARmap and thick-RIBOmap, the gene transcription and translation of 1017 genes were profiled in intact mouse brain tissue at 300 nm resolution within a thick hydrogel-tissue scaffold, revealing heterogeneity in gene translation across cell types. Additionally, by combining this method with Tetbow labeling, the transcriptome-resolved neuron morphologies were simultaneously profiled, achieving in situ multimodal mapping in the adult mammalian brain that was previously unattainable. Furthermore, the applicability of the method on human skin cancer was demonstrated, uncovering intriguing tumor-immune interactions with more accurate and quantitative spatial distribution compared to thin tissue analyses.

### Thick-STARmap and Thick-RIBOmap Workflow

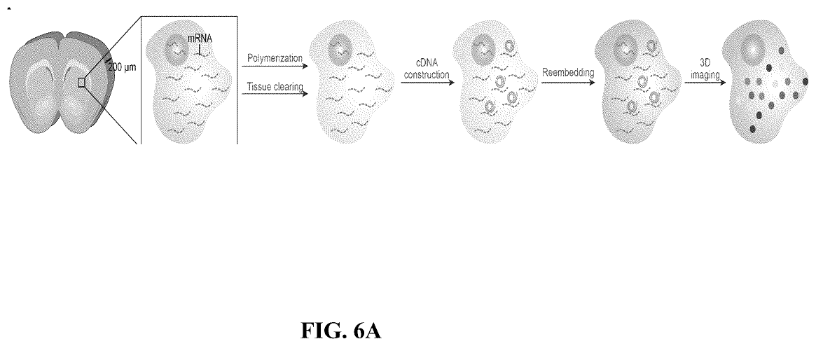

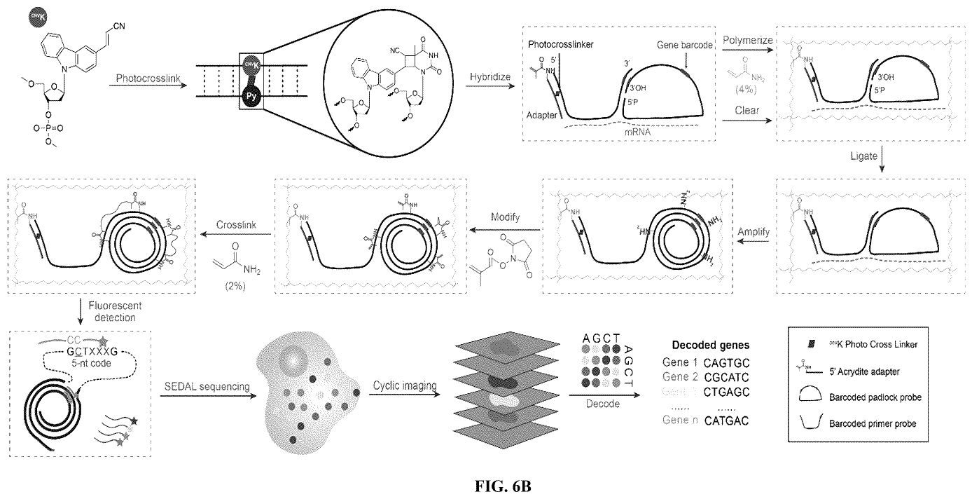

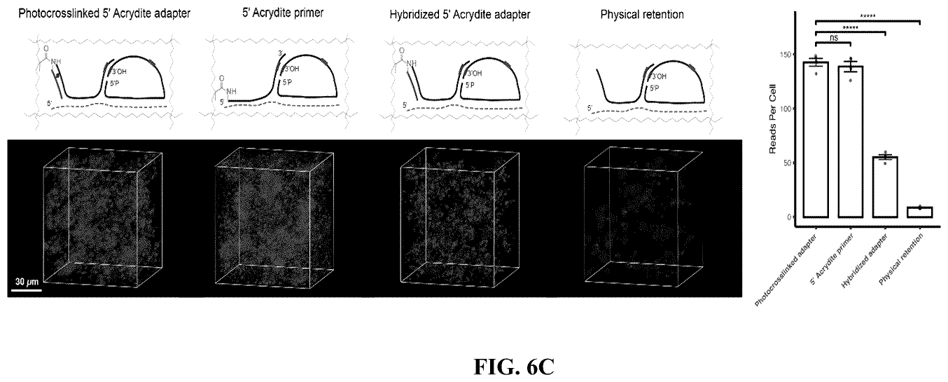

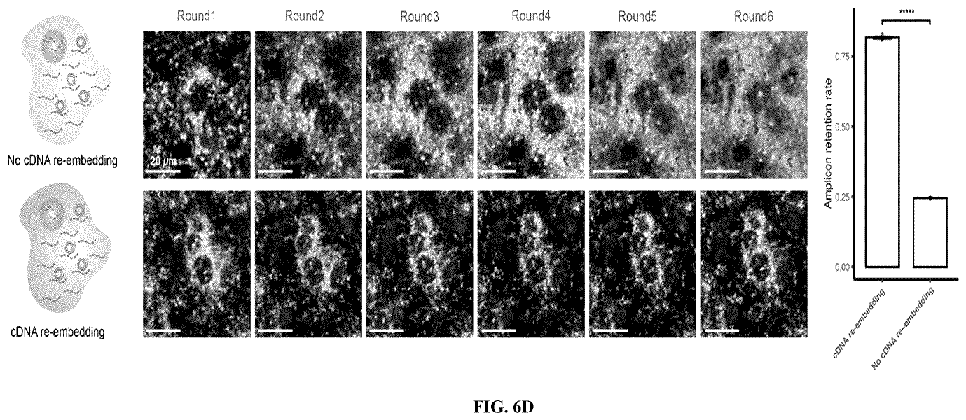

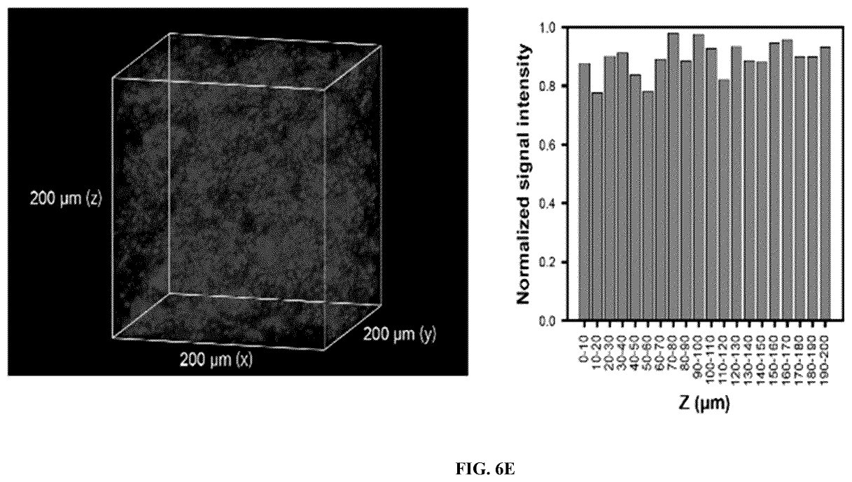

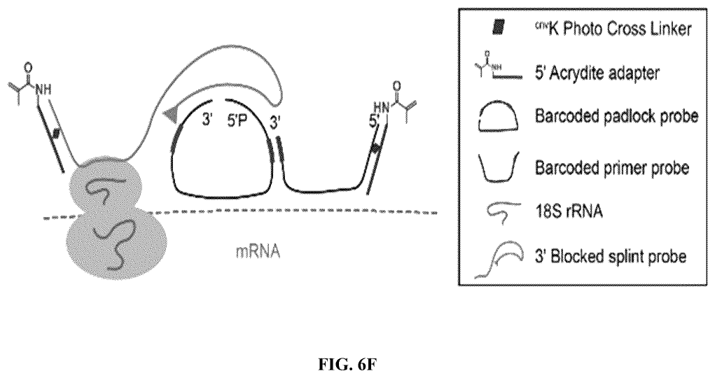

Two imaging-based in situ sequencing methodologies were designed that spatially resolve steady-state RNA (thick-STARmap) and ribosome-bound mRNA (thick-RIBOmap) in thick tissue blocks. Despite differences in probe design, thick-STARmap and thick-RIBOmap follow the same workflow (FIG. 6A). The workflow begins with the hybridization of pre-designed oligonucleotide probe sets to target RNA molecules. Following hybridization, the probes and tissue are embedded within a hydrogel matrix, which is then subjected to protein digestion and lipid removal to enhance enzyme penetration, ensuring sufficient depth coverage in thick tissue samples. Subsequently, in situ cDNA amplicons are synthesized through enzymatic ligation and rolling circle amplification (RCA). Each cDNA amplicon contains a pre-designed gene-specific identifier, which is subsequently decoded through cyclic imaging.

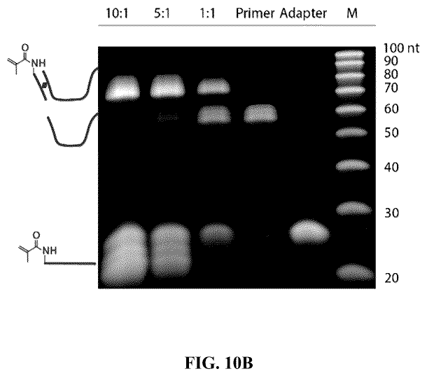

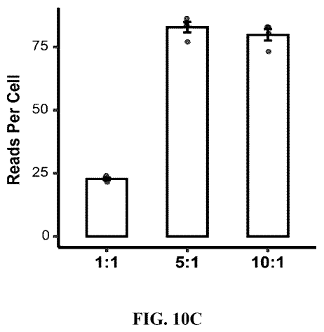

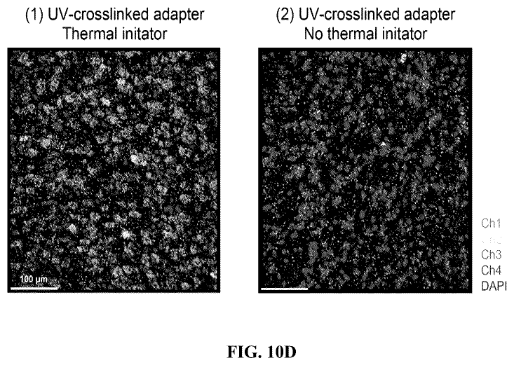

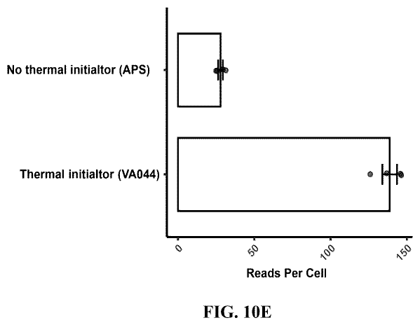

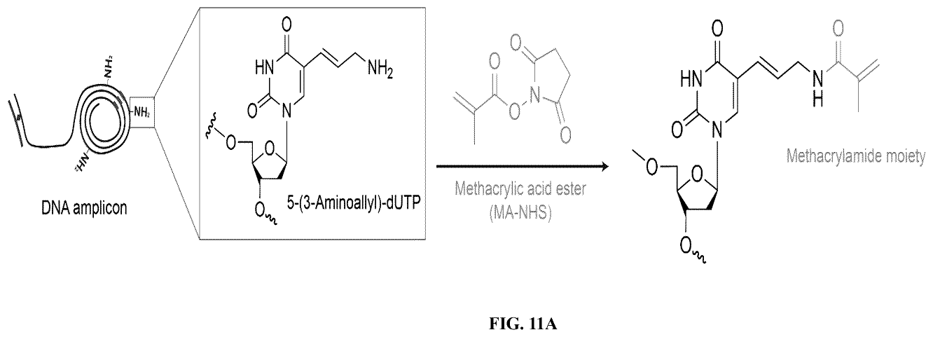

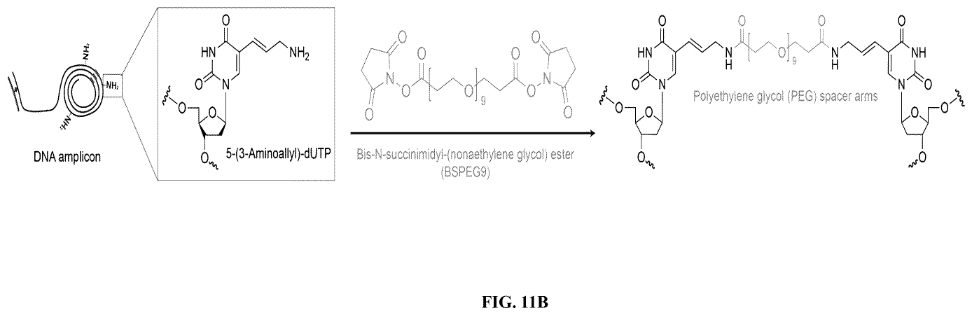

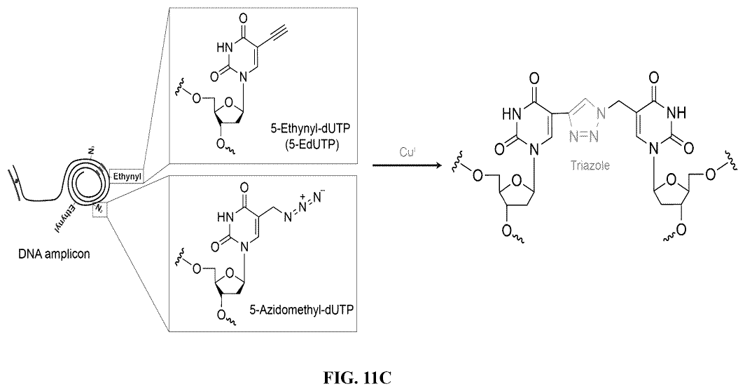

STARmap employs a padlock probe (second probe) to target specific mRNA species of interest, along with a primer (first probe) that binds to the same mRNA transcript adjacent to the padlock probe (second probe) site. Building upon this design, thick-STARmap incorporates a “flanking linker sequence” at the 5′ end of the primer (third probe). This addition enables an acrydite-modified adapter to covalently crosslink with the flanking linker, allowing the probe set to be incorporated into polyacrylamide hydrogels during polymerization (FIG. 6B). Covalent crosslinking is achieved with a nucleoside analog, 3-cyanovinylcarbazole nucleoside (^{CNV}K)⁷, incorporated into the adapter (third probe). Upon 366 nm UV irradiation, the ^{CNV}K-containing adapter undergoes rapid photocrosslinking to the complementary strand via an adjacent pyrimidine base, a process shown to be non-damaging to DNA^{8,9}. Experimental optimization revealed that an adapter-to-primer ratio of 5:1 (i.e., a third probe-to-first probe ratio of 5:1) is sufficient, as higher ratios do not increase the number of amplicons (FIG. 10). Additionally, a 10-minute reaction time achieves nearly 100% crosslinking yield. Notably, using a photocrosslinked 5′ acrydite adapter produces equivalent results to directly modifying the 5′ end of the primer with acrydite. However, the photocrosslinked adapter approach is markedly more efficient and scalable by employing a uniform flanking linker sequence and corresponding adapter for all primers. Due to its high multiplexing capability, this method facilitates the embedding of a large number of probe sets into the hydrogel, expanding the number of targetable RNA species from dozens to thousands. The findings demonstrated that UV crosslinking significantly enhanced probe incorporation efficiency, leading to a higher detection yield of RNA reads (cDNA amplicons) compared to mere hybridization of the adapter (third probe) to the primer (first probe) and substantially surpasses the performance of relying solely on hydrogel physical retention (FIG. 6C).

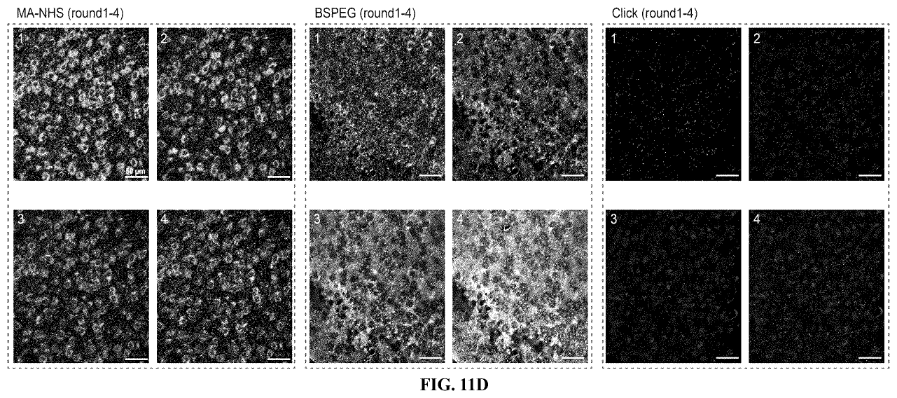

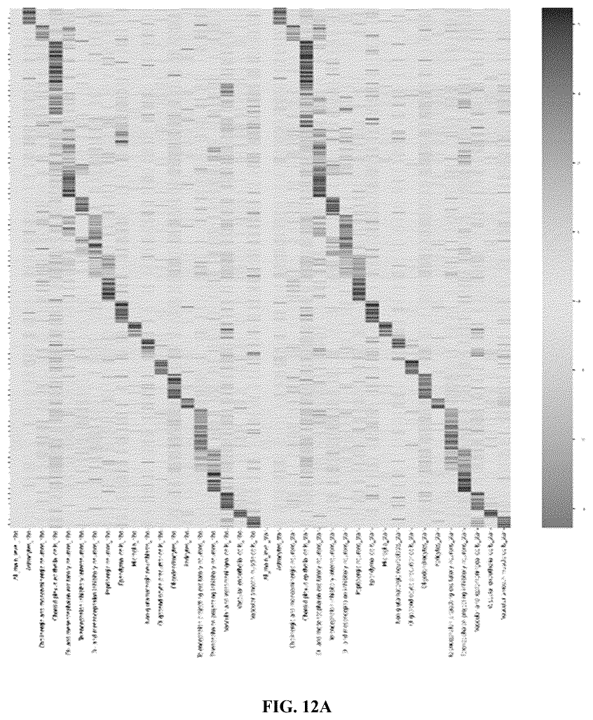

Ammonium persulfate (APS)-initiated polymerization is a rapid process that often results in heterogeneous gel formation, as the gel solidifies before APS can adequately penetrate the central region. To address this limitation, VA-044, a thermal initiator that activates at 40 °C, was incorporated to achieve a more controlled polymerization rate. This modification promotes uniform polymerization throughout the gel, enhancing the crosslinking of probes within the hydrogel (FIG. 11). Following polymerization and protein digestion, enzymatic ligation and rolling circle amplification (RCA) were performed to construct in situ cDNA amplicons. Robust cDNA crosslinking is required for ensuring high-quality in situ sequencing across multiple rounds. Without efficient cDNA crosslinking, cDNA molecules are prone to displacement or denaturation between imaging rounds due to buffer-dependent hydrogel expansion and contraction, resulting in progressively lower SNR (FIG. 6D). To maintain the position and integrity of the amplicons through multiple detection cycles, hydrogel re-embedding was selected over alternative re-embedding techniques. Collectively, the implementation of these strategies specifically devised for thick-STARmap enhanced its robustness and scalability, enabling consistent spatial transcriptomics readouts across 200-μm thick sections of the mouse brain (FIG. 6E).

Insights gained from developing thick-STARmap were leveraged to establish thick-RIBOmap for the investigation of spatial translational control in thick tissue samples. RIBOmap utilizes a tri-probe design strategy to selectively detect and amplify ribosome-bound mRNAs: in addition to the padlock (second probe) and primer (first probe), an additional splint DNA probe (fourth probe) hybridizes to ribosomal RNAs (rRNAs). Building upon this design, thick-RIBOmap incorporates a “flanking linker sequence” at the 5′ end of both the primer and the splint probe (FIG. 6F). The acrydite-modified adapter (third probe) covalently crosslinks with these flanking linkers, enabling the integration of the entire tri-probe set into polyacrylamide hydrogels during polymerization.

Thick-STARmap and Thick-RIBOmap in Tetbow Mouse Brain with 1017 Genes

To evaluate the scalability of thick-STARmap and thick-RIBOmap for high-content 3D intact-tissue transcriptomic and translatomic sequencing, thick-STARmap and thick-RIBOmap were applied to thick mouse brain sections from Tetbow mice, targeting a curated list of 1017 genes. This gene list was compiled from reported cell-type marker genes in adult mouse CNS single-cell RNA sequencing (scRNA-seq) datasets and spatial transcriptomic mouse brain atlas mappings¹⁰⁻¹³. Gene identities encoded by five-nucleotide sequences on the SNAIL probes were read out through six rounds of sequencing by ligation with error rejection (SEDAL).

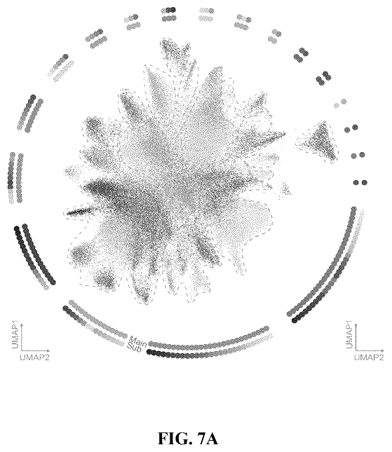

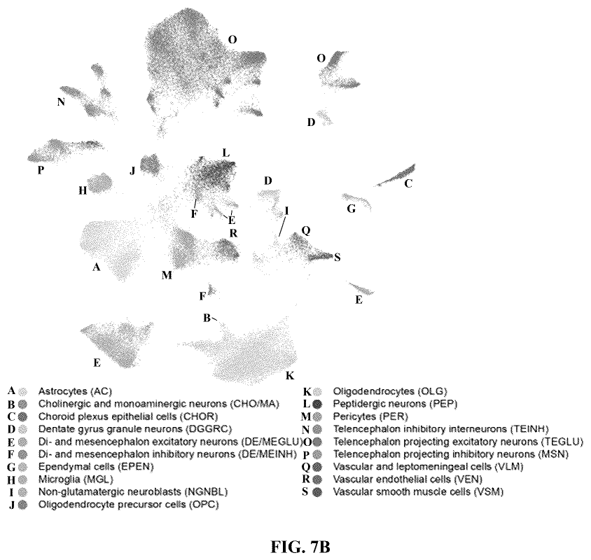

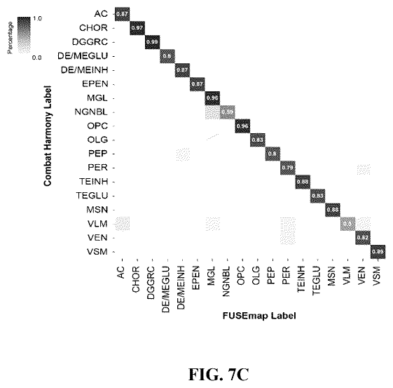

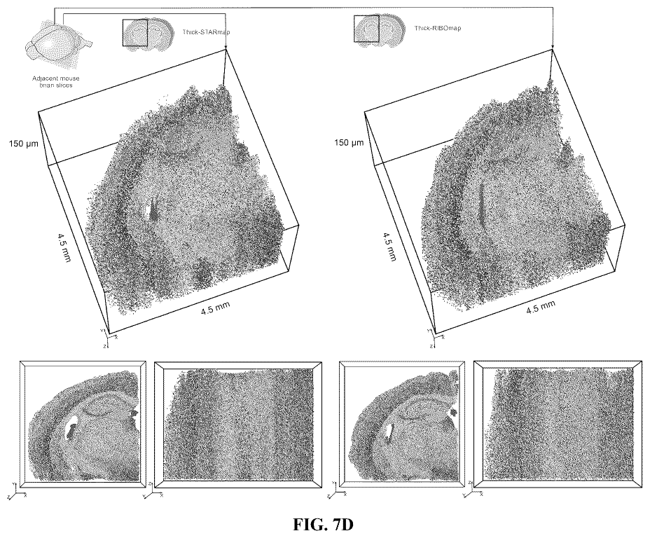

Thick-STARmap and thick-RIBOmap mapping were performed pairwise on adjacent coronal sections of the mouse hemisphere, encompassing multiple brain regions (198,675 cells for thick-STARmap and 164,029 cells for thick-RIBOmap). The spatial transcription and translation patterns of well-known cell-type marker genes and neurotransmitter genes aligned well with previously published spatial transcriptomic and translatomic sequencing results. To annotate cell types and align them with established nomenclature, a mouse brain spatial atlas was utilized as well as a newly developed spatial integration computational method, FUSEmap¹⁴ (FIG. 7A). A key feature of FUSEmap is its consideration of spatial location alongside the gene-expression matrix during integration. FUSEmap integration, followed by nearest-neighbor label transfer, identified 19 main cell types, including 9 neuronal, 5 glial, 1 immune, and 4 vascular cell clusters, all of which exhibited canonical marker genes and expected spatial distributions. Further hierarchical clustering within each main cluster resulted in 137 subclusters. These major and subcluster annotations were consistent with the previously published brain atlas dataset^{13,15,16} To benchmark FUSEmap's performance and cell-type annotation accuracy, integration was performed using the traditional method, Harmony¹⁷, which considers only single-cell gene expression profiles without spatial information (FIG. 7B). The confusion matrix of major cell type assignments showed that FUSEmap's cell types were highly concordant (82.4% matched labels) with those identified by the traditional single-cell sequencing integration method (FIG. 7C), further validating FUSEmap's accuracy in cell type annotation.

Based on these cell typing results, spatial cell maps were generated from the imaged hemibrain region (FIG. 7D). The analysis demonstrated consistent cell typing between thick-STARmap and thick-RIBOmap in terms of gene expression, cell-type composition, and spatial distribution of cell types. Moreover, these spatial cell-type maps were in strong agreement with previously published spatial atlas datasets.

### Comparison of Spatial Translatome and Transcriptome in the Mouse Brain

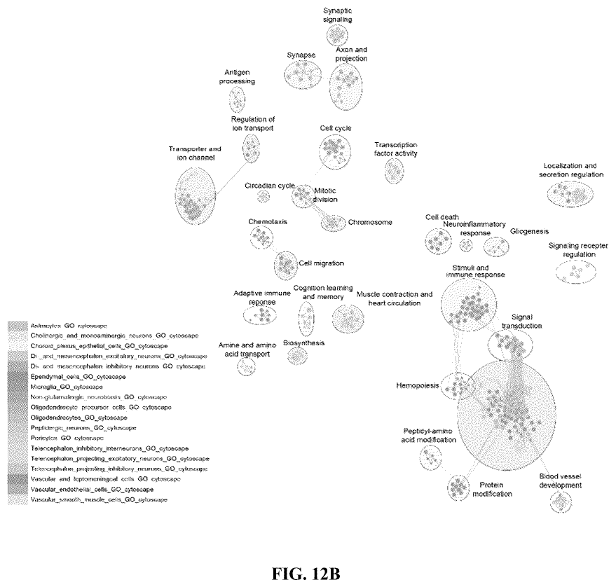

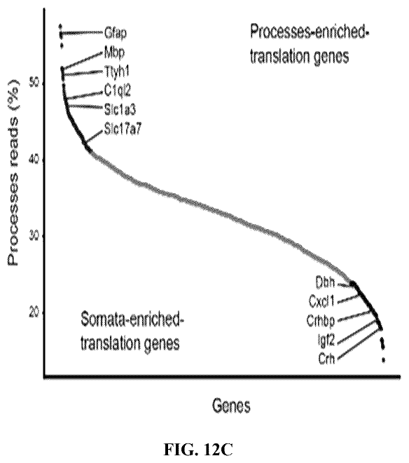

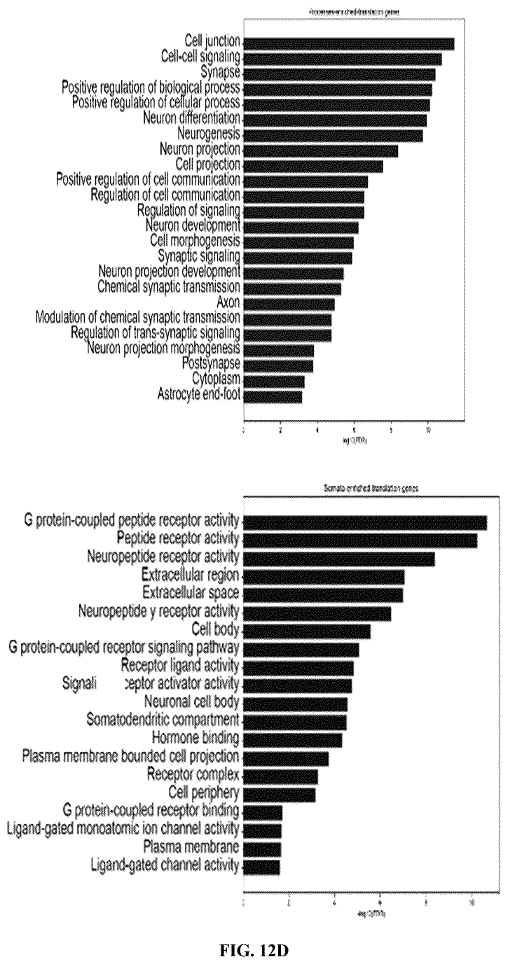

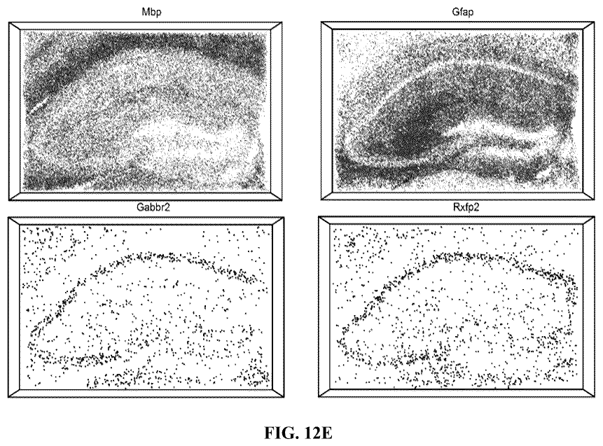

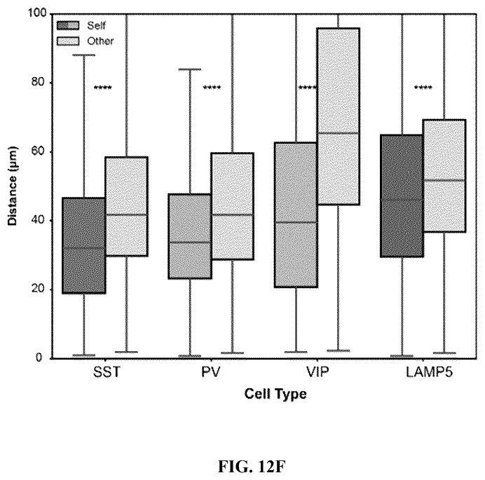

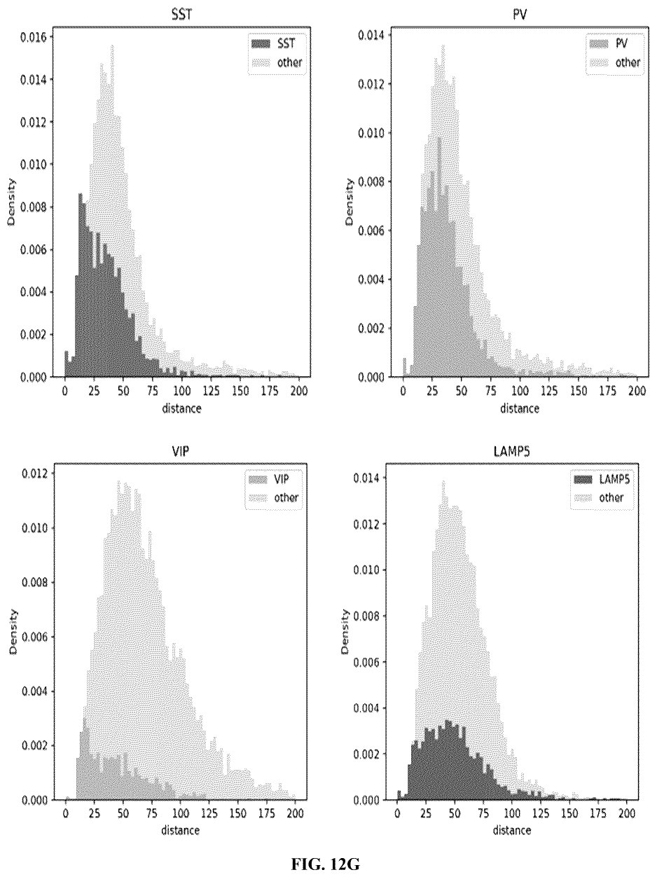

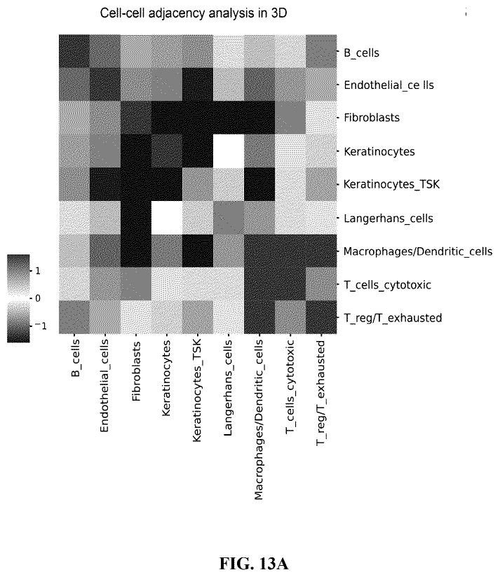

By exploiting the single-cell and spatial resolution of paired thick-STARmap and thick-RIBOmap datasets, the heterogeneity in translational regulation across various cell types and brain regions was probed. Research has previously shown that non-neuronal cells, particularly oligodendrocytes, exhibit significant translational regulation, as evidenced by their low correlation scores between translatome and transcriptome. This discrepancy highlights the regulatory divergence between these molecular profiles. To investigate translationally regulated genes across different cell types, gene clustering was performed using thick-STARmap and thick-RIBOmap profiles, identifying 18 gene modules (FIG. 12A) with distinct functions and expression patterns (FIG. 12B).

Beyond the extensive anatomical analysis of the brain, investigating translational control at the subcellular level was observed. Translation localized to the soma and peripheral branches (processes) in brain tissue plays a pivotal role in the organization and adjustment of neuronal and glial networks in response to physiological stimuli during neurodevelopment and memory formation. To dissect this localized translation, thick-RIBOmap reads were categorized into somata reads (within the cell body, identified using Watershed 3D) and processes reads (the rest of the reads). Then the top 10% of genes were identified with the highest and lowest processes-to-somata ratios, designating them as enriched in processes and somata, respectively (FIG. 12C). GO analysis indicated that genes enriched in processes are involved in the cytoplasm, cell projection, cell junction, and cell-cell signaling, while those enriched in somata are associated with the extracellular matrix and various receptors (FIG. 12D). A significant concentration of processes-enriched translation signals in the hippocampal neuropils were observed, highlighting the critical role of localized translation in these brain regions.

To elucidate finer volumetric patterns, a detailed analysis was performed of the nearest-neighbor distances among various interneuron subtypes. Prior studies have demonstrated that interneurons of identical subtypes frequently form juxtaposed pairs in the mouse visual cortex. Here, the result substantiates these findings, indicating that an inhibitory neuron is predominantly adjacent to another of the same subtype (Lamp5, Vip, Sst, or Pvalb) rather than other inhibitory subtypes (FIGS. 12F and 12G).

### Characterizing the Morphological Features of Transcriptomic Types

Understanding the brain function necessitates a detailed mapping of its neuroanatomy. Electron microscopy (EM) remains the gold standard for neuroanatomy studies due to its unmatched nanometer resolution, indispensable for clearly identifying synapses^{18,19} However, EM reconstructions frequently lack molecular information needed to determine cell types, as transcripts or protein identification is rare with EM alone. Additionally, the current analytical throughput is inadequate for studying long-range projections. The integration of stochastic multicolor labeling techniques with spatial transcriptomic mapping offers a promising solution. This combined approach enables the generation of comprehensive, transcriptome-resolved projection diagrams of individual neurons within densely labeled neural populations.

To exploit the unique advantages of thick tissue mapping and simultaneously interrogate transcriptomic readouts and morphology within the same brain section, the stochastic multicolor genetic labeling tool, Tetbow²⁰, was integrated into the workflow (FIG. 8A). Tetbow enables comprehensive and high-resolution mapping of intermingled neurons in situ by tagging individual neurons with stochastic combinations of three fluorescent proteins. It has also been demonstrated that systemically delivered AAVs allow a more uniform distribution of labeled cells and color diversity. Thus, the AAV-PHP.eB²¹ variant was utilized to co-administer three separate vectors encoding the fluorescent proteins, along with a tTA expression vector. This AAV-PHP.eB variant has been demonstrated to transduce the central nervous system, thereby facilitating widespread fluorescent protein expression across the brain (FIG. 8B).

After tissue sectioning and embedding the probe sets into the hydrogel through polymerization, the three Tetbow fluorescent proteins along with DAPI were imaged to reveal neuronal morphology. These fluorescent proteins were subsequently digested during the protein digestion step. Following this, cDNA amplicons were constructed and sequenced as previously described. Furthermore, DAPI was co-stained to serve as a fiducial marker for image registration between the two modalities so that each Tetbow neuron can be identified by its molecular subtype.

Each neuron's dendritic morphology was reconstructed using Imaris. A total of 50 neurons in the imaged volume were reconstructed across 40 molecular subtypes (FIG. 8C). In agreement with past findings, the dendritic morphologies were resolved of typical pyramidal neurons in different brain regions including CA1 hippocampal and layer V neocortical pyramidal neurons. The morphology of glutamatergic pyramidal neurons was better preserved in thick slices than thin slices since these neurons have long-range projections. As expected, the most prominent dendritic structure of pyramidal neurons was resolved: the apical dendrite, which in CA1 hippocampal neurons extends towards the stratum lacunosum-moleculare, while in layer V neocortical neurons extend towards the cortical surface, both branching and forming tree-like structures. Although cortical GABAergic neurons constitute a minority of the total neocortical neuronal population, they exhibit a wide variety of dendritic morphologies. In this study, the morphological diversity of several major subclasses of GABAergic neurons were elucidated, classified by their transcriptomic profiles, within the mouse cerebral cortex.

### Thick-STARmap in Human Cutaneous Squamous Cell Carcinoma

It was investigated whether thick-STARmap could be used to study cell types and interactions in human cancer. Cutaneous squamous cell carcinomas (cSCC) arise from keratinocytes, the major cell type of the epidermis. CSCC are the second-most prevalent form of skin cancer, with over 1 million new cases diagnosed annually in the United States²². Surgical excision is curative in most patients, however an estimated 3.7% of cSCC cases lead to metastatic disease and 1.5% of cases result in death from disease²².

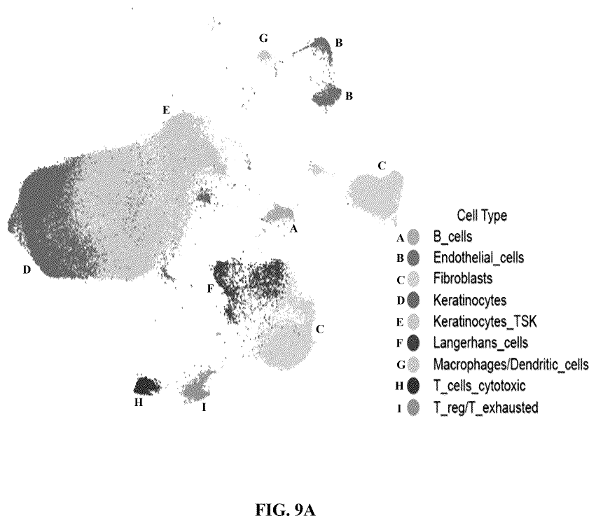

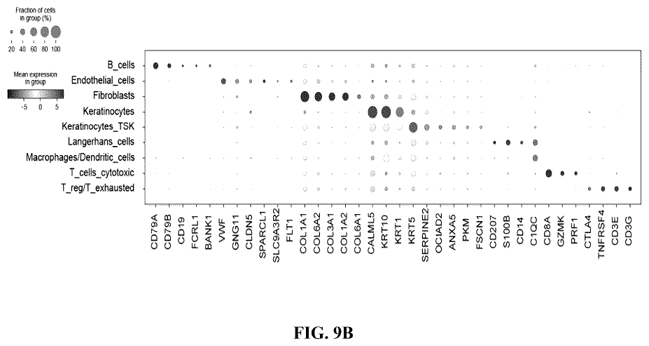

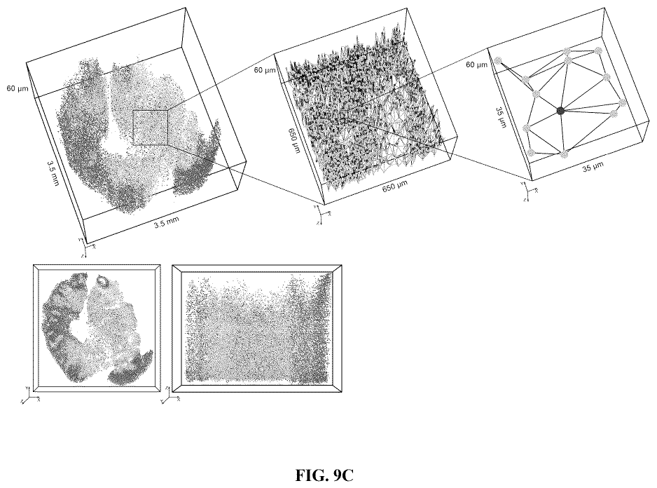

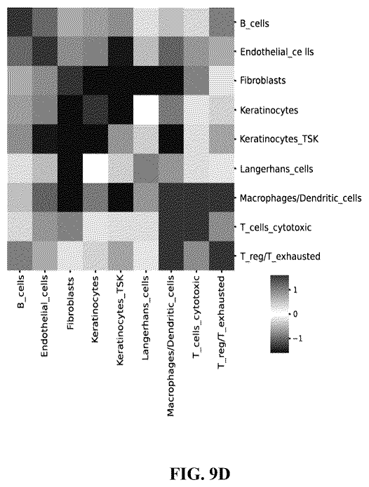

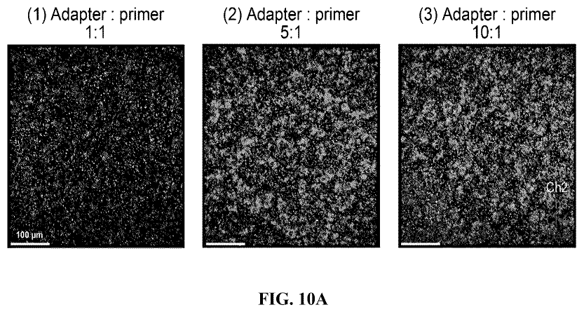

A list of 254 genes from previously published scRNA-seq studies of normal skin and 23-26 was generated. skin cancers, including markers for common skin and immune cell types²³⁻²⁶ Thick-STARmap was performed on a 60 μm thick section of human cSCC obtained from Mohs micrographic surgery (MMS), a sample that included both cSCC tumor and adjacent normal skin. Gene identities encoded by four-nucleotide sequences on the SNAIL probes were read out through five rounds of SEDAL. Following cell segmentation in 3D, data processing, and integration with a published cSCC scRNA-seq dataset (Methods), cells were represented on the UMAP space based on single-cell RNA expression (FIG. 9A). Nine cell types were identified using known marker genes: keratinocytes, tumor-specific keratinocytes, fibroblasts, endothelial cells, B cells, Langerhans cells, macrophages/dendritic cells, cytotoxic T cells, and regulatory T cells/exhausted T cells (FIGS. 9A and 9B). Thick-STARmap enabled dissection of tumor spatial organization at single-cell resolution. Consistent with histologic tumor spatial patterns noted at the time of MMS, tumor-specific keratinocytes in this sample were primarily localized to the center of the tissue while non-tumor keratinocytes were localized to normal skin at the sample periphery (FIG. 9C).

Mapping Cell-Cell Interactions in Human cSCC

The leading risk factor for cSCC is chronic ultraviolet radiation (UVR) exposure, which has mutagenic effects in the skin. UVR-induced somatic mutations translate to a large burden of tumor neoantigens that are thought to be responsible for the high immunogenicity of cSCCs²⁷. Of note, immunosuppressed patients are at a 65-100 fold higher risk of developing cSCC and are significantly more likely to be diagnosed with multiple and metastatic cSCCs^{28,29}. Thus, the present disclosure assesses the spatial organization and potential interactions between tumor and immune cells in the tumor microenvironment.

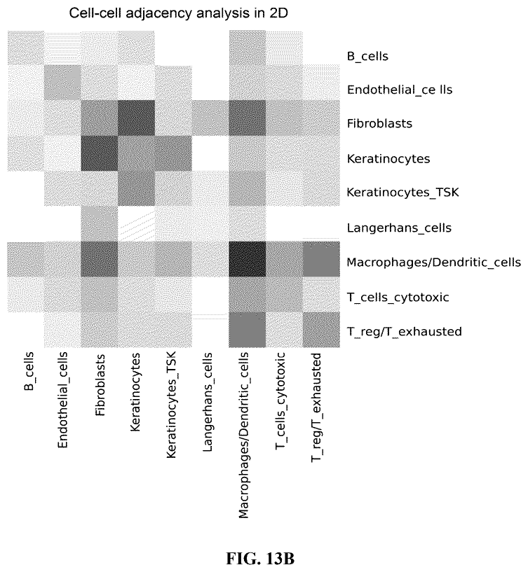

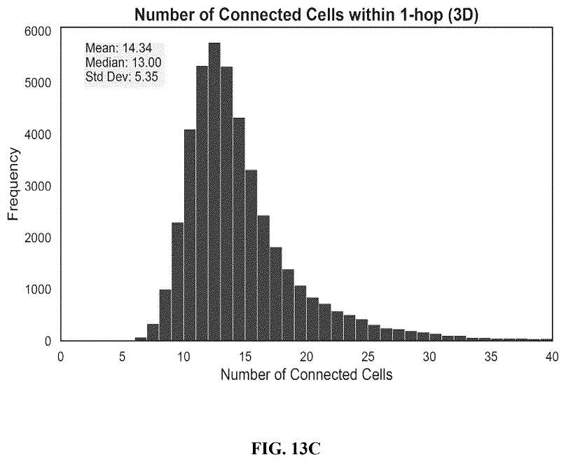

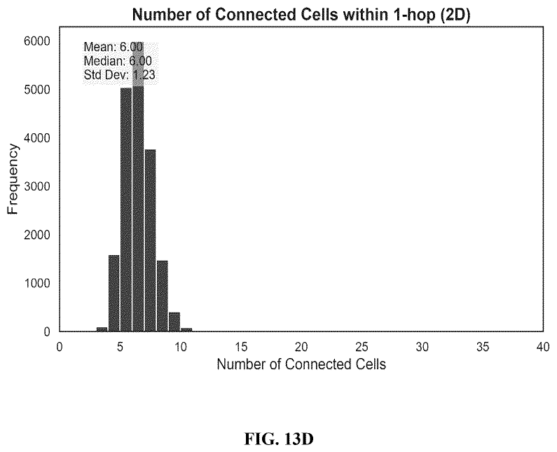

To characterize cell-cell interactions, a mesh graph was generated via Delaunay triangulation of cells, and a near-range cell-cell adjacency matrix from spatial connectivity was computed as previously described^{30,31}. This allowed the nearest neighbors of each cell to be identified and the number of edges between cells of each type with cells of the same or other cell types to be quantified. A heat map of cell type frequencies among first-tier neighbors revealed clear patterns of cell type specific cell-cell communication (FIG. 9C). The same analysis was performed on a pseudo-thin 15 μm thick section of the cSCC sample (FIGS. 13A and 13B). As expected, more cell-cell interactions were detected in thick tissue (mean of 14.3 connected cells) compared to pseudo-thin tissue (mean of 6 connected cells) (FIGS. 13C and 13D).

Across the cSCC sample, strong interactions were detected between cells of the same type, with more same cell type interactions identified in thick tissue compared to pseudo-thin tissue. Similarly, immune cell interactions with other immune cell types such as macrophages/dendritic cells with T cells were more strongly detected in thick compared to pseudo-thin tissue (FIGS. 13A and 13B). Tumor-specific keratinocytes only interacted strongly with two cell types: tumor-specific keratinocytes or Langerhans cells (FIGS. 9C and 9D). This was again more strongly detected in thick tissue than pseudo-thin tissue, demonstrating that the additional 3D morphologic information provided by thick-STARmap increases sensitivity for cell-cell interactions.

Langerhans cells (LCs) are the major resident antigen-presenting cells of the skin and are known to interact with keratinocytes via E-cadherin. LCs have been reported to encounter cSCC cells prior to other dendritic cell subtypes³² and stimulate cytotoxic CD8 T cells and NK cells more efficiently than other DC subsets³³. In the cSCC sample, LCs interacted with T cells and tumor-specific keratinocytes but not normal keratinocytes outside the tumor (FIG. 9C). Taken together, the thick-STARmap cSCC data identified a biologically relevant interaction between tumor-specific keratinocytes and Langerhans with more accurate and quantitative spatial distribution compared to thin tissue analyses.

### Example 4: Materials and Methods

### Mouse Lines

All animal procedures adhered to the care guidelines approved by the Institutional Animal Care and Use Committee (IACUC) of the Broad Institute of MIT and Harvard, under animal protocol #0255 Aug. 19. For the experiments, C57/BL6 mice aged between 6 to 10 weeks were procured from The Jackson Laboratory (JAX).

### Human Cutaneous Squamous Cell Carcinoma Samples

Human cutaneous squamous cell carcinoma tissue was obtained from deidentified discarded hospital specimens approved under the Massachusetts General Hospital Research Committee/IRB protocol #2013P000093.

### Tetbow AAV Injections

The AAV plasmids utilized in this study include pAAV-TRE-m Turquoise2-WPRE (Addgene #104110), pAAV-TRE-EYFP-WPRE (Addgene #104111), pAAV-TRE-tdTomato-WPRE (Addgene #104112), and pAAV-ihSyn1-tTA (Addgene #99120). Tetbow components were packaged into AAV.PHP.eB, which is described in Chan, K. Y. et al., Engineered AAVs for efficient noninvasive gene delivery to the central and peripheral nervous systems. Nat. Neurosci. 20, 1172-1179 (2017), which is incorporated herein by reference. In brief, for each capsid, HEK 293T cells (ATCC CRL-3216) were transfected with a combination of pAAV plasmid and two AAV packaging plasmids (kiCAP-AAV-PHP.eB and pHelper) in a 1:4:2 weight ratio, using polyethylenimine, with a total of 40 μg of DNA per 150-mm culture dish. Fluorescence expression, when applicable, was evaluated via microscopy, and the media was refreshed 20-24 hours post-transfection. Viral particles were collected 72 hours post-transfection from both the cells and the medium by centrifugation, forming cell pellets. These cell pellets were then resuspended in a buffer containing 500 mM NaCl, 40 mM Tris, 10 mM MgCl₂, pH ~10 and 100 U/mL of salt-activated nuclease (SAN, 25 U/μL, Arcticzymes, 70910-202) and incubated at 37 °C for 1.5 hour. Following incubation, the cell lysates were subjected to centrifugation at 2,000 g to remove cellular debris. The viral particles were then isolated through a series of iodixanol gradient steps (15%, 25%, 40%, and 60%). Viruses were collected from both the 40/60% interface and the 40% iodixanol layer. The concentration of the viral particles and buffer change were achieved using Pierce™ Protein Concentrators (Thermo Scientific, 88528), and they were subsequently suspended in sterile phosphate-buffered saline (PBS). To quantify viral titers, viral genomes were measured using quantitative PCR (qPCR). The procedure included treating samples with DNase I (Roche Diagnostics, 4716728001) to eliminate non-packaged DNA and subsequently with proteinase K (Roche Diagnostics, 03115828001) to digest the viral capsid, thereby exposing the viral genomes for qPCR quantification. A linearized genome plasmid served as the reference standard. The viral titers for tTA, tdTomato, EYFP, and m Turquoise2 were 2.15×10¹³, 2.31×10¹³, 3.04×10¹³, and 2.63×10¹³ vg/ml, respectively.

Intravenous administration of AAV.PHP.eB mixture (1×10¹¹ vg tTA, 3.33× 10¹¹ vg tdTomato, 3.33×10¹¹ vg EYFP, 3.33×10¹¹ vg mTurquoise2) was performed via injection into the retro-orbital sinus of adult female C57BL/6 mice (8-10 weeks of age). Twenty-eight days post-injection, the mice were anesthetized with isoflurane. Transcardial perfusion was carried out, initially with 50 mL of cold PBS, followed by 50 mL of 4% PFA. The entire brain was then post-fixed in 4% PFA at 4 °C for 3 hours. Subsequently, the brain was washed multiple times with PBS and placed in a 30% sucrose solution (in PBS) at 4 °C overnight or until it had sunk. Finally, the brain was embedded in O.C.T. (Fisher, 23-730-571) and frozen in liquid nitrogen and stored at −80 °C. Tissue sections, either 100 μm or 200 μm in thickness, were prepared and carefully transferred into pretreated glass-bottom plates.

### Thick-STARmap and Thick-RIBOmap Probe Design

The thick-STARmap and thick-RIBOmap padlock and primer probes were developed based on the methodologies outlined in Wang et al. and Zeng et al., with specific modifications. Each thick-STARmap and thick-RIBOmap primer incorporated a “flanking linker sequence” (CCTACCAGTACGACGTATTTAGCAA) (SEQ ID NO: 2) at the 5′ end to enable hybridization with an acrydite-modified oligonucleotide. The thick-RIBOmap additionally required a splint probe, composed of three segments: a 25-nucleotide sequence at the 5′ end complementary to the 18S ribosomal RNA (rRNA), a stretch of 50 deoxyadenosine nucleotides (dA), and a 12-nucleotide padlock template at the 3′ end. To prevent the 3′ terminus of the splint probes from serving as a RCA primer, a 3′ Inverted dT modification was included. Additionally, each splint probe incorporated a “flanking linker sequence” (CCTACCAGTACGACGTATTTAGCAA) (SEQ ID NO: 2) at the 5′ end to facilitate the hybridization process with the acrydite-modified oligonucleotide.

### Adapter and Primer Pre-Treatment

### The ^{CNV}K-Containing Adapter

([5Acryd]GCTA [^{cnv}K]ATACGTCGTACTGGTAGG [Inv-dT](SEQ ID NO: 1), ordered from Gene Link with PAGE purification) undergoes rapid photo cross-linking to the complementary strand through an adjacent pyrimidine base upon UV irradiation. The irradiation process was conducted using the Boekel UV Crosslinker (234100) equipped with 368 nm-wavelength bulbs (Boekel Part Number 920-0307). The adapter to primer was maintained at a molar ratio of 5:1.

### Thick-STARmap and Thick-RIBOmap Protocol

Glass-bottom 12-well plates (Mattek, P12G-1.5-14-F) were treated with oxygen plasma using the Anatech Barrel Plasma System at 100W and 40% O₂ for 5 min. Following this, the plates were immersed in a 1% methacryloxypropyltrimethoxysilane (Bind-Silane) solution for 60 min at room temperature. The plates then underwent three consecutive ethanol washes and were allowed to air dry. Subsequently, a 0.1 mg/mL Poly-D-lysine solution was applied to the plates for 1 hour, followed by three rinses with distilled water.

Tissue slices were transferred and adhered to pre-treated glass-bottom 12-well plates. The samples were permeabilized using 1 mL of pre-chilled methanol at −20 °C for one hour. During this period, PBST solution, comprising 0.1% Triton-X 100 in PBS, was prepared. The samples were then washed with 500 μL of PBSTR (0.1 U/mL SUPERase·In in PBST) for 30 min. This was followed by a quenching step with 500 μL of quenching solution (1 mg/mL Yeast tRNA, 100 mM Glycine in PBSTR) at room temperature for 30 minutes, followed by another 30-min wash with PBSTR. Subsequently, hybridization buffers were prepared. The base composition of the hybridization buffer included 2×SSC, 10% formamide, 1% Triton-X 100, 20 mM RVC, 0.1 mg/mL yeast tRNA, 0.1 U/μL SUPERase. In, and 0.2% SDS. For the thick-STARmap samples, this buffer was supplemented with pooled thick-STARmap padlock and pre-treated primer at a concentration of 5 nM per oligo. For the thick-RIBOmap samples, the hybridization buffer additionally contained 100 nM of pre-treated splint probe for RIBOmap. The samples were incubated in 300 μL of hybridization buffer in a 40 °C humidified oven with gentle shaking for 36 hours. After incubation, the samples were washed for 30 min with PBSTR, followed by a 30-min wash in high salt buffer (4×SSC in PBSTR) at 37 °C. Finally, the samples were washed once more with PBSTR at 37 °C.

To cast the tissue-hydrogel hybrid, the samples were first incubated with monomer buffer (4% acrylamide, 0.2% bis-acrylamide, 2× SSC) supplemented with 0.2% TEMED and 0.25% VA-044 at 4 °C for 60 min. Following incubation, the buffer was aspirated, and 55 μL of a polymerization mixture (0.2% TEMED, 0.2% ammonium persulfate, and 0.25% VA-044 in monomer buffer) was added to the center of the sample and immediately covered with a Gel Slick-coated coverslip. The polymerization process was conducted in a 40 °C. N₂ oven for 90 min. Subsequently, the sample was washed with PBSTR three times for 15 min each. For Tetbow mice samples, the tissue was stained with DAPI for 3 hours and then immersed in a washing and imaging buffer (10% formamide in 2× SSC buffer) containing 0.1 U/μL SUPERase·In RNase inhibitor. Confocal images of Tetbow fluorescent proteins (tdTomato, EYFP, and mTurquoise2) and DAPI were acquired using an inverted confocal microscope, Leica TCS SP8 (version 3.5.5.19976), equipped with a 405 nm and 442 nm diode, a white light laser, HyD detectors, and a 25× water-immersion objective (NA 0.95). The voxel size for imaging was 0.32 μm×0.32 μm×0.70 μm. The following wavelengths were used for imaging: 405 nm for DAPI, 442 nm for m Turquoise2, 506 nm for EYFP, and 550 nm for tdTomato.

The tissue-gel hybrids were then digested with 1 mL Proteinase K mixture (0.4 mg/mL Proteinase K in 2× SSC and 1% SDS) at 37 °C for overnight, then washed by PBSTR 3 times for 30 min each. The sample was then incubated in ligation mixture (0.25 U/μL T4 DNA ligase, 1:100 BSA, 0.2 U/μL SUPERase·In RNase inhibitor) at room temperature overnight with gentle shaking and then washed with PBSTR three times for 30 mins each. Then the sample was incubated with 400 μl rolling-circle amplification mixture (0.5 U/μL Phi29 DNA polymerase, 250 μM dNTP, 20 μM 5-(3-aminoallyl)-dUTP, 1:100 BSA and 0.2U/μL of SUPERase In RNase inhibitor in 1× Phi29 buffer) at 4 °C for 60 min for equilibrium before incubating at 30 °C for 8-14 hours for amplification and then washed with PBST 3 times for 30 mins each. The samples were then treated with 20 mM methacrylic acid N-hydroxysuccinimide ester in 100 mM sodium bicarbonate buffer for 4 hours to overnight at room temperature. Following the exact same procedures casting tissue-hydrogel hybrid, cDNA amplicons were re-embedded with 2% acrylamide, 0.05% bis-acrylamide to enable cDNA amplicon crosslinking in the tissue-hydrogel setting, and such cross-linking is essential to maintain the position and integrity of the amplicons through many cycles of detection. Samples were stored in PBST or wash and imaging buffer at 4 °C until imaging and sequencing.

Before SEDAL, the samples were treated with the dephosphorylation mixture (0.25 U/μL Antarctic Phosphatase, 1× BSA, in 1× Antarctic Phosphatase buffer) at 37 °C for 4 hours and washed by PBST three times for 30 min each. Each sequencing cycle began with treating the sample three times, 15 min each, with the stripping buffer (60% formamide and 0.1% Triton X-100 in water) at room temperature, followed by washing with PBST three times for 15 min each. Then the samples were incubated with a at minimal 300 μL sequencing-by-ligation mixture (0.2 U/μL T4 DNA ligase, 1× BSA, 10 μM reading probe, and 5 μM fluorescent decoding oligonucleotides in 1× T4 DNA ligase buffer) at room temperature for overnight, followed by rinsing with washing and imaging buffer three times for 10 min each before imaging. Images were acquired using the same Leica TCS SP8 with a 25× water-immersion objective (NA 0.95). The voxel size for imaging was 0.32 μm×0.32 μm×0.70 μm. For each round, images were acquired with Alexa 488, 546, 594 and 647 illumination. DAPI was dissolved in wash and imaging buffer and used for nuclei staining for 3 hours before the first round. The DAPI signal was collected at the first cycle of imaging with additional 405 nm wavelength. Six cycles of imaging were performed to detect 1017 genes.

### REFERENCES FOR EXAMPLE 1

1. Wang, X.; Allen, W. E.; Wright, M. A.; Sylwestrak, E. L.; Samusik, N.; Vesuna, S.; Evans, K.; Liu, C.; Ramakrishnan, C.; Liu, J.; Nolan, G. P.; Bava, F.-A.; Deisseroth, K. Three-Dimensional Intact-Tissue Sequencing of Single-Cell Transcriptional States. Science 2018, 361 (6400).
2. Zeng, H.; Huang, J.; Ren, J.; Wang, C. K.; Tang, Z.; Zhou, Y.; Aditham, A.; Shi, H.; Sui, X.; Wang, X. Spatially Resolved Single-Cell Translatomics at Molecular Resolution. Science 2023, 380 (6652).
3. Yoshimura, Y.; Fujimoto, K. Ultrafast Reversible Photo-Cross-Linking Reaction: Toward in Situ DNA Manipulation. Org. Lett. 2008, 10 (15), 3227-3230.
4. Yoshimura, Y.; Ohtake, T.; Okada, H.; Fujimoto, K. A New Approach for Reversible RNA Photocrosslinking Reaction: Application to Sequence-Specific RNA Selection. Chembiochem 2009, 10 (9), 1473-1476.
5. Fujimoto, K.; Konishi-Hiratsuka, K.; Sakamoto, T.; Yoshimura, Y. Site-Specific Cytosine to Uracil Transition by Using Reversible DNA Photo-Crosslinking. Chembiochem 2010, 11 (12), 1661-1664.
6. Chen, R.; Blosser, T. R.; Djekidel, M. N.; Hao, J.; Bhattacherjee, A.; Chen, W.; Tuesta, L. M.; Zhuang, X.; Zhang, Y. Decoding Molecular and Cellular Heterogeneity of Mouse Nucleus Accumbens. Nat. Neurosci. 2021, 24 (12), 1757-1771.
7. Tepper, J. M.; Koós, T.; Ibanez-Sandoval, O.; Tecuapetla, F.; Faust, T. W.; Assous, M. Heterogeneity and Diversity of Striatal GABAergic Interneurons: Update 2018. Front. Neuroanat. 2018, 12, 91.
8. Avey, D.; Sankararaman, S.; Yim, A. K. Y.; Barve, R.; Milbrandt, J.; Mitra, R. D. Single-Cell RNA-Seq Uncovers a Robust Transcriptional Response to Morphine by Glia. Cell Rep. 2018, 24 (13), 3619-3629.e4.
9. Savell, K. E.; Tuscher, J. J.; Zipperly, M. E.; Duke, C. G.; Phillips, R. A., 3rd; Bauman, A. J.; Thukral, S.; Sultan, F. A.; Goska, N. A.; Ianov, L.; Day, J. J. A Dopamine-Induced Gene Expression Signature Regulates Neuronal Function and Cocaine Response. Sci. Adv. 2020, 6 (26), eaba4221.

### REFERENCES FOR EXAMPLE 3

1. Wang, X. et al., Three-dimensional intact-tissue sequencing of single-cell transcriptional states. Science 361, (2018).
2. Zeng, H. et al., Spatially Resolved Single-cell Translatomics at Molecular Resolution. bioRxiv 2022.09.27.509605 (2022) doi: 10.1101/2022.09.27.509605.
3. Lein, E., Borm, L. E. & Linnarsson, S. The promise of spatial transcriptomics for neuroscience in the era of molecular cell typing. Science 358, 64-69 (2017).
4. Fang, R. et al., Three-dimensional single-cell transcriptome imaging of thick tissues. Elife 12, (2023).
5. Wang, Y. et al., EASI-FISH for thick tissue defines lateral hypothalamus spatio-molecular organization. Cell 184, 6361-6377.e24 (2021).
6. Gandin, V. et al., Deep-tissue spatial omics: Imaging whole-embryo transcriptomics and subcellular structures at high spatial resolution. bioRxiv (2024) doi: 10.1101/2024.05.17.594641.
7. Yoshimura, Y. & Fujimoto, K. Ultrafast reversible photo-cross-linking reaction: toward in situ DNA manipulation. Org. Lett. 10, 3227-3230 (2008).
8. Yoshimura, Y., Ohtake, T., Okada, H. & Fujimoto, K. A new approach for reversible RNA photocrosslinking reaction: application to sequence-specific RNA selection. Chembiochem 10, 1473-1476 (2009).
9. Fujimoto, K., Konishi-Hiratsuka, K., Sakamoto, T. & Yoshimura, Y. Site-specific cytosine to uracil transition by using reversible DNA photo-crosslinking. Chembiochem 11, 1661-1664 (2010).
10. Zeisel, A. et al., Molecular Architecture of the Mouse Nervous System. Cell 174, 999-1014.e22 (2018).
11. Saunders, A. et al., Molecular Diversity and Specializations among the Cells of the Adult Mouse Brain. Cell 174, 1015-1030.e16 (2018).
12. Tasic, B. et al., Shared and distinct transcriptomic cell types across neocortical areas. Nature 563, 72-78 (2018).
13. Shi, H. et al., Publisher Correction: Spatial atlas of the mouse central nervous system at molecular resolution. Nature 625, E6 (2024).
14. He, Y. et al., Towards a universal spatial molecular atlas of the mouse brain. bioRxiv (2024) doi: 10.1101/2024.05.27.594872.
15. Dong, H. W. & The Allen Institute for Brain Science. The Allen Reference Atlas, (Book+CD-ROM): A Digital Color Brain Atlas of the C57BL/6J Male Mouse. (Wiley, 2008).
16. Website. Allen Mouse Brain Atlas https://mouse.brain-map.org/(2004).
17. Korsunsky, I. et al., Fast, sensitive and accurate integration of single-cell data with Harmony. Nat. Methods 16, 1289-1296 (2019).
18. Xu, C. S. et al., Enhanced FIB-SEM systems for large-volume 3D imaging. Elife 6, (2017).
19. Scheffer, L. K. et al., A connectome and analysis of the adult central brain. Elife 9, (2020).
20. Sakaguchi, R., Leiwe, M. N. & Imai, T. Bright multicolor labeling of neuronal circuits with fluorescent proteins and chemical tags. Elife 7, (2018).
21. Chan, K. Y. et al., Engineered AAVs for efficient noninvasive gene delivery to the central and peripheral nervous systems. Nat. Neurosci. 20, 1172-1179 (2017).
22. Guzman, A. K., Schmults, C. D. & Ruiz, E. S. Squamous Cell Carcinoma: An Update in Staging, Management, and Postoperative Surveillance Strategies. Dermatol. Clin. 41, 1-11 (2023).
23. Joost, S. et al., The Molecular Anatomy of Mouse Skin during Hair Growth and Rest. Cell Stem Cell 26, 441-457.e7 (2020).
24. Ji, A. L. et al., Multimodal Analysis of Composition and Spatial Architecture in Human Squamous Cell Carcinoma. Cell 182, 1661-1662 (2020).
25. Jerby-Arnon, L. et al., A Cancer Cell Program Promotes T Cell Exclusion and Resistance to Checkpoint Blockade. Cell 175, 984-997.e24 (2018).
26. Tirosh, I. et al., Dissecting the multicellular ecosystem of metastatic melanoma by single-cell RNA-seq. Science 352, 189-196 (2016).
27. Borden, E. S. et al., Neoantigen Fitness Model Predicts Lower Immune Recognition of Cutaneous Squamous Cell Carcinomas Than Actinic Keratoses. Front. Immunol. 10, 2799 (2019).
28. Levine, D. E., Karia, P. S. & Schmults, C. D. Outcomes of Patients With Multiple Cutaneous Squamous Cell Carcinomas: A 10-Year Single-Institution Cohort Study. JAMA Dermatol. 151, 1220-1225 (2015).
29. Gonzalez, J. L. et al., Multiple Cutaneous Squamous Cell Carcinoma in Immunosuppressed vs Immunocompetent Patients. JAMA Dermatol. 155, 625-627 (2019).
30. Shi, H. et al., Spatial atlas of the mouse central nervous system at molecular resolution. Nature 622, 552-561 (2023).
31. He, Y. et al., ClusterMap for multi-scale clustering analysis of spatial gene expression. Nat. Commun. 12, 5909 (2021).
32. Fujita, H. et al., Langerhans cells from human cutaneous squamous cell carcinoma induce strong type 1 immunity. J. Invest. Dermatol. 132, 1645-1655 (2012).
33. Klechevsky, E. et al., Functional specializations of human epidermal Langerhans cells and CD14+ dermal dendritic cells. Immunity 29, 497-510 (2008).

### INCORPORATION BY REFERENCE

The present application refers to various issued patent, published patent applications, scientific journal articles, and other publications, all of which are incorporated herein by reference. The details of one or more embodiments of the invention are set forth herein. Other features, objects, and advantages of the invention will be apparent from the Detailed Description, the Figures, the Examples, and the Claims.

### EQUIVALENTS AND SCOPE

In the articles such as “a,” “an,” and “the” may mean one or more than one unless indicated to the contrary or otherwise evident from the context. Embodiments or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process.

Furthermore, the disclosure encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claims that is dependent on the same base claim. Where elements are presented as lists, e.g., in Markush group format, each subgroup of the elements is also disclosed, and any element(s) can be removed from the group. It should it be understood that, in general, where the invention, or aspects of the invention, is/are referred to as comprising particular elements and/or features, certain embodiments of the disclosure or aspects of the disclosure consist, or consist essentially of, such elements and/or features. For purposes of simplicity, those embodiments have not been specifically set forth in haec verba herein. It is also noted that the terms “comprising” and “containing” are intended to be open and permits the inclusion of additional elements or steps. Where ranges are given, endpoints are included. Furthermore, unless otherwise indicated or otherwise evident from the context and understanding of one of ordinary skill in the art, values that are expressed as ranges can assume any specific value or subrange within the stated ranges in different embodiments of the invention, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the instant specification, the specification shall control. In addition, any particular embodiment of the present invention that falls within the prior art may be explicitly excluded from any one or more of the embodiments. Because such embodiments are deemed to be known to one of ordinary skill in the art, they may be excluded even if the exclusion is not set forth explicitly herein. Any particular embodiment of the invention can be excluded from any embodiment, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, but rather is as set forth in the appended embodiments. Those of ordinary skill in the art will appreciate that various changes and modifications to this description may be made without departing from the spirit or scope of the present invention, as defined in the following claims.

What is claimed is:

1. A method comprising:
a) contacting the tissue sample with 1) one or more sets of oligonucleotide probes, wherein each set of oligonucleotide probes comprises a first probe and a second probe, and 2) a third oligonucleotide probe, wherein
i) the first probe comprises a portion that is complementary to at least a portion of the third oligonucleotide probe, a portion that is complementary to an RNA of interest, and a portion that is complementary to a portion of the second probe;
ii) the second probe comprises a portion that is complementary to a portion of the first probe, a portion that is complementary to the RNA of interest, and an oligonucleotide barcode sequence; and
iii) the third probe comprises a portion that is complementary to the first probe and at least one crosslinking moiety;
b) co-polymerizing the one or more sets of probes, the third probe, and a polymeric matrix to form an oligonucleotide-polymeric matrix hybrid, wherein the first probe is hybridized to the RNA of interest;
c) removing unbound biomolecules from the oligonucleotide-polymeric matrix hybrid;
d) ligating the 5′ end and the 3′ end of the second probe together to produce a circular oligonucleotide;
e) performing rolling circle amplification to amplify the circular oligonucleotide using the first probe as a primer, thereby producing one or more concatenated amplicons;
f) embedding the one or more concatenated amplicons in the polymeric matrix; and
g) sequencing the concatenated amplicons, or a portion thereof, embedded in the polymeric matrix to determine the identity and location of each RNA of interest in the tissue sample.

2. The method of claim 1, wherein the tissue sample comprises tissue that is at least 20, at least 30, at least 40, at least 50, at least 100, at least 150, at least 200, at least 250, at least 300, or at least 350 μm thick.

3. The method of claim 1, wherein the first probe comprises the structure 5′-[portion complementary to portion of third probe]-[portion complementary to RNA of interest]-[portion complementary to portion of second probe]-3′.

4. The method of claim 1, wherein the second probe comprises the structure 5′-[portion complementary to portion of first probe]-[portion complementary to RNA of interest]-[oligonucleotide barcode sequence]-[portion complementary to portion of first probe]-3′.

5. The method of claim 1, wherein the third probe comprises the structure 5′-[first crosslinking moiety]-[portion complementary to first probe]-3′

6. The method of claim 1, wherein the portion of the third probe that is complementary to the first probe comprises a nucleoside analog comprising a second crosslinking moiety

7. The method of claim 6, wherein the second crosslinking moiety of the third probe is covalently crosslinked to the first probe.

8. The method of claim 1, wherein the third probe comprises an inverted nucleotide at its 3′ end.

9. The method of claim 1, wherein the set of probes comprises a fourth probe comprising a portion complementary to the third probe, a portion complementary to at least a portion of a ribosomal RNA (rRNA), and a portion complementary to the second probe.

10. The method of claim 9, wherein the portion of the fourth probe complementary to at least a portion of an rRNA is complementary to at least a portion of an 18S rRNA.

11. The method of claim 9, wherein the fourth probe comprises the structure 5′-[portion complementary to third probe]-[portion complementary to rRNA]-[portion complementary to portion of second probe]-3′.

12. The method of claim 9, wherein the second probe comprises the structure 5′-[portion complementary to portion of fourth probe]-[portion complementary to portion of first probe]-[portion complementary to RNA of interest]-[oligonucleotide barcode sequence]-[portion complementary to portion of fourth probe]-3′.

13. The method of claim 9, wherein the portion of the fourth probe that is complementary to the third probe comprises the same sequence as the portion of the first probe that is complementary to the third probe.

14. The method of claim 1, wherein the polymeric matrix is a hydrogel.

15. A set of oligonucleotide probes comprising:
i) a first probe comprising a portion that is complementary to at least a portion of the third probe, a portion that is complementary to an RNA of interest, and a portion that is complementary to a portion of the second probe;
ii) a second probe comprising a portion that is complementary to a portion of the first probe, a portion that is complementary to the RNA of interest, and an oligonucleotide barcode sequence; and
iii) a third probe comprising a portion that is complementary to the first probe and at least one crosslinking moiety.

16. The set of probes of claim 15, further comprising:
iv) a fourth probe comprising a portion complementary to the third probe, a portion complementary to at least a portion of a ribosomal RNA (rRNA) within a ribosome, and a portion complementary to the second probe.

17. A kit comprising the set of probes of claim 15.

18. The kit of claim 17, further comprising one or more of: (a) one or more enzymes.

19. A system for profiling RNA expression and/or translation in a cell comprising:
a) a tissue sample; and
b) one or more sets of oligonucleotide probes, wherein each set of probes comprises a first probe and a second probe, and a third oligonucleotide probe, wherein:
i) the first probe comprises a portion that is complementary to at least a portion of the third oligonucleotide probe, a portion that is complementary to an RNA of interest, and a portion that is complementary to a portion of the second probe;
ii) the second probe comprises a portion that is complementary to a portion of the first probe, a portion that is complementary to the RNA of interest, and an oligonucleotide barcode sequence; and
iii) the third probe comprises a portion that is complementary to the first probe and at least one crosslinking moiety.

20. The system of claim 19, wherein each set of probes further comprises:
iv) a fourth probe comprising a portion complementary to the third probe, a portion complementary to at least a portion of a ribosomal RNA (rRNA) within a ribosome, and a portion complementary to the second probe.


SEQUENCE LISTING (34 sequences)

SEQ ID NO: 1 - DNA, 22 bp, synthetic construct
  source 1..22: mol_type = other DNA
  modified_base 1: mod_base = OTHER; note = Modified with an acrydite group
  misc_feature 4: note = attached to the 5prime end of the 3-cyanovinylcarbazole nucleoside
  misc_feature 5: note = attached to the 3prime end of the 3-cyanovinylcarbazole nucleoside
gctaatacgtcgtactggtagg

SEQ ID NO: 2 - DNA, 25 bp, synthetic construct
  source 1..25: mol_type = other DNA
cctaccagtacgacgtatttagcaa

SEQ ID NO: 3 - DNA, 23 bp, synthetic construct
  source 1..23: mol_type = other DNA
  modified_base 5: mod_base = OTHER; note = nucleoside analog comprising second crosslinking moiety
gctanatacgtcgtactggtagg

SEQ ID NO: 4 - DNA, 22 bp, synthetic construct
  source 1..22: mol_type = other DNA
  modified_base 1: mod_base = OTHER; note = Modified with an acrydite group
  misc_feature 4: note = attached to the 5prime end of the 3-cyanovinylcarbazole nucleoside
  misc_feature 5: note = attached to the 3prime end of the 3-cyanovinylcarbazole nucleoside
gctaatacgtcgtactggtagg

SEQ ID NO: 5 - DNA, 59 bp, synthetic construct
  source 1..59: mol_type = other DNA
cctaccagtacgacgtatttagcaaggcttcccataggtgtatatgcgtggattagaaa

SEQ ID NO: 6 - DNA, 57 bp, synthetic construct
  source 1..57: mol_type = other DNA
cctaccagtacgacgtatttagcaagccggaagaccgatatttgggtggattagaaa

SEQ ID NO: 7 - DNA, 56 bp, synthetic construct
  source 1..56: mol_type = other DNA
cctaccagtacgacgtatttagcaacccgggtttgatgtaagccgtggattggaaa

SEQ ID NO: 8 - DNA, 59 bp, synthetic construct
  source 1..59: mol_type = other DNA
cctaccagtacgacgtatttagcaaggtcatctcacccatggatagggtggattggaaa

SEQ ID NO: 9 - DNA, 60 bp, synthetic construct
  source 1..60: mol_type = other DNA
cctaccagtacgacgtatttagcaacctggttttcttgtggcatgtgagtggattcgaaa

SEQ ID NO: 10 - DNA, 56 bp, synthetic construct
  source 1..56: mol_type = other DNA
cctaccagtacgacgtatttagcaacctgcagaaattggctcacgtggattcgaaa

SEQ ID NO: 11 - DNA, 57 bp, synthetic construct
  source 1..57: mol_type = other DNA
cctaccagtacgacgtatttagcaaatcagctctctcgctttagagtggatttgaaa

SEQ ID NO: 12 - DNA, 56 bp, synthetic construct
  source 1..56: mol_type = other DNA
cctaccagtacgacgtatttagcaatcccatttggccttaccctgtggatttgaaa

SEQ ID NO: 13 - DNA, 56 bp, synthetic construct
  source 1..56: mol_type = other DNA
atccaccacacggacacattcatctctaattacttttccatacactaaagtttcta

SEQ ID NO: 14 - DNA, 56 bp, synthetic construct
  source 1..56: mol_type = other DNA
atccacggaagaccaaatccaccttgtaattacttttccatacactaaagtttcta

SEQ ID NO: 15 - DNA, 55 bp, synthetic construct
  source 1..55: mol_type = other DNA
atccactgacatgtttcagaacgggcaattacaaaagcatacactaaagtttcca

SEQ ID NO: 16 - DNA, 56 bp, synthetic construct
  source 1..56: mol_type = other DNA
atccacagaagtcgagagccgaaggataattacaaaagcatacactaaagtttcca

SEQ ID NO: 17 - DNA, 56 bp, synthetic construct
  source 1..56: mol_type = other DNA
atccacccactgacgcatttctcccagaattacaaaaccatacactaaagtttcga

SEQ ID NO: 18 - DNA, 55 bp, synthetic construct
  source 1..55: mol_type = other DNA
atccaccactcttgggtcagctgattaattacaaaaccatacactaaagtttcga

SEQ ID NO: 19 - DNA, 55 bp, synthetic construct
  source 1..55: mol_type = other DNA
atccactataggcacacatggcatctaattacaaaatcatacactaaagtttcaa

SEQ ID NO: 20 - DNA, 57 bp, synthetic construct
  source 1..57: mol_type = other DNA
atccacagatctaacattgctgggcatgaattacaaaatcatacactaaagtttcaa

SEQ ID NO: 21 - DNA, 112 bp, synthetic construct
  source 1..112: mol_type = other DNA
cctaccagtacgacgtatttagcaaacaaaatagaaccgcggtcctattcaaaaaaaaaaaaaaaaaaaaaaaaaaaaaaaaaaaaaaaaaaaaaaaaaatatctttagtgt

SEQ ID NO: 22 - DNA, 112 bp, synthetic construct
  source 1..112: mol_type = other DNA
cctaccagtacgacgtatttagcaacatcgtttatggtcggaactacgacaaaaaaaaaaaaaaaaaaaaaaaaaaaaaaaaaaaaaaaaaaaaaaaaaatatctttagtgt

SEQ ID NO: 23 - DNA, 111 bp, synthetic construct
  source 1..111: mol_type = other DNA
cctaccagtacgacgtatttagcaaaggtttcccgtgttgagtcaaattaaaaaaaaaaaaaaaaaaaaaaaaaaaaaaaaaaaaaaaaaaaaaaaaaatatctttagtgt

SEQ ID NO: 24 - DNA, 112 bp, synthetic construct
  source 1..112: mol_type = other DNA
cctaccagtacgacgtatttagcaatgttattgctcaatctcgggtggctaaaaaaaaaaaaaaaaaaaaaaaaaaaaaaaaaaaaaaaaaaaaaaaaaatatctttagtgt

SEQ ID NO: 25 - DNA, 112 bp, synthetic construct
  source 1..112: mol_type = other DNA
cctaccagtacgacgtatttagcaaagatagtcaagttcgaccgtcttctaaaaaaaaaaaaaaaaaaaaaaaaaaaaaaaaaaaaaaaaaaaaaaaaaatatctttagtgt

SEQ ID NO: 26 - DNA, 60 bp, synthetic construct
  source 1..60: mol_type = other DNA
aagataaatttctaatcctacacacggacacattcatctctattacttttccatacacta

SEQ ID NO: 27 - DNA, 60 bp, synthetic construct
  source 1..60: mol_type = other DNA
aagataaatttctaatcctaggaagaccaaatccaccttgtattacttttccatacacta

SEQ ID NO: 28 - DNA, 59 bp, synthetic construct
  source 1..59: mol_type = other DNA
aagataaatttccaatcctatgacatgtttcagaacgggcattacaaaagcatacacta

SEQ ID NO: 29 - DNA, 60 bp, synthetic construct
  source 1..60: mol_type = other DNA
aagataaatttccaatcctaagaagtcgagagccgaaggatattacaaaagcatacacta

SEQ ID NO: 30 - DNA, 56 bp, synthetic construct; identical to SEQ ID NO: 17
  source 1..56: mol_type = other DNA

SEQ ID NO: 31 - DNA, 55 bp, synthetic construct; identical to SEQ ID NO: 18
  source 1..55: mol_type = other DNA

SEQ ID NO: 32 - DNA, 59 bp, synthetic construct
  source 1..59: mol_type = other DNA
aagataaatttcaaatcctatataggcacacatggcatctattacaaaatcatacacta

SEQ ID NO: 33 - DNA, 61 bp, synthetic construct
  source 1..61: mol_type = other DNA
aagataaatttcaaatcctaagatctaacattgctgggcatgattacaaaatcatacacta

SEQ ID NO: 34 - DNA, 23 bp, synthetic construct
  source 1..23: mol_type = other DNA
  modified_base 5: mod_base = OTHER; note = nucleoside analog comprising second crosslinking moiety
gctanatacgtcgtactggtagg